(19) United States
(12) Patent Application Publication
(10) Pub. No.: US 2024/0257899 A1
(43) Pub. Date: Aug. 1, 2024

(54) METHOD, DEVICE, AND PROGRAM FOR SEARCHING FOR NEW DIAGNOSTIC BIOMARKER AND/OR THERAPEUTIC TARGET

(71) Applicant: KARYDO THERAPEUTIX, INC., Tokyo (JP)

(72) Inventor: Narutoku Sato, Kyoto (JP)

(73) Assignee: KARYDO THERAPEUTIX, INC., Tokyo (JP)

(21) Appl. No.: 18/561,822

(22) PCT Filed: May 18, 2022
(86) PCT No.: PCT/JP2022/020744

§ 371 Date: Nov. 17, 2023

(30) Foreign Application Priority Data
May 18, 2021 (JP) 2021-084114

(51) Int. Cl. G16B 5/00; G16B 15/30; G16B 40/00; G16H 50/20

(52) U.S. Cl. CPC G16B 5/00; G16B 15/30; G16B 40/00; G16H 50/20

(57) ABSTRACT

A method of searching for novel diagnostic biomarkers and/or therapeutic targets, comprising performing a topic analysis using a topic model on an omics database derived from two or more biofeatures linked to information indicating a disease name, to estimate missing biofeatures for a particular disease, wherein the estimated missing biofeatures suggest the novel diagnostic biomarkers and/or therapeutic targets, is provided.

### TECHNICAL FIELD

Disclosed herein are a method, device and program of searching for novel diagnostic biomarkers and/or therapeutic targets.

### BACKGROUND ART

Computational methods for predicting potential biomarkers and/or therapeutic targets for human diseases have revolutionized medicine and drug development. One of such approaches is graph theory-based methods. This builds a network of diseases on the basis of a single type of disease-associated information, such as genetic influences on diseases, to allow for detecting genetic biomarkers according to the genetic relevance of the diseases. The same framework can also be used to predict novel therapeutic targets of diseases on the basis of other information, such as drug targets. Another approach is based on natural language processing models (Non-Patent Literatures 1 and 2). This method builds a model for searching for “likely” biomarkers and therapeutic targets by using descriptions of biological processes in numerous publications and other documents.

### CITATION LIST

### Non Patent Literature

Non Patent Literature 1: Gyori, B. M., et al. (2017). Molecular systems biology 13, 954.

Non Patent Literature 2: Haggerty, R. A., and Purvis, J. E. (2017). Molecular systems biology 13, 958.

### SUMMARY OF INVENTION

### Technical Problem

The above approaches are common and, in some cases, effective in finding biomarkers and therapeutic targets. However, diseases are influenced by many factors, including genes, proteins, cell-cell interactions, lifestyle, environment, microbiota in the human body, and the like. Hence, it would be more useful to have a method that can identify latent disease-disease associations based on a plurality of disease-omics data. Such an approach may facilitate the discovery of biomarkers and/or therapeutic targets in the case where a plurality of disease-associated factors caused the disease. For example, a close disease-disease relationship based on genetics could be used to discover lifestyle biomarkers for the same diseases. A close disease-microbiota relationship may be translated into related drug targets.

An object of the present invention is to provide a framework for searching for biomarkers and/or therapeutic targets based on a plurality of types of disease-omics data.

### Solution to Problem

The present invention may include following embodiments.

### Item 1.

A method of searching for novel diagnostic biomarkers and/or therapeutic targets, comprising
performing a topic analysis using a topic model on an omics database derived from two or more biofeatures linked to information indicating a disease name to estimate missing biofeatures for a particular disease, wherein
the estimated missing biofeatures suggest the novel diagnostic biomarkers and/or therapeutic targets.

### Item 2.

The method according to item 1, wherein the two or more biofeatures are selected from single nucleotide polymorphisms, microbiota, drug targets, transcriptome, proteome and disease biomarkers.

### Item 3.

The method according to item 1, wherein the topic model is based on Latent Dirichlet Allocation model.

### Item 4.

The method according to item 1, further comprising verifying the reliability of the extracted missing biofeatures.

### Item 5.

A device of searching for novel diagnostic biomarkers and/or therapeutic targets provided with a control unit, wherein
the control unit performs a topic analysis using a topic model on an omics database derived from two or more biofeatures linked to information indicating a disease name to estimate missing biofeatures for a particular disease, wherein
the estimated missing biofeatures suggest the novel diagnostic biomarkers and/or therapeutic targets.

### Item 6.

A program of searching for novel diagnostic biomarkers and/or therapeutic targets that, when executed by a computer, causes the computer to execute a step of performing a topic analysis using a topic model on an omics database derived from two or more biofeatures linked to information indicating a disease name to estimate missing biofeatures for a particular disease, wherein
the estimated missing biofeatures suggest the novel diagnostic biomarkers and/or therapeutic targets.

### Item 7.

A method of using relationship between each disease and combination of tissue and gene corresponding to each disease listed in Listing 3 in the present description as a candidate for disease biomarkers and/or therapeutic targets.

### Advantageous Effects of Invention

A framework for searching for novel diagnostic biomarkers and/or therapeutic targets is provided.

### BRIEF DESCRIPTION OF DRAWINGS

FIG. 1 shows a configuration of a main database.

FIG. 2 shows a concept of a topic model.

FIG. 3 shows a hardware configuration of search device 10.

FIG. 4 shows a flow of processing in search program 1042.

FIG. 5 shows validation results of leave one-disease features out cross-validation.

FIG. 6-1 shows candidates for novel diagnostic biomarkers and/or therapeutic targets.

FIG. 6-2 shows candidates for the novel diagnostic biomarkers and/or therapeutic targets.

FIG. 6-3 shows candidates for the novel diagnostic biomarkers and/or therapeutic targets.

FIG. 6-4 shows candidates for the novel diagnostic biomarkers and/or therapeutic targets.

FIG. 6-5 shows candidates for the novel diagnostic biomarkers and/or therapeutic targets.

FIG. 6-6 shows candidates for the novel diagnostic biomarkers and/or therapeutic targets.

FIG. 6-7 shows candidates for the novel diagnostic biomarkers and/or therapeutic targets.

FIG. 6-8 shows candidates for the novel diagnostic biomarkers and/or therapeutic targets.

FIG. 6-9 shows candidates for the novel diagnostic biomarkers and/or therapeutic targets.

### DESCRIPTION OF EMBODIMENTS

1. Search of Novel Diagnostic Biomarkers and/or Therapeutic Targets

One embodiment of the present invention relates to search of novel diagnostic biomarkers and/or therapeutic targets.

### 1-1. Main Database

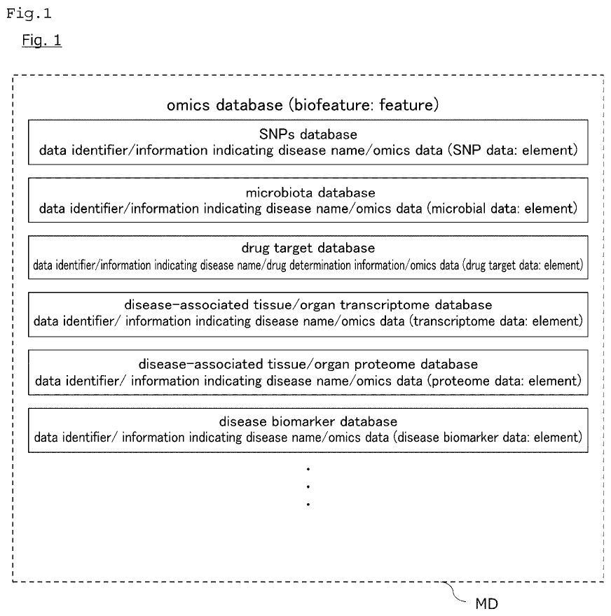

In this embodiment, main database MD is first prepared. The main database MD stores omics databases derived from different biofeatures, as shown in FIG. 1. The biofeatures are intended to be biological indicators associated with living organisms. An omics data is intended to be an individual omics data belonging to each biofeature. That is, the term ‘biofeature’ encompasses and describes certain biological indicators as features, as well as may include the individual omics data (the elements that make up the feature) that make up the feature. The language in which the database is structured is not limited, but is preferably English. However, the omics data corresponding to information indicating a disease name may be singular or plural. Hereafter, this definition is invoked herein.

The biofeature, for example, may be single nucleotide polymorphisms (SNPs), microbiota, drug target, transcriptome, proteome, disease biomarker transcriptome, proteome, disease biomarkers, or the like. In FIG. 1, illustratively, SNPs database, Microbiota database, drug target database, disease-associated tissue/organ transcriptome database, disease-associated tissue/organ proteome database, and disease biomarker database are shown.

Here, the individual omics data contained in each database is linked to the information indicating the disease names. In addition, the information indicating the disease name is assigned a data identifier to identify the information indicating the individual disease.

The information indicating the disease name includes information that can at least identify the disease name. The information that can identify the disease name may include the disease name itself, an abbreviation, the disease name and its type or stage, and so on. The term “disease” used in the present description may also include symptoms in addition to those defined as a disease.

For example, the SNPs database stores one data unit or a plurality of data units, wherein the one data unit is a series of data linking the information indicating the disease name to which the data identifier is assigned and the omics data, i.e., SNP data or SNPs data, linked to the information indicating the disease name, and the plurality of data units correspond to information indicating different disease names. The SNP data is, for example, accession number (SNP_GENE_IDS*: Entrez Gene ID), rs number, or chromosome number associated with the rs number. Disease-associated SNPs database can be obtained, for example, from the GWAS Catalog (https://www.ebi.ac.uk/gwas/docs/file-downloads) under “All associations” (v1.0.2).

For example, the microbiota database stores one data unit or a plurality of data units, wherein the one data unit is a series of data linking the information indicating the disease name to which the data identifier is assigned, and the omics data, i.e., microbial data, linked to the information indicating the disease name, and the plurality of data units correspond to information indicating different disease names. The microbial data is, for example, the name of a microorganism and the like. Disease-associated microbiota database can be obtained, for example, from Disbiome (version on 11th of November 2020, https://disbiome.ugent.be/home) (Ref: Janssens et al., 2018, Disbiome database: linking the microbiome to disease. BMC Microbiol 18,50).

For example, the drug target database stores one or a plurality of data units, wherein the one data unit is a series of data linked to the information indicating the disease name to which the data identifier is assigned, drug determination information linked to the information indicating the disease name, and the omics data, i.e., drug target data, linked to the drug determination information, and the plurality of data units correspond to information indicating different disease names. The drug determination information is, for example, a drug name, a compound name, and the like. The drug target data is, for example, the name of a target molecule, and the like (protein name, cytokine name, chemokine name, and the like). In the drug target database, by first, “therapeutic-targets (i.e., diseases) information for drugs from SIDER (version 4.1) (http://sideeffects.embl.de)”, for example, is downloaded, then “drug-targets from DrugBank (https://go.drugbank.com/)” is searched to associate the drug determination information linked to the information indicating the disease name with the drug target data linked to the drug determination information. This can be done, for example, using web-scraping on Python (version 3.8.1).

For example, a disease-associated tissue/organ transcriptome database stores one data unit or a plurality of data units, wherein the one data unit is a series of data linked to the information indicating the disease name to which the data identifier is assigned, and the omics data, i.e., transcriptome data, linked to the information indicating the disease name, and the plurality of data units correspond to information indicating different disease names. The transcriptome data is the name of an organ and/or tissue, and the transcriptome behavior data for each organ and/or tissue, i.e., various gene names and expression levels thereof, and the like.

The tissue/organ transcriptome database can be generated by the following procedure. For example, NCBI GEO DataSets (https://www.ncbi.nlm.nih.gov/gds) can be collected using a keyword. The keyword can be, for example, “‘RNA-seq” [All Fields] AND “Homo sapiens” [organism] NOT “scRNA” [All Fields] NOT “Single cell” [All Fields] NOT “culture” [All Fields] NOT “cultured” [All Fields] NOT “cell line” [All Fields] NOT “treatment” [All Fields] NOT “single-nucleus” [All Fields] NOT “microRNA” [All Fields] NOT “iPSC” [All Fields] NOT “organoids” [All Fields] AND “expression profiling by high throughput sequencing” [DataSet Type] AND “SIDER's indication” [All Fields]’. In this case, only the input term “SIDER's indication” may be automatically converted into the above keywords. The “SIDER's indication” list is available for download from SIDER 4.1 (http://sideeffects.embl.de). After making the initial selection of GEO datasets described above automatically, GEO datasets containing both Human disease-associated bulk tissue/organ transcriptome data and control or healthy data in the same project are manually selected, and consequently, for example, 50 GEO datasets useful for this analysis are obtained. Examples of selected datasets are given in <List 1: GEO datasets> at the end of the present description.

Raw data for each selected GEO dataset can then also be generated. For example, the raw data for 17 GEO datasets (GSE107638, GSE112057, GSE112523, GSE114007, GSE117970, GSE121212, GSE124439, GSE131031, GSE131525, GSE133624, GSE134056, GSE138082, GSE138614, GSE146889, GSE42546, GSE80336, GSE80655) can be obtained from NCBI GEO DataSets websites. The raw data for the remaining 33 GEO datasets can be generated from FASTQ data. To briefly describe the generation of raw data from FASTQ data, at the first, the FASTQ data is downloaded from European Nucleotide Archive (ENA; https://www.ebi.ac.uk/ena/browser/home) via FTP downloading or the fast file-downloading software, Aspera Connect (https://www.ibm.com/aspera/connect/; version 3.11.1.58). Next, in order to generate BAM data, for example, the downloaded FASTQ data is mapped onto human genome (UCSC hg 38) with STAR (version 2.7.8a). The generated BAM data is converted into raw count data by, for example, the human gene GTF file (UCSC hg 38). Specifically, this data conversion was performed by using the software of Subread (version 2.0.1), where the ‘featureCounts’ command is used with specifying the option ‘-Q10’ and, only in the case that the data is paired-end, additionally with specifying the options, ‘-p’ and ‘-B’. The processing of STAR and Subread can be performed on Linux of CentOS6 or CentOS7. Since some raw count data is stored as split FASTQ data, a plurality of raw count data may be combined into one by GSM ID, if necessary.

Next, differentially expressing genes (DEGs) are extracted. The extraction of the DEGs can be performed with DESeq2 (version 1.30.1) (Ref: Love et al., 2014, Moderated estimation of fold change and dispersion for RNA-seq data with DESeq2. Genome biology 15, 550) on free software R (version 4.0.3). DESeq2 analysis is conducted for the respective GSE project. First, transcriptome data is classified into a control group obtained from healthy tissue or normal tissue parts and a disease group derived from lesion site of each tissue. This classification can be performed on sample information described in NCBI GEO datasets. Then, DESeq2 analysis is performed for each disease in each GSE project. To select DEGs, genes are typically filtered by an adjusted p-value of less than 0.0001. However, if filtering by the adjusted p-value of less than 0.0001 does not yield any DEGs, the adjusted p-value threshold may be increased to 0.0001, 0.001, 0.01, or 0.05 until at least one DEG is obtained. Furthermore, tissue/organ names are manually unified into a rough class.

The tissue/organ proteome database stores one data unit or a plurality of data units, wherein the one data unit is a series of data linking the information indicating the disease name to which the data identifier is assigned, and the omics data, i.e., proteome data, linked to the information indicating the disease name, and the plurality of data units correspond to information indicating different disease names. The proteome data is, for example, the name of an organ and/or tissue, and the proteome behavior data for each organ and/or tissue, and the like.

The disease biomarker database stores one data unit or a plurality of data units, wherein the one data unit is a series of data linking the information indicating the disease name to which the data identifier is assigned, and the omics data, i.e., disease biomarker data, linked to the information indicating the disease name, and the plurality of data units correspond to information indicating different disease names. The disease biomarker data is, for example, data linking a specific disease to a biomarker name and its behavior, and to an organ or tissue name if necessary.

Here, the information indicating the name of the disease linked to each omics data and the data identifier assigned to it are unified across all databases. For example, the data identifier can be a Unified Medical Language System (UMLS) ID or the actual disease name. Specifically,

Unified Medical Language System (UMLS) IDs are added to disease names to combine the disease names acquired from a group of a plurality of independent data sources. The UMLS ID annotation can be performed, for example, by using ‘UMLS_AUI.extract_terminology (“ICD10”)’ function derived from python library PyMedTermino (version 0.3.3) (Ref: Lamy et al., 2015, Stud Health Technol Inform 210, 924-928). Prior to executing this function, replace the string “s” in the disease name is replaced with a blank space. Following assigning the UMLS ID annotations, if the UMLS IDs have the same disease name, the UMLS IDs are combined with the string ‘|’. For disease names for which this UMLS ID annotation failed, the actual disease name in the dataset is used and only those with the exact matching name are combined.

### 1-2. Topic Model

A topic model used in this embodiment is not limited as long as the information indicating the disease name and the omics data can be analyzed by linguistic analysis.

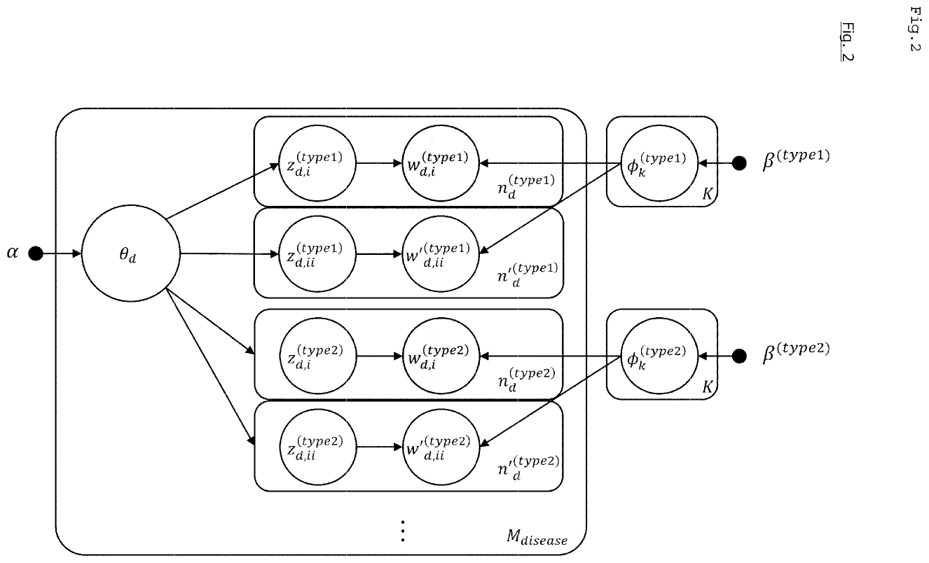

Here, an example of a model in which the Latent Dirichlet Allocation (LDA) model (Ref: Blei et al., 2003, Journal of Machine Learning Research 3, 993-1022), that is a conventional topic model for bag-of-words data, is extended to be able to classify diseases according to, for example, omics data derived from different biofeatures, is presented. This model is referred to herein as an extended LDA model. FIG. 2 shows the concept of the extended LDA model. In FIG. 2, empty circles represent random variables, grey circles represent observed/input data and black dots represent hyper-parameters.

In the extended LDA model, w_{di}^{(type)} represents the i-th information of disease d acquired from (type) information. z_{di}^{(type)} represents the topic number of w_{di}^{(type)}. θ_{d} represents the topic probability of disease d. ϕₖ^{(type)} represents the word probability of topic k in the (type) information. The total number of topic K is a hand-tuned parameter.

Wherein, the “type” is SNPs, drug-targets, microbiota, transcriptome, or the like. For example, the K can be set to “3”. Examples of methods to determine the number of topics include Perplexity, community detection, and the like.

The generative model of proposed topic models was defined as following.

$$Mathematical formula 1$$

$$p ( {w}_{di}^{( type )} \vert {z}_{di}^{( type )} , {ϕ}^{( type )} ) = Multinomial ( {w}_{di}^{( type )} \vert {ϕ}^{( type )} ) = {\prod }_{v = 1}^{{V}^{( type )}} {\prod }_{k = 1}^{K} {ϕ}_{vk}^{{( type )}_{vk}^{δ ( {z}_{di}^{( type )} = k ) δ ( {w}_{di}^{( type )} = v )}}$$

$$p ( {z}_{di}^{( type )} \vert {θ}_{d} ) = Multinomial ( {z}_{di}^{( type )} \vert {θ}_{d} ) = {\prod }_{k = 1}^{K} {θ}_{dk}^{δ ( {z}_{di}^{( type )} = k )}$$

$$p ( {θ}_{d} \vert α ) = Dirchlet ( {θ}_{d} \vert α ) = \frac{Γ ( {\sum }_{k} {α}_{k} )}{{\prod }_{k}^{} Γ ( {α}_{k} )} {\prod }_{k = 1}^{K} {θ}_{dk}^{{α}_{k} - 1}$$

$$p ( {ϕ}_{k}^{( type )} \vert {β}^{( type )} ) = Dirchlet ( {ϕ}_{k}^{( type )} \vert {β}^{( type )} ) = \frac{Γ ( {\sum }_{v} {β}_{v}^{( type )} )}{{\prod }_{v}^{{V}^{( type )}} Γ ( {β}_{v}^{( type )} )} {\prod }_{v = 1}^{{V}^{( type )}} {ϕ}^{{( type )}_{kv}^{{β}_{v}^{( type )} - 1}}$$

Wherein, α∈ [symbol] ^{+K} and β^{(type)}∈ [symbol] ^{+V}^{^{(type)}} are the hyperparameters and set to a vector with elements of 0.1. In addition, v is an index word, V^{(type)} is all types of words in a (type) data score. Γ(·) is the Gamma function and δ(·) is the Kronecker delta function.

Based on this model, the variables z_{di}^{(type)}, θ_{d} and ϕₖ^{(type)} are estimated. Some w_{di}^{(type)}, which are some (type) data sources using Gibbs sampling method, are also estimated based on the above model.

Conditional distributions are calculated based on, for example, Gibbs sampling method technique as described below.

$$Mathematical formula 2$$

$$p ( {z}_{di}^{( type )} = k \vert {w}_{di}^{( type )} = v , {θ}_{d} , {ϕ}^{( type )} ) = \frac{{θ}_{dk} {ϕ}_{kv}^{( type )}}{{\sum }_{{k}^{′}} {θ}_{{dk}^{′}} {ϕ}_{{k}^{′} v}^{( type )}}$$

$$p ( {θ}_{d} \vert {z}^{( type )} , α ) = Dirichlet ( {θ}_{d} \vert {α}^{′} ) ∝ {\prod }_{k = 1}^{K} {θ}_{dk}^{{α}_{k} + {\sum }_{t} {\sum }_{i} δ ( {z}_{di}^{( type )} = k ) - 1}$$

$$p ( {ϕ}_{k}^{( type )} \vert {z}^{( type )} , {w}^{( type )} , {β}^{( type )} ) = Dirichlet ( {ϕ}_{k}^{( type )} \vert {β}^{{( type )}^{′}} ) ∝ {\prod }_{v} {ϕ}^{{( type )}_{kv}^{{β}_{k}^{( type )} + {\sum }_{d} {\sum }_{i} δ ( {z}_{di}^{( type )} = k ) δ ( {w}_{di}^{( type )} = v ) - 1}}$$

$$p ( {w}_{di}^{( type )} = v \vert {z}_{di}^{( type )} = k , {ϕ}^{( type )} ) = {ϕ}_{kv}^{( type )}$$

Here, from this conditional distribution, sampling is repeated for samples z_{di}^{(type)}, θ_{d}, and ϕₖ^{(type)}, and w_{di}^{(type)}, until they all converge. For example, the number of iterations is set to 5,000, which is determined manually. The initial value of θ_{d} and ϕₖ^{(type)} are set to uniform distribution. The initial value of w_{di}^{(type)} is set to a randomly sampled word from ϕₖ^{(type)}. The total number of w_{di}^{(type)} at disease d is determined by sampling from binomial distribution. The parameters of the binomial distribution can be estimated by maximum likelihood estimation method by using only observed data.

The estimated variables z_{di}^{(type)}, θ_{d} and ϕₖ^{(type)} can be predicted by averaging sampled variables.

### 1-3. Search Device

One embodiment of the present invention relates to a search device 10 for novel diagnostic biomarkers and/or therapeutic targets (hereinafter simply referred to as “search device 10”).

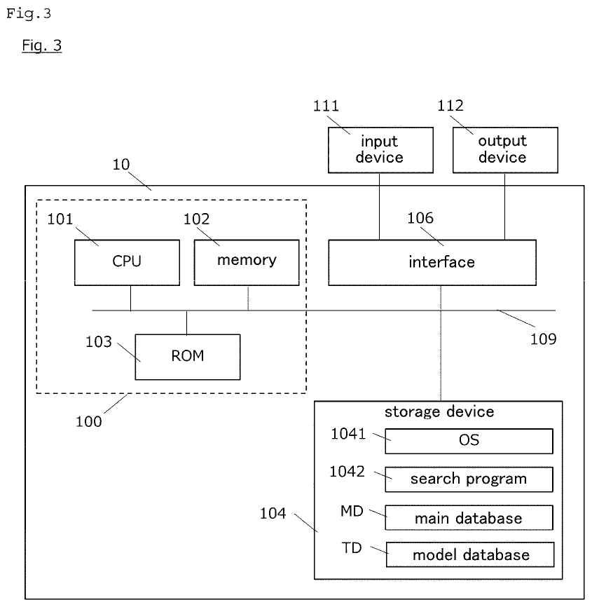

FIG. 3 shows a hardware configuration of the search device 10.

The search device 10 may be connected to an input device 111 and an output device 112.

In the search device 10, a processing unit (CPU) 101, memory 102, ROM (read only memory) 103, storage device 104, and interface 106 are connected to each other by a bus 109 so as to enable data communication. The processing unit 101, memory 102, and ROM 103 function as a control unit 100 of the search device 10.

The processing unit 101 is the CPU of the search device 10, also referred to as an arithmetic unit. The processing unit 101 may cooperate with a GPU or MPU. A computer functions as the search device 10 when the processing unit 101 executes a search program 1042 described below in cooperation with an operation system (OS) 1041 stored in the storage device 104 or ROM 103.

The ROM 103 stores the search program 1042 executed by the processing unit 101 and data used in it. The ROM 103 stores a boot program executed by the processing unit 101 when the search device 10 is started and programs and settings related to an operation of the hardware of the search device 10.

The storage device 104 non-volatilely stores the operation system (OS) 1041, the search program 1042 for novel diagnostic biomarkers and/or therapeutic targets (hereinafter simply referred to as “search program 1042”) described below, the main database MD described in the above 1-1. and a model database TD storing the topic models described in the above 1-2. The storage device 104 may also store analysis results.

The input device 111 is composed of a touch panel, keyboard, mouse, pen tablet, microphone, and the like, and provides text or voice input to the search device 10. The input device 111 may be connected from outside the search device 10 or may be integrated with the search device 10.

The output device 112 is composed of, for example, a display, printer, and the like.

The processing unit 101 may obtain an application software and various settings necessary to control the search device 10 via a network, instead of reading them from the ROM 103 or storage device 104. It is also possible for said application program to be stored in a storage device of a server computer on the network, and for the search device 10 to access this server computer and download the search program 1042, which is then stored in the ROM 103 or storage device 104.

An operating system providing a graphical user interface environment, for example, Windows® manufactured and sold by Microsoft Corporation in the United States, open source Linux, or the like, is installed in the ROM 103 or storage device 104. The search program runs on said operating system. Here, the search device 10 can be a personal computer or the like.

### 1-4. Processing in Search Program

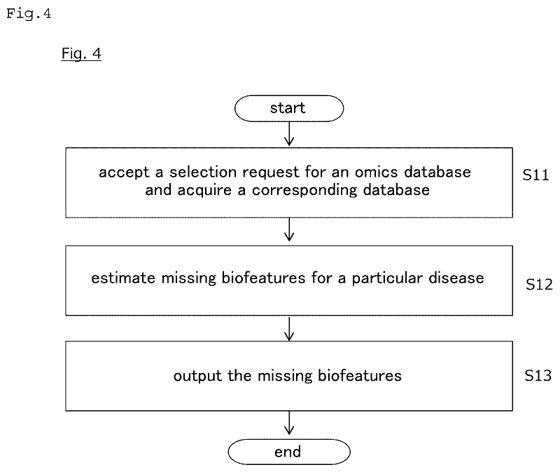

Referring to FIG. 4, the processing performed by the search program 1042 is described. The method of searching for novel diagnostic biomarkers and/or therapeutic targets is realized by the control unit 100 executing the search program 1042.

The control unit 100 accepts a request to start processing that is input by an operator from the input device 111, and starts processing.

In step S11, the control unit 100 accepts a selection request for the omics database input by the operator and acquires a corresponding omics database from the main database MD. At this time, the operator makes selection requests for at least two omics databases.

In step S12, the control unit 100 estimates missing biofeatures for the two or more omics databases acquired in the step S11 using the topic model stored in the model database TD. Here, “estimating” includes “extracting” biofeatures.

In step S13, the control unit 100 outputs the missing biofeatures together with information indicating a corresponding disease name, and terminates the processing.

Furthermore, the control unit 100 may perform a processing to verify the reliability of the extracted missing biofeatures. The verification processing can be performed, for example, by leave-one-disease features out cross-validation (which extracts all omics data for one disease and evaluates the proportion at which the missing omics data are extracted as missing biofeatures).

1-5. Recording Media with Search Programs Recorded Thereon One embodiment of the present invention relates to a program product, such as a media drive, that stores the search program 1042. That is, the search program 1042 can be stored in a media drive such as a hard disk, semiconductor memory element such as a flash memory, or an optical disk. The media drive can also be a computer such as a server device. The format of recording the program to the media drive is not limited as long as each device can read the program. It is preferred that the recording to the media drive is non-volatile.
2. Method of Using Relationship Between Each Disease and Combination of Tissue and Gene Corresponding to Each Disease Listed in Listing 3 as Candidate for Disease Biomarker and/or Therapeutic Target

In the Listing 3 at the end of the present description, the genes, tissues and diseases extracted in the above 1. are shown. These genes are candidates for novel diagnostic biomarkers and/or therapeutic targets. Here, the candidate is intended to be data in a state of crude refinement that has not yet been verified for actual effectiveness. A diverse and astronomical variety of omics data is obtained from living organisms, from which a vast number of experiments (screening) must be repeated to arrive at a specific diagnostic biomarker and/or therapeutic target. The candidate is intended to be a molecule that provides stepping-stone information to simplify such screening.

The Listing 3 lists the data in order of (Disease ID for figure, UMLS ID, Tissue_gene, count). The “Disease ID for figure” denotes the “Disease ID for figure” of the disease name “Disease” shown in Listing 2 at the end of the present description. The “UMLS ID” denotes the “UMLS ID” shown in Listing 1. The “Tissue_gene” denotes a combination of newly extracted tissue and genes. Thus, the “Tissue_gene” combinations in the Listing 3 are combinations of diagnostic biomarker and/or therapeutic target and tissue as new candidates in each disease.

### 3. Verification of Effectiveness

The LDA model is a topic model of bag-of-words data (Ref: Blei et al., 2003, Journal of Machine Learning Research 3, 993-1022). The conventional LDA model is a method that classifies sentences or documents as topics based on related words. Diseases are influenced by many different types of factors, including genes, microbiota, lifestyle, and the like. This is synonymous with describing diseases using terms such as genes, microbiota, lifestyle, and the like.

To address the multifactorial nature of human disease, the conventional LDA model was extended to classify diseases according to factors affecting a plurality of diseases. Herein, GWAS catalog (https://www.ebi.ac.uk/gwas/docs/file-downloads), SIDER (http://sideeffects.embl.de)/DrugBank (https://go.drugbank.com/), Disbiome (https://disbiome.ugent.be/home) (Janssens et al., 2018), or NCBI GEO DataSets (https://www.ncbi.nlm.nih.gov/gds) were used to classify diseases into three topics according to single nucleotide polymorphism (SNPs), drug targets (i.e., therapeutic targets of the disease), microbiota, and/or human disease-associated tissue/organ transcriptome data. The “number of topics” was determined by how many groups (topics) can be divided into to best represent each group (topic) specifically when the diseases were divided into groups (topics) according to the multiple biofeatures associated with each disease. Examples of methods for determining the number of topics include perplexity, community detection, and the like.

### 3-1. Performance Evaluation of Extended LDA Model

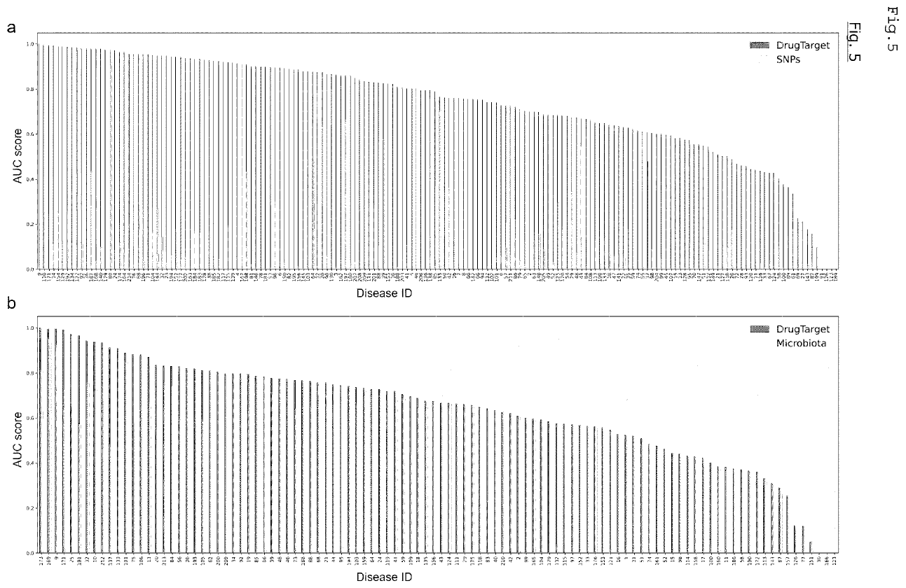

An extended LDA framework was used to predict disease biomarkers and/or therapeutic targets. For this purpose, performance in predicting the missing information for each disease was assessed by omitting certain types of disease-associated information and leaving the remaining information intact. Two models were evaluated by executing this “leave-one-disease features out cross-validation” scheme (FIG. 5, and Listing 2 at the end of the present description).

The first model is composed of the disease-associated biofeatures, i.e., SNPs and drug targets, and in this model, both SNPs and drug target biofeatures are tagged for 170 diseases with both biofeatures (i.e., the “type” in the description of the extended LDA model described above is “SNP and drug target”). The model includes 170 diseases with both SNPs and drug target information (FIG. 5, <Listing 2: Disease ID> at the end of the present description). All SNPs associated with one disease were excluded, all SNPs associated with the remaining 169 diseases were retained, and all drug target information was retained for all the 170 diseases. The generated model was then used to evaluate the performance of the model by predicting missing SNPs associated with the one disease mentioned above. This evaluation was repeated for all the 170 diseases.

In addition, all drug targets associated with one disease were excluded, all drug targets associated with the remaining 169 diseases were retained, and all SNPs information was retained for all 170 diseases. The generated model was then used to evaluate the performance of the model by predicting missing drug targets associated with the one disease mentioned above. This evaluation was repeated for all the 170 diseases.

The evaluation of the prediction performance of each disease was based on AUC scores.

The AUC scores were calculated as follows. Each biofeature was ranked according to sampling frequency with 1 being the highest and 0 being the lowest. This ranking score for each biofeature was compared to the score of 1 for known (observed) biofeatures and the score of 0 for all the other biofeatures (i.e., unobserved biofeatures). The AUC scores were calculated for each type of feature by comparing the scores of all sampled biofeature groups of each type.

FIG. 5a shows results. When predicting the missing drug targets, 104 diseases of the 170 diseases had AUC scores greater than 0.7 and 81 diseases had AUC scores greater than 0.8. In contrast, when predicting the missing SNPs, only 4 diseases had AUC scores greater than 0.7. This result was considered to suggest that the number of SNPs related to each disease was very high.

The same approach was then used to evaluate the performance of a second model, the microbiota/drug target model (i.e., the “type” in the above description of the extended LDA model is “microbiota and drug target”) (FIG. 5b, Listing 2). The target diseases are 104 diseases with both microbiota and drug target biofeatures.

FIG. 5b shows results. In the second model, when predicting the missing microbiota, 61 diseases had AUC scores greater than 0.7 and 39 diseases had AUC scores greater than 0.8. When predicting the missing drug targets, 48 diseases had AUC scores greater than 0.7 and 24 diseases had AUC scores greater than 0.8.

These results indicate that the extended LDA model can predict omics data associated with missing diseases.

### 3-2. Prediction of Putative Biomarkers and Therapeutic Targets

The framework for predicting disease-associated biomarkers and/or therapeutic targets from the missing biofeatures predicted by the extended LDA model was applied to disease-associated tissue/organ transcriptome data. The drug target data, microbiota data, and disease-associated tissue/organ transcriptome data were combined and applied to the extended LDA model. Out of the 104 diseases with drug targets and microbiota features, 25 diseases were also tagged with tissue/organ transcriptome features. Thus, transcriptome information was missing for 79 diseases. That is, the missing transcriptome information for these 79 diseases is the transcriptome information newly associated with each disease. Therefore, it is suggested that the transcriptome corresponding to the missing transcriptome information may serve as a novel biomarker and/or therapeutic target for the above 79 diseases.

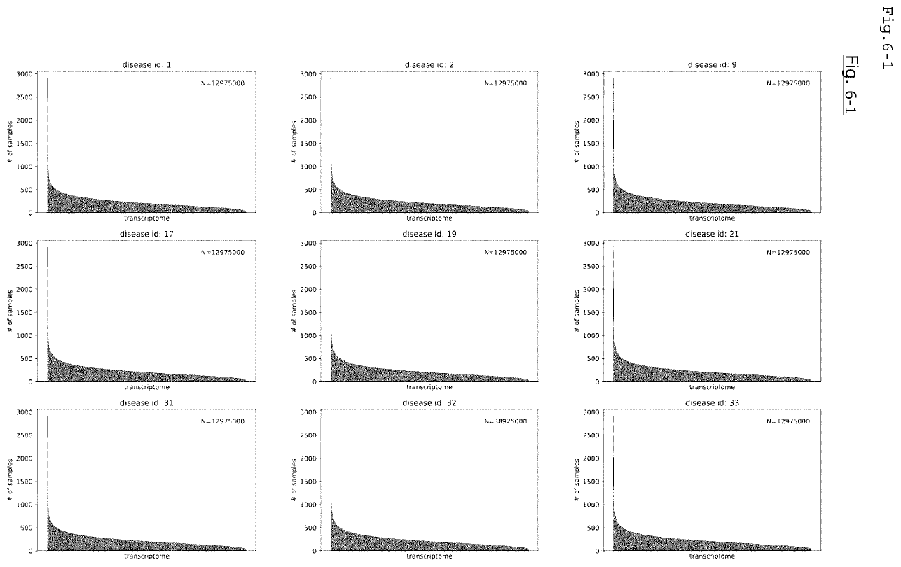

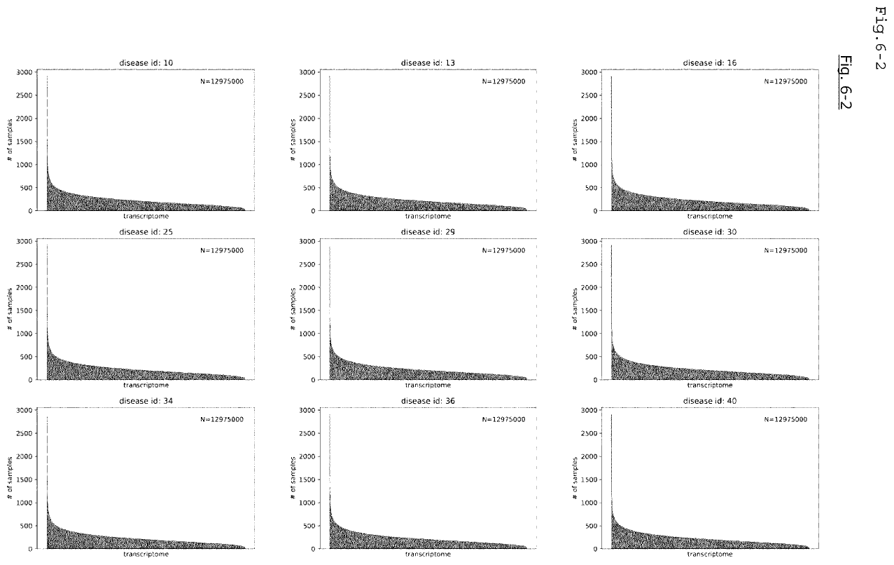

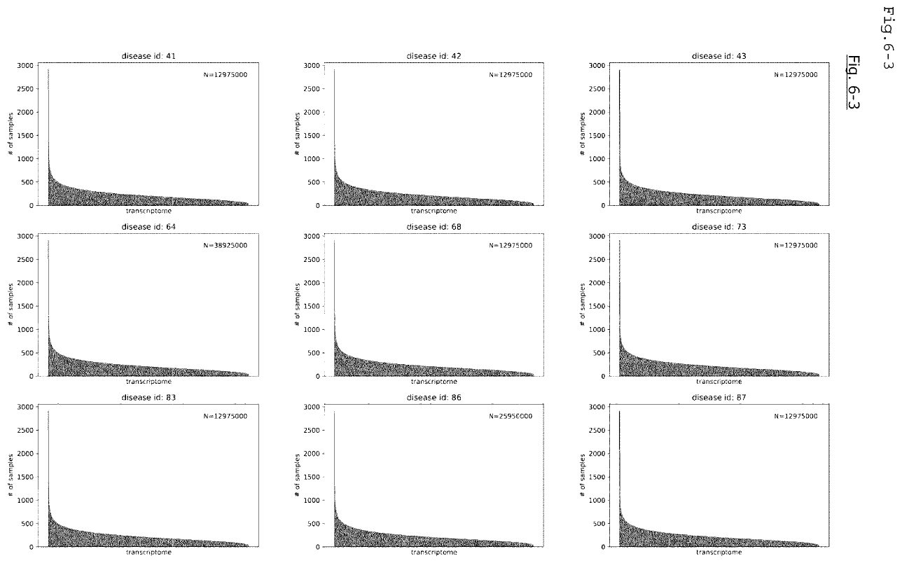

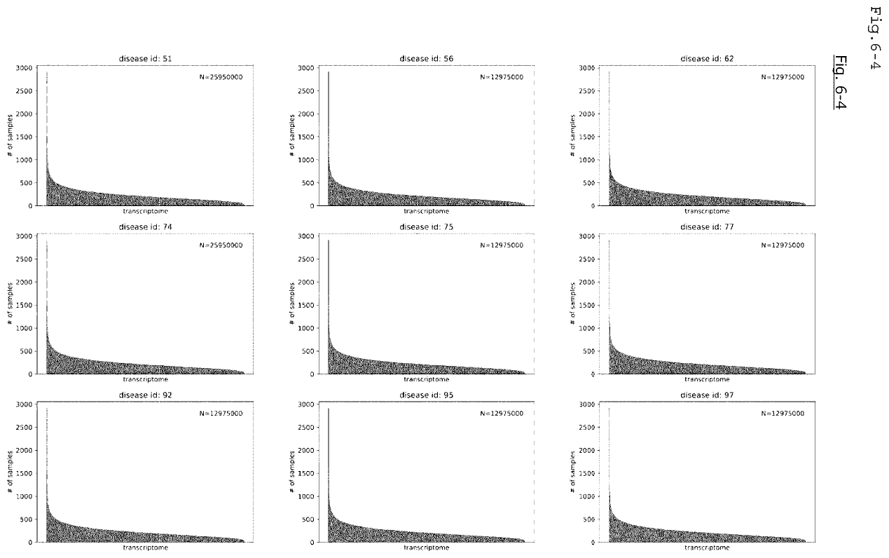

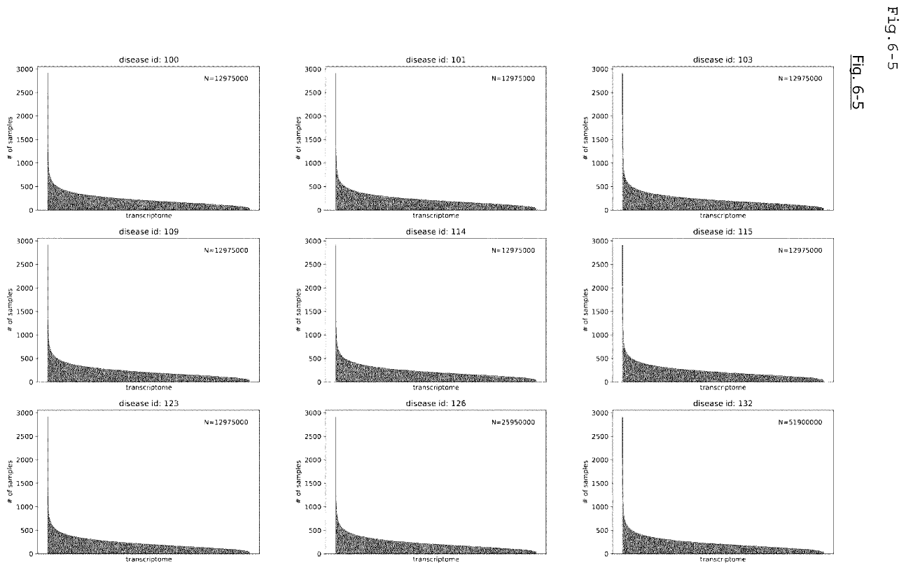

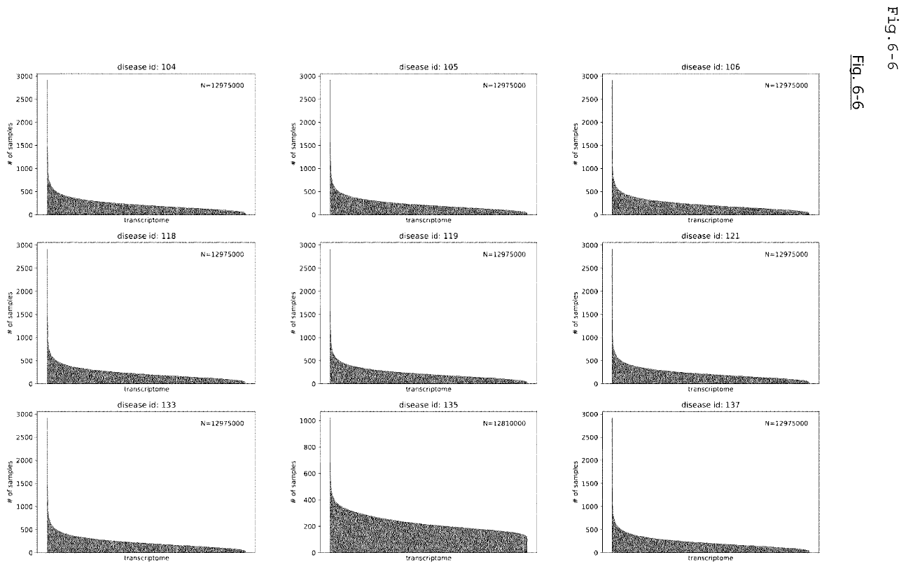

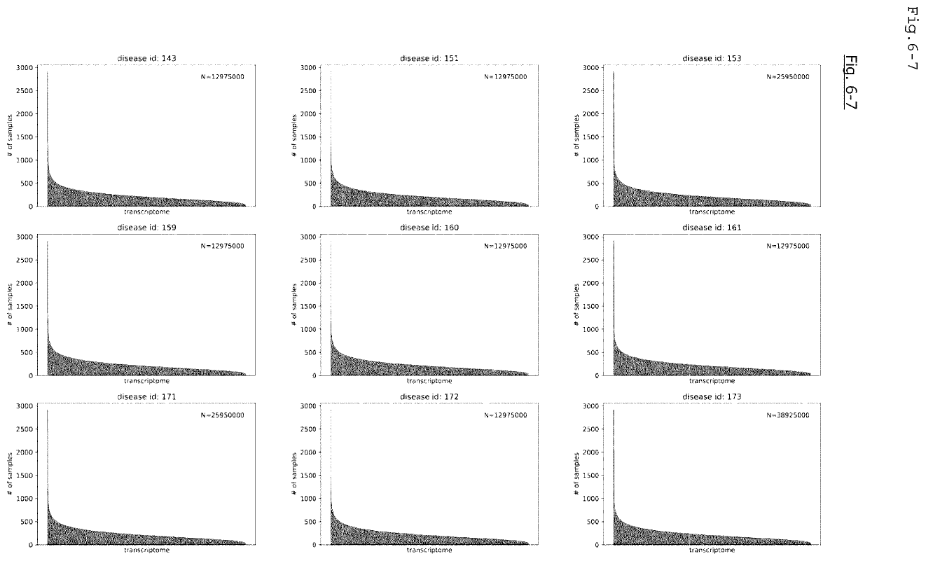

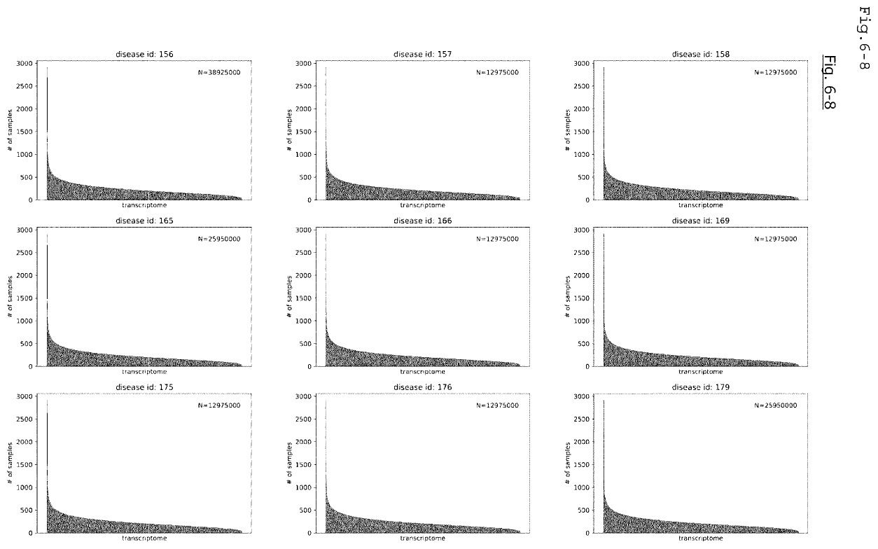

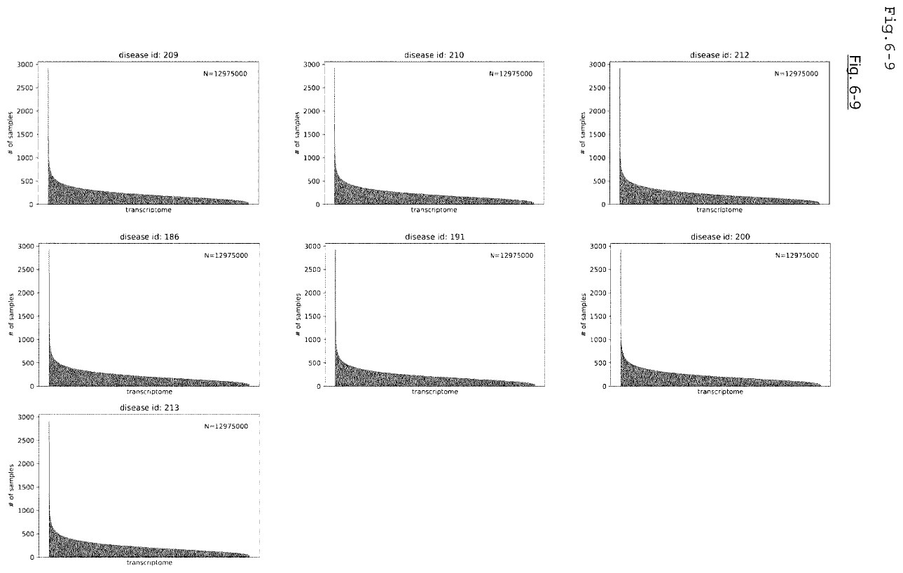

Analysis of the above 79 diseases identified numerous tissue/organ genes for each disease (FIG. 6, <Listing 3: Novel markers> at the end of the present description).

In particular, some tissue/organ genes showed significantly higher counts (1,018 to 2,911 counts) than other tissue/organ genes (FIG. 6, Listing 3). This approach identified about 1 to 20 tissue/organ genes highly associated with each of the 79 diseases (FIG. 6, Listing 3). Most of them were organs found throughout the body (blood, immune cells) (Listing 3). It was shown to be able to be used as the candidate for biomarkers and/or therapeutic targets for predicting diseases linked by word analysis with the extended LDA model.

### Supporting Information

### <Listing 1: GEO Datasets>

Data are listed in the order (UMLS ID, GSE, Ctrl_num,Disease_num, Disease, Tissue_origin, Tissue_rough_class, Note). The “Ctrl_num” is the number of controls, the “Disease_num” is the number of diseases, the “Disease” is the name of a disease, the “Tissue_origin” is the name of a tissue or organ, the “Tissue_rough_class” is the name of a tissue when manually classified, and the “Note” is a note.
(C0003165|C0014013|C0032273|C0032274|C0037116|C0041296|C0041300|C0041301|C004131 8|C0041321|C0041322|C0041324|C0041330|C0041333|C0152717|C0152861|C0152874|C01528 89|C0152915|C0153333|C0153334|C0156756|C0260341|C0260867|C0275716|C0275887|C0343 426|C0348102|C0348103|C0348104|C0348105|C0348106|C0348107|C0348109|C0348301|C034 8466|C0348513|C0348532|C0348539|C0348810|C034881|C0348812|C03 48813|C0348825|C0348960|C0348961|C0348962|C0348963|C0348964|C0348966|C0348967|C0 348968|C0348969|C0477446|C0477650|C0477749|C0477767|C0477769|C0477773|C0494034|C 0494035|C0494036|C0494037|C0494132|C0494662|C0496581|C0496612|C0694497|C1331984|C1331985|C1533626|C3203357,GSE148036,5,5,Tuberculosis,Lung,Lung)
(C0003165|C0014013|C0032273|C0032274|C0037116|C0041296|C0041300|C0041301|C004131 8|C0041321|C0041322|C0041324|C0041330|C0041333|C0152717|C0152861|C0152874|C01528 89|C0152915|C0153333|C0153334|C0156756|C0260341|C0260867|C0275716|C0275887|C0343 426|C0348102|C0348103|C0348104|C0348105|C0348106|C0348107|C0348109|C0348301|C034 8466|C0348513|C0348532|C0348539|C0348810|C0348811|C0348812|C03 48813|C0348825|C0348960|C0348961|C0348962|C0348963|C0348964|C0348966|C0348967|C0 348968|C0348969|C0477446|C0477650|C0477749|C0477767|C0477769|C0477773|C0494034|C 0494035|C0494036|C0494037|C0494132|C0494662|C0496581|C0496612|C0694497|C1331984|C1331985|C1533626|C3203357,GSE131031,4,20,Tuberculosis,ILC2,Lymphocyte,ILC2 from Lung)
(C0003165|C0014013|C0032273|C0032274|C0037116|C0041296|C0041300|C0041301|C004131 8|C0041321|C0041322|C0041324|C0041330|C0041333|C0152717|C0152861|C0152874|C01528 89|C0152915|C0153333|C0153334|C0156756|C0260341|C0260867|C0275716|C0275887|C0343 426|C0348102|C0348103|C0348104|C0348105|C0348106|C0348107|C0348109|C0348301|C034 8466|C0348513|C0348532|C0348539|C0348808|C0348809|C0348810|C0348811|C0348812|C03 48813|C0348825|C0348960|C0348961|C0348962|C0348963|C0348964|C0348966|C0348967|C0 348968|C0348969|C0477446|C0477650|C0477749|C0477767|C0477769|C0477773|C0494034|C 0494035|C0494036|C04940371|C0494132|C0494662|C0496581|C0496612|C06944971|C1331984|C1331985|C1533626|C3203357,GSE114192,36,44,Tuberculosis (TB) and Intermediate hyperglycemia (IH),Whole_blood,Whole_blood)
(C0003165|C0014013|C0032273|C0032274|C0037116|C0041296|C00413001|C0041301|C004131 8|C0041321|C0041322|C0041324|C0041330|C0041333|C0152717|C0152861|C0152874|C01528 89|C0152915|C0153333|C0153334|C0156756|C0260341|C0260867|C0275716|C0275887|C0343 426|C0348102|C0348103|C0348104|C0348105|C0348106|C0348107|C0348109|C0348301|C034 8466|C0348513|C0348532|C0348539|C0348808|C0348809|C0348810|C034881|C0348812|C03 48813|C0348825|C0348960|C0348961|C0348962|C0348963|C0348964|C0348966|C0348967|C0 348968|C0348969|C0477446|C0477650|C0477749|C0477767|C0477769|C0477773|C0494034|C 0494035|C0494036|C0494037|C0494132|C0494662|C0496581|C0496612|C0694497|C1331984| C1331985|C1533626|C3203357,GSE114192,36,61,Tuberculosis (TB) and Diabetes mellitus (DM),Whole_blood,Whole_blood)
(C0003165|C0014013|C0032273|C0032274|C0037116|C0041296|C00413001|C0041301|C004131 8|C0041321|C0041322|C0041324|C0041330|C0041333|C0152717|C0152861|C0152874|C01528 89|C0152915|C0153333|C0153334|C0156756|C0260341|C0260867|C0275716|C0275887|C0343 426|C0348102|C0348103|C0348104|C0348105|C0348106|C0348107|C0348109|C0348301|C034 8466|C0348513|C0348532|C0348539|C0348808|C0348809|C0348810|C0348811|C0348812|C03 48813|C0348825|C0348960|C0348961|C0348962|C0348963|C0348964|C0348966|C0348967|C0 348968|C0348969|C0477446|C0477650|C0477749|C0477767|C0477769|C0477773|C0494034|C 0494035|C0494036|C0494037|C0494132|C0494662|C0496581|C0496612|C0694497|C1331984| C1331985|C1533626|C3203357,GSE114192,36,46,Tuberculosis (TB),Whole_blood,Whole_blood)
(C0003872|C0033860|C0263361|C0343052|C0343055|C0409673|C0477485,GSE54456,82,92,P soriasis,Skin,Skin)
(C0003872|C0033860|C0263361|C0343052|C0343055|C0409673|C0477485,GSE41745,3,3,Psor iasis,Skin,Skin)
(C0003872|C0033860|C0263361|C0343052|C0343055|C0409673|C0477485,GSE121212,38,28, Psoriasis,Skin,Skin,Healthy vs. AD lesional skin)
(C0003872|C0033860|C0263361|C0343052|C0343055|C0409673|C0477485,GSE121212,38,27, Psoriasis,Skin,Skin,Healthy vs. AD non-lesional skin)
(C0004096|C0023212|C0038218|C0155880|C0340139|C0348819|C0349268|C04786251|C0494650|C04946591|C04946601|C04946781|C0496984|C0496998,GSE85567,28,57,Asthma,Airway epithelium,Airway epithelium)
(C0004096|C0023212|C0038218|C0155880|C0340139|C0348819|C0349268|C0478625|C049465 0|C0494659|C0494660|C0494678|C0496984|C0496998,GSE109484,14,33,Asthma,Airway epithelium,Airway epithelium)
(C0004352|C0011768|C0025362|C0236792|C0282512|C0338986|C0349348|C0869217,GSE305 73,2,2,Autism,Temporal cortex,Temporal_cortex)
(C0005586|C00105981|C0029232|C0236765|C0349208|C0349210|C0349211|C0349212|C034921 3|C0349214|C0349215|C0349218|C0494396|C2939428,GSE80655,24,24,Bipolar disorder,anterior cingulate cortex (AnCg),anterior cingulate cortex (AnCg))
(C0005586|C00105981|C0029232|C0236765|C0349208|C0349210|C0349211|C0349212|C034921 3|C0349214|C0349215|C0349218|C0494396|C2939428,GSE80336,18,18,Bipolar disorder,Dorsal striatum,Dorsal striatum)
(C0005586|C00105981|C0029232|C0236765|C0349208|C0349210|C0349211|C0349212|C034921 3|C0349214|C0349215|C0349218|C0494396|C2939428,GSE112523,17,10,Bipolar disorder,Frontal cortex,Frontal_cortex)
(C0005586|C00105981|C0029232|C0236765|C0349208|C0349210|C0349211|C0349212|C034921 3|C0349214|C0349215|C0349218|C0494396|C2939428,GSE80655,22,24,Bipolar disorder,nucleus accumbens (nAcc),nucleus accumbens (nAcc))
(C0005586|C00105981|C0029232|C0236765|C0349208|C0349210|C0349211|C0349212|C034921 3|C0349214|C0349215|C0349218|C0494396|C2939428,GSE80655,24,23,Bipolar disorder,dorsolateral prefrontal cortex (DLPFC),Prefrontal_cortex)
(C0005586|C00105981|C0029232|C0236765|C0349208|C0349210|C0349211|C0349212|C034921 3|C0349214|C0349215|C0349218|C0494396|C2939428,GSE42546,29,16,Bipolar disorder,dentate gyrus (DG) granule cells in postmortem hippocampus,Hippocampus)
(C0009324|C0010346|C01519712|C03487372|C0349268|C0400832|C0409696|C0451837,GSE112 057,12,15,Ulcerative colitis,Whole_blood,Whole_blood)
(C0009324|C0010346|C0151971|C0348737|C0349268|C0400832|C0409696|C0451837,GSE836 87,8,1,Ulcerative Colitis,Small bowel,Small intestine)
(C0009324|C0010346|C0151971|C0348737|C0349268|C0400832|C0409696|C0451837,GSE836 87,11,1,Ulcerative Colitis,Sigmoid,Sigmoid)
(C0009324|C0010346|C0151971|C0348737|C0349268|C0400832|C0409696|C0451837,GSE836 87,17,5,Ulcerative Colitis,right colon,Colon)
(C0009324|C0010346|C0151971|C0348737|C0349268|C0400832|C0409696|C0451837,GSE836 87,2,1,Ulcerative Colitis,Ileum,Ileum)
(C0009324|C0010346|C0151971|C0348737|C0349268|C0400832|C0409696|C0451837,GSE836 87,5,21,Ulcerative Colitis,left colon,Colon)
(C0009324|C0010346|C0151971|C0348737|C0349268|C04008321|C0409696|C0451837,GSE137 344,37,44,Ulcerative Colitis,Ileum,Ileum)
(C0009324|C0010346|C0151971|C0348737|C0349268|C0400832|C0409696|C0451837,GSE836 87,2,3,Ulcerative Colitis,Rectum,Rectum)
(C0010674|C0270246|C0348692|C0348815|C0348816|C0494350|C2939175,GSE136371,16,33, Cystic fibrosis,Whole_blood,Whole_blood)
(C00011251|C0016142|C0029225|C0029226|C0030477|C0036337|C0036339|C0036341|C003634 4|C0036347|C0036349|C0036351|C0149654|C0221520|C0236818|C0270497|C0338650|C03387 98|C0349192|C0349193|C0349194|C0349195|C0349196|C0349199|C0349200|C0349203|C0349 243|C0349277|C03497091|C0392322|C0494408|C1263846|C1443045,GSE107638,27,23,Schizop hrenia,Brain_Neuronal (from Brodmann area 46),Brain_Neuronal)
(C00011251|C0016142|C0029225|C0029226|C0030477|C0036337|C0036339|C0036341|C003634 4|C0036347|C0036349|C0036351|C0149654|C0221520|C0236818|C0270497|C0338650|C03387 98|C0349192|C0349193|C0349194|C0349195|C0349196|C0349199|C0349200|C0349203|C0349 243|C0349277|C0349709|C0392322|C0494408|C1263846|C1443045,GSE107638,22,17,Schizop hrenia,Brain_Oligodendrocyte (from Brodmann area 46),Brain_Oligodendrocyte)
(C00011251|C0016142|C0029225|C0029226|C0030477|C0036337|C0036339|C0036341|C003634 4|C0036347|C0036349|C0036351|C0149654|C0221520|C0236818|C0270497|C0338650|C03387 98|C0349192|C0349193|C0349194|C0349195|C0349196|C0349199|C0349200|C0349203|C0349 243|C0349277|C03497091|C0392322|C0494408|C1263846|C1443045,GSE107638,4,3,Schizophr enia,Brain_Cytoplasmic_fraction (from Brodmann area 46),Brain Cytoplasmic_fraction)
(C00011251|C0016142|C0029225|C0029226|C0030477|C0036337|C0036339|C0036341|C003634 4|C0036347|C0036349|C0036351|C0149654|C0221520|C0236818|C0270497|C0338650|C03387 98|C0349192|C0349193|C0349194|C0349195|C0349196|C0349199|C0349200|C0349203|C0349 243|C0349277|C0349709|C0392322|C0494408|C1263846|C1443045,GSE112523,17,7,Schizoph renia,Frontal cortex,Frontal_cortex)
(C00011251|C0016142|C0029225|C0029226|C0030477|C0036337|C0036339|C0036341|C003634 4|C0036347|C0036349|C0036351|C0149654|C0221520|C0236818|C0270497|C0338650|C03387 98|C0349192|C0349193|C0349194|C0349195|C0349196|C0349199|C0349200|C0349203|C0349 243|C0349277|C0349709|C0392322|C0494408|C1263846|C1443045,GSE138082,13,7,Schizoph renia,Hippocampus (CA1),Hippocampus)
(C00011251|C0016142|C0029225|C0029226|C0030477|C0036337|C0036339|C0036341|C003634 4|C0036347|C0036349|C0036351|C0149654|C0221520|C0236818|C0270497|C0338650|C03387 98| C03492771|C03497091|C0392322| C04944081|C1263 846| C1443045,GSE 13 8082,13,7, Schizoph renia,Hippocampus (CA3),Hippocampus)
(C0011251|C0016142|C0029225|C0029226|C0030477|C0036337|C0036339|C0036341|C003634 4|C0036347|C0036349|C0036351|C0149654|C0221520|C0236818|C0270497|C0338650|C03387 98|C0349192|C0349193|C0349194|C0349195|C0349196|C0349199|C0349200|C0349203|C0349 243|C0349277|C0349709|C0392322|C0494408|C1263846|C1443045,GSE42546,29,17,Schizoph renia,dentate gyrus (DG) granule cells in postmortem hippocampus,Hippocampus)
(C00011251|C0016142|C0029225|C0029226|C0030477|C0036337|C0036339|C0036341|C003634 4|C0036347|C0036349|C0036351|C0149654|C0221520|C0236818|C0270497|C0338650|C03387 98|C0349192|C0349193|C0349194|C0349195|C0349196|C0349199|C0349200|C0349203|C0349 243|C0349277|C0349709|C0392322|C0494408|C1263846|C1443045,GSE80655,24,24,Schizoph renia,anterior cingulate cortex (AnCg),anterior cingulate cortex (AnCg))
(C00011251|C0016142|C0029225|C0029226|C0030477|C0036337|C0036339|C0036341|C003634 4|C0036347|C0036349|C0036351|C0149654|C0221520|C0236818|C0270497|C0338650|C03387 98|C0349192|C0349193|C0349194|C0349195|C0349196|C0349199|C0349200|C0349203|C0349 243|C0349277|C0349709|C0392322|C0494408|C1263846|C1443045,GSE80655,24,24,Schizoph renia,dorsolateral prefrontal cortex (DLPFC),Prefrontal_cortex)
(C00011251|C0016142|C0029225|C0029226|C0030477|C0036337|C0036339|C0036341|C003634 4|C0036347|C0036349|C0036351|C0149654|C0221520|C0236818|C0270497|C0338650|C03387 98|C0349192|C0349193|C0349194|C0349195|C0349196|C0349199|C0349200|C0349203|C0349 243|C0349277|C0349709|C0392322|C0494408|C1263846|C1443045,GSE138082,13,7,Schizoph renia,Hippocampus (DG),Hippocampus)
(C00011251|C0016142|C0029225|C0029226|C0030477|C0036337|C0036339|C0036341|C003634 4|C0036347|C0036349|C0036351|C0149654|C0221520|C0236818|C0270497|C0338650|C03387 98|C0349192|C0349193|C0349194|C0349195|C0349196|C0349199|C0349200|C0349203|C0349 243|C0349277|C0349709|C0392322|C0494408|C1263846|C1443045,GSE80655,22,23,Schizoph renia,nucleus accumbens (nAcc),nucleus accumbens (nAcc))
(C0011615|C0494831,GSE65832,20,20,Atopic dermatitis,Skin,Skin)
(C0011615|C0494831,GSE121212,38,27,Atopic Dermatitis,Skin,Skin,Healthy vs. AD non-lesional skin)
(C0011615|C0494831,GSE121212,38,21,Atopic Dermatitis,Skin,Skin,Healthy vs. AD lesional skin)
(C0011849|C0011854|C0011860|C0271641|C0348447|C0851208,GSE92724,6,4,Type 2 diabetes (T2D),Dermal blood endothelium,Dermal blood endothelium)
(C0014058|C0026769|C0348564|C0349082|C0494470,GSE138614,25,16,multiple sclerosis,Brain (Active lesion),Brain white matter)
(C0014058|C0026769|C0348564|C0349082|C0494470,GSE138614,25,17,multiple sclerosis,Brain (Chronic active lesion),Brain white matter)
(C0014058|C0026769|C0348564|C0349082|C0494470,GSE138614,25,14,multiple sclerosis,Brain (Inactive lesion),Brain white matter)
(C0014058|C0026769|C0348564|C0349082|C0494470,GSE138614,25,26,multiple sclerosis,Brain (Normal appearing white matter),Brain white matter)
(C00140581|C00267691|C03485641|C0349082|C0494470,GSE77598,3,5,Multiple Sclerosis,Monocyte,Monocyte)
(C0014175|C0014177|C0156344|C0156345|C0156346|C0156347|C0156348|C0341858|C047777 81|C0495096,GSE134056,22,16,Endometriosis,Endometrial tissue,Endometrial tissue)
(C00221041|C0178283|C03389391|C0349268,GSE137344,37,1,Irritable bowel syndrome (IBS),Ileum,Ileum)
(C00224081|C00380191|C0702154,GSE114007,18,20,Osteoarthritis (OA),Knee articular cartilage,Knee_articular cartilage)
(C0024141|C0026848|C0263591|C0343426|C0348600|C0348641|C0349082|C0409974|C040997 6|C0451753|C0477343|C0477411|C0477587|C0477884|C0494697|C0495047,GSE136731,4,7,S ystemic lupus erythematosus (SLE),cDC2 dendritic cells (CD5+),cDC2 dendritic cells)
(C0024141|C0026848|C0263591|C0343426|C0348600|C0348641|C0349082|C0409974|C040997 6|C0451753|C0477343|C047741|C04775871|C04778841|C04946971|C0495047,GSE139358,11,11, Systemic lupus erythematosus (SLE),Normal density neutrophil,Neutrophil)
(C0024141|C0026848|C0263591|C0343426|C0348600|C0348641|C0349082|C0409974|C040997 6|C0451753|C0477343|C047741|C04775871|C04778841|C04946971|C0495047,GSE136731,10,7, Systemic lupus erythematosus (SLE),cDC2 dendritic cells (CD5-CD163+CD14−),cDC2 dendritic cells)
(C0024141|C0026848|C0263591|C0343426|C0348600|C0348641|C0349082|C0409974|C040997 6|C0451753|C0477343|C0477411|C0477587|C0477884|C0494697|C0495047,GSE136731,2,1,S ystemic lupus erythematosus (SLE),cDC2 dendritic cells (CD5-CD163−),cDC2 dendritic cells)
(C0024141|C0026848|C0263591|C0343426|C0348600|C0348641|C0349082|C0409974|C040997 6|C0451753|C0477343|C0477411|C0477587|C0477884|C0494697|C0495047,GSE131525,9,9,5 ystemic lupus erythematosus (SLE),CD8+ T cells,T_cells)
(C0024141|C0026848|C0263591|C0343426|C0348600|C0348641|C0349082|C0409974|C040997 6|C0451753|C0477343|C047741|C04775871|C04778841|C04946971|C0495047,GSE122459,6,20, Systemic lupus erythematosus (SLE),PBMC,PBMC)
(C0024141|C0026848|C0263591|C0343426|C0348600|C0348641|C0349082|C0409974|C040997 6|C0451753|C0477343|C047741|C04775871|C04778841|C04946971|C0495047,GSE131525,9,9,5 ystemic lupus erythematosus (SLE),B cells,B_cells)
(C0024141|C0026848|C0263591|C0343426|C0348600|C0348641|C0349082|C0409974|C040997 6|C0451753|C0477343|C047741|C04775871|C04778841|C04946971|C0495047,GSE131525,9,9,5 ystemic lupus erythematosus (SLE),Monocyte,Monocyte)
(C0024141|C0026848|C0263591|C0343426|C0348600|C0348641|C0349082|C0409974|C040997 6|C0451753|C0477343|C047741|C04775871|C04778841|C04946971|C0495047,GSE72509,18,99, Systemic lupus erythematosus (SLE) (anti-ro: high/ISM high),Whole_blood,Whole_blood)
(C0024141|C0026848|C0263591|C0343426|C0348600|C0348641|C0349082|C0409974|C040997 6|C0451753|C0477343|C047741|C04775871|C04778841|C04946971|C0495047,GSE118256,8,9,S ystemic lupus erythematosus (SLE),Transitional 3 B cells (T3),Transitional 3 B cells (T3))
(C0024141|C0026848|C0263591|C0343426|C0348600|C0348641|C0349082|C0409974|C040997 6|C0451753|C0477343|C047741|C04775871|C04778841|C04946971|C0495047,GSE118256,8,9,S ystemic lupus erythematosus (SLE),Switched Memory B cells (SM),Switched Memory B cells (SM))
(C0024141|C0026848|C0263591|C0343426|C0348600|C0348641|C0349082|C0409974|C040997 6|C0451753|C0477343|C047741|C04775871|C04778841|C04946971|C0495047,GSE118256,6,9,S ystemic lupus erythematosus (SLE),Resting Naive Bcells,Resting Naive Bcells)
(C0024141|C0026848|C0263591|C0343426|C0348600|C0348641|C0349082|C0409974|C040997 6|C0451753|C0477343|C047741|C04775871|C04778841|C04946971|C0495047,GSE118256,6,8,S ystemic lupus erythematosus (SLE),Double negative B cells,Double negative B cells)
(C0024141|C0026848|C0263591|C0343426|C0348600|C0348641|C0349082|C0409974|C040997 6|C0451753|C0477343|C047741|C04775871|C04778841|C04946971|C0495047,GSE118256,3,4,S ystemic lupus erythematosus (SLE),Antigen Secreting Cell (ASC),Antigen Secreting Cell (ASC))
(C0024141|C0026848|C0263591|C0343426|C0348600|C0348641|C0349082|C0409974|C040997 6|C0451753|C0477343|C047741|C04775871|C04778841|C04946971|C0495047,GSE118256,6,7,S ystemic lupus erythematosus (SLE),Activated Naive B cells,Activated Naive B cells)
(C0024141|C0026848|C0263591|C0343426|C0348600|C0348641|C0349082|C0409974|C040997 6|C0451753|C0477343|C047741|C04775871|C04778841|C04946971|C0495047,GSE131525,9,9,S ystemic lupus erythematosus (SLE),CD4+ T cell,T_cells)
(C0027651,GSE121842,2,2,Rectum cancer,Rectum,Rectum,Pericarcinoma vs. Cancer)
(C0027651,GSE97239,3,3,Bladder cancer,Bladder,Bladder)
(C0027651,GSE133624,29,36,Bladder cancer,Bladder,Bladder,Paracancerous tissue vs. cancer tissue)
(C0028754|C00318808|C01542691|C0260823|C0332544|C0348480|C0348956|C0349252|C045181 9,GSE152991,11,20,Metabolically unhealthy Obese (MUO),Subcutaneous adipose tissue,Adipose tissue,vs. metabolically healthy lean (MHL) (a) MHL (n=11, 6 women) had BMI 18.5-24.9 kg/m2, plasma TG concentration<150 mg/dL, fasting plasma glucose concentration<100 mg/dL, 2-hour OGTT plasma glucose concentration<140 mg/dL, HbAlc 5.6%, and IHTG content <4%; (b) MHO (n=15, 13 women) had BMI 30-49.9 kg/m2, plasma TG concentration <150 mg/dL, fasting plasma glucose concentration <100 mg/dL, 2-hour OGTT plasma glucose concentration <140 mg/dL, HbAlc <5.6%, and IHTG content <4%; and (c) MUO (n=20, 15 women) had BMI 30-49.9 kg/m2, prediabetes (fasting plasma glucose concentration ≥100 mg/dL, 2-hour OGTT plasma glucose concentration ≥140 mg/dL, and/or HbAlc ≥5.7%), and IHTG content ≥5%.)
(C0028754|C00318808|C01542691|C0260823|C0332544|C0348480|C0348956|C0349252|C045181 9,GSE152991,11,14,Metabolically healthy Obese (MHO),Subcutaneous adipose tissue,Adipose tissue,vs. metabolically healthy lean (MHL) (a) MHL (n=11, 6 women) had BMI 18.5-24.9 kg/m2, plasma TG concentration <150 mg/dL, fasting plasma glucose concentration <100 mg/dL, 2-hour OGTT plasma glucose concentration <140 mg/dL, HbAlc 5.6%, and IHTG content <4%; (b) MHO (n=15, 13 women) had BMI 30-49.9 kg/m2, plasma TG concentration <150 mg/dL, fasting plasma glucose concentration <100 mg/dL, 2-hour OGTT plasma glucose concentration <140 mg/dL, HbAlc 5.6%, and IHTG content <4%; and (c) MUO (n=20, 15 women) had BMI 30-49.9 kg/m2, prediabetes (fasting plasma glucose concentration 100 mg/dL, 2-hour OGTT plasma glucose concentration ≥140 mg/dL, and/or HbAlc 5.7%), and IHTG content 5%.)
(C0036220|C0153464|C0153519|C0153538|C0153560|C0153561|C0153562|C0153565|C027653 5|C0348355|C0348364,GSE100684,4,4,Kaposi sarcoma,Unknown,Unknown)
(C0036421|C0349082|C0451842|C0477589|C0494697,GSE136731,4,4,Systemic sclerosis,cDC2 dendritic cells (CD5+),cDC2 dendritic cells)
(C0036421|C0349082|C0451842|C0477589|C0494697,GSE136731,10,5,Systemic sclerosis,cDC2 dendritic cells (CD5-CD163+CD14−),cDC2 dendritic cells)
(C0085084,GSE124439,4,37,ALS (Amyotrophic lateral sclerosis) spectrum MND (Motor neurone diseases),Motor cortex (lateral),Motor cortex)
(C0085084,GSE124439,4,43,ALS (Amyotrophic lateral sclerosis) spectrum MND (Motor neurone diseases),Motor cortex (medial),Motor cortex)
(C0085084,GSE124439,18,64,ALS (Amyotrophic lateral sclerosis) spectrum MND (Motor neurone diseases),Frontal cortex,Frontal cortex)
(C0085084,GSE122649,12,4,Sporadic amyotrophic lateral sclerosis (sALS) and Frontotemporal dementia (FTD),Motor cortex,Motor cortex)
(C0085084,GSE122649,12,22,Sporadic amyotrophic lateral sclerosis (sALS),Motor cortex,Motor cortex)
(C0348703,GSE79544,7,16,Idiopathic Pulmonary Fibrosis (IPF),lung bronchoalveolar lavage(BAL) macrophage,Macrophage)
(C0348703,GSE73189,5,4,Idiopathic Pulmonary Fibrosis (IPF),Lung,Lung)
(C0348703,GSE138283,5,12,Idiopathic Pulmonary Fibrosis (IPF),Lung,Lung)
(C0348703,GSE52463,7,8,Idiopathic Pulmonary Fibrosis (IPF),Lung,Lung)
(breast cancer,GSE110114,3,10,Breast cancer (Luminal B type),Breast,Breast)
(breast cancer,GSE117970,50,4,Breast cancer (DCIS; Ductal Carcinoma in Situ),Monocyte,Monocyte)
(breast cancer,GSE117970,50,29,Breast cancer (Invasive breast cancer),Monocyte,Monocyte)
(breast cancer,GSE124030,1,6,Breast cancer,Breast,Breast)
(breast cancer,GSE117970,4,4,Breast cancer (Invasive breast cancer),Macrophage,Macrophage)
(colorectal cancer,GSE50760,18,18,metastatic colorectal cancer to the liver (stage IV),Colorectum,Colorectum)
(colorectal cancer,GSE109203,3,4,Colorectal tumor (poorly differentiated),Colorectal tissue,Colorectum)
(colorectal cancer,GSE109203,3,4,Colorectal tumor (well differentiated),Colorectal tissue,Colorectum)
(colorectal cancer,GSE109203,3,4,Colorectal tumor (moderately differentiated),Colorectal tissue,Colorectum)
(diabetic nephropathy,GSE142025,9,6,Early Diabetic Nephropathy (DN),Kidney,Kidney)
(endometrial cancer,GSE117970,50,4,Endometrial cancer (UPSC; Uterine papillary serous carcinoma),Monocyte,Monocyte)
(endometrial cancer,GSE117970,5,1,Endometrial cancer (Carcinosarcoma),Macrophage,Macrophage)
(endometrial cancer,GSE117970,5,5,Endometrial cancer (Endometrioid),Macrophage,Macrophage)
(endometrial cancer,GSE56087,9,9,Endometrial tumor,Endometrial tissue,Endometrial tissue,Control: adjacent normal tissue)
(endometrial cancer,GSE146889,10,11,Endometrial tumor,Endometrial tissue,Endometrial tissue,MSI-H, likely Lynch due to MSH6 germline)
(endometrial cancer,GSE146889,3,4,Endometrial tumor,Endometrial tissue,Endometrial tissue,MSI-H, likely Lynch due to PMS2 germline)
(endometrial cancer,GSE146889,10,9,Endometrial tumor,Endometrial tissue,Endometrial tissue,MSI-H, MLH1 hypermethylation)
(endometrial cancer,GSE117970,50,5,Endometrial cancer (Endometrioid),Monocyte,Monocyte)
(endometrial cancer,GSE117970,5,3,Endometrial cancer (UPSC; Uterine papillary serous carcinoma),Macrophage,Macrophage)
(endometrial cancer,GSE146889,9,9,Endometrial tumor,Endometrial tissue,Endometrial tissue,MSS)
(endometrial cancer,GSE117970,50,1,Endometrial cancer (Carcinoma),Monocyte,Monocyte)

### <Listing 2: Disease ID>

Data are listed in the order (Disease ID for figure, UMLS ID, Disease, Drug Target, SNPs, Microbiota, Human Transcriptome, GSE).
(0,C00007271|C0002962|C0003862|C000803|C00134281|C0018681|C0024031|C00295371|C0030 193|C0030196|C0030319|C0031315|C0039494|C0040997|C0151827|C0154466|C0154729|C015 6401|C0159066|C0184567|C0232286|C0232492|C0242429|C0349278|C0349295|C0392361|C04 23729|C0452135|C047648|C0478118|C0478119|C0478148|C0478488|C0478659|C0494408|C0 494479|C0494993|C0495666|C0495669|C0495671|C0677061|C0995154|C1442976,pain,1,1,0,0, )(1,C00001144|C0001145|C0022548|C0029485|C0152249|C0263437|C0263442|C0494863|C070 2166,acne,1,0,1,0); (2,C0001339|C0153094|C0341465|C0341470|C0348760|C0348762,pancreati tis,1,1,1,0,); (3,C0001546,anxiety and major depressive disorder,0,1,0,0,); (3,C0001546,anxiety in major depressive disorder,0,1,0,0,); (3,C0001546,depressed mood,1,0,0,0,); (3,C00001546,subjective well-being,0,1,0,0,); (4,C0001546|C0001818|C0003469|C0013415|C0020604|C0027804|C0028084|C0 028768|C0030319|C0031572|C0038436|C0236748|C0236816|C0236818|C0260822|C0270305|C 0270549|C0338908|C0349194|C0349230|C0349231|C0349232|C0349233|C0349234|C0349257| C0349274|C0349290|C0349335|C0349337|C0478604|C0494432|C0525045|C1263846|C152728 1,anxiety,1,1,0,0,); (5,C0001546|C0001818|C0003469|C0013415|C0020604|C0027804|C002808 4|C0028768|C0030319|C0038436|C0236748|C0236816|C0236818|C0270305|C0270549|C03389 08|C0349194|C0349231|C0349274|C0349290|C0349335|C0349337|C0494432|C1527281,anxiet y
disorder,1,1,0,0,); (6,C0001546|C0003431|C00140891|C0016142|C002912|C00374481|C0149654|C0221520|C0339004|C0339009|C0339010|C0339017|C0349217|C0349335|C0349336|C049443 2|C0579986|C0694453,conduct
disorder,1,1,0,0,); (7,C0001546|C00278041|C0349268,worry,1,1,0,0,); (8,C00018181|C0030319|C0 2367481|C0236816,panic disorder,1,1,0,0,); (9,C0002171|C0477504,alopecia areata,1,1,1,0,); (10,C0003123|C0003125|C0338959|C0494440|C2267227,anorexia nervosa,1,1,1,0,); (11,C0003165|C0014013|C0032273|C0032274|C0037116|C0041296|C0041300|C0041301|C0041318|C0041321|C0041322|C0041324|C0041330|C0041333|C0152717|C015286 1|C0152874|C0152889|C0152915|C0153333|C0153334|C0156756|C0260341|C0260867|C02757 16|C0275887|C0343426|C0348102|C0348103|C0348104|C0348105|C0348106|C0348107|C0348 109|C0348301|C0348466|C0348513|C0348532|C0348539|C03488081|C0348809|C0348810|C034 8811|C0348812|C0348813|C0348825|C0348960|C0348961|C0348962|C0348963|C0348964|C03 48966|C0348967|C0348968|C0348969|C0477446|C0477650|C0477749|C0477767|C0477769|C0 477773|C0494034|C0494035|C0494036|C0494037|C0494132|C0494662|C0496581|C0496612|C 0694497|C1331984|C1331985|C1533626|C3203357,tuberculosis,1,1,1,1,GSE148036, GSE131031); (11,C0003165|C0014013|C0032273|C0032274|C0037116|C0041296|C0041300|C0 041301|C0041318|C0041321|C0041322|C0041324|C0041330|C0041333|C0152717|C0152861|C 0152874|C0152889|C0152915|C0153333|C0153334|C0156756|C0260341|C0260867|C0275716| C0275887|C0343426|C0348102|C0348103|C0348104|C0348105|C0348106|C0348107|C034810 9|C0348301|C0348466|C0348513|C0348532|C0348539|C0348808|C0348809|C0348810|C03488 11|C0348812|C0348813|C0348825|C0348960|C0348961|C0348962|C0348963|C0348964|C0348 966|C0348967|C0348968|C0348969|C0477446|C0477650|C0477749|C0477767|C0477769|C047 7773|C0494034|C0494035|C0494036|C0494037|C0494132|C0494662|C0496581|C0496612|C06 94497|C1331984|C1331985|C1533626|C3203357,tuberculosis (tb),0,0,0,1,GSE114192); (11,C0003165|C0014013|C0032273|C0032274|C0037116|C0041296|C 0041300|C0041301|C0041318|C0041321|C0041322|C0041324|C0041330|C0041333|C0152717| C0152861|C0152874|C0152889|C0152915|C0153333|C0153334|C0156756|C0260341|C026086 7|C0275716|C0275887|C0343426|C0348102|C0348103|C0348104|C0348105|C0348106|C03481 07|C0348109|C0348301|C0348466|C0348513|C0348532|C0348539|C0348808|C0348809|C0348 810|48811|CC0348812|C0348813|C0348825|C0348960|C0348961|C0348962|C0348963|C034 8964|C0348966|C0348967|C0348968|C0348969|C0477446|C0477650|C0477749|C0477767|C04 77769|C0477773|C0494034|C0494035|C0494036|C0494037|C0494132|C0494662|C0496581|C0 496612|C0694497|C1331984|C1331985|C1533626|C3203357,tuberculosis (tb) and diabetes mellitus
(dm),0,0,0,1,GSE114192); (11,C0003165|C0014013|C0032273|C0032274|C0037116|C0041296| C0041300|C0041301|C0041318|C0041321|C0041322|C0041324|C0041330|C0041333|C015271 7|C0152861|C0152874|C0152889|C0152915|C0153333|C0153334|C0156756|C0260341|C02608 67|C0275716|C0275887|C0343426|C0348102|C0348103|C0348104|C0348105|C0348106|C0348 107|C0348109|C0348301|C0348466|C0|C03448539|C0348808|C03488091|C034 8810|C0348811|C0348812|C0348813|C0348825|C0348960|C0348961|C0348962|C0348963|C03 48964|C0348966|C0348967|C0348968|C0348969|C0477446|C0477650|C0477749|C0477767|C0 477769|C0477773|C0494034|C0494035|C0494036|C0494037|C0494132|C0494662|C0496581|C 0496612|C0694497|C1331984|C1331985|C1533626|C3203357,tuberculosis (tb) and intermediate hyperglycemia
(ih),0,0,0,1,GSE114192); (12,C0003431,frustration,1,0,0,0,); (12,C0003431,callous-unemotional behaviour,0,1,0,0,); (13,C0003615|C0031154|C0085693|C0152505|C0156095,appendicitis,1,1,1, 0,); (14,C00038601|C0032533|C03432001|C0477585,giant cell arteritis,1,1,0,0,); (15,C0003872|C0033860|C0263361|C0343052|C0343055|C0409673|C0477485,psoriasis,1,1,1,1,GSE54456, GSE41745, GSE121212, GSE121212); (16,C0003873|C0015773|C0026848|C0029660|C0038013|C0409651|C0409652|C0 477411|C0477541|C0477542|C0494896|C0494897|C0694497|C3714757,rheumatoid arthritis,1,1,1,0,); (17,C0003962|C0036528|C0152717|C0156180|C0477842|C0495216|C0495676|C0495700,ascites,1,0,1,0,); (18,C0004096|C0023212|C0038218|C0155880|C0340139|C0348819| C0349268|C0478625|C04946501|C0494659|C04946601|C0494678|C0496984|C0496998,asthma,1, 1,1,1,GSE85567,
GSE109484); (19,C0004153|C0007775|C0010054|C0017327|C0155733|C0155734|C03486481|C0 494764|C0495064|C0495663|C3495604,atherosclerosis,1,0,1,0,); (20,C0004352| C0011768|C002 5362|C0236792|C0282512|C0338986|C0349348|C0869217,autism,1,1,1,1,GSE30573); (21,C000 46261|C0032285|C0032300|C0032302|C0032308|C0032310|C0034063|C01 15|C2487|C01 52502|C0155858|C0155860|C0155862|C0158935|C0158944|C0206062|C0260334|C0264522|C0 276026|C0276089|C0276156|C0276333|C0339951|C0339959|C0339961|C0339969|C0339971|C 0343320|C0343323|C0343324|C0343666|C0348152|C0348161|C0348193|C0348677|C0348678| C0348679|C0348680|C0348681|C0348684|C0348685|C0348703|C0348801|C0348804|C034880 6|C0348809|C0348814|C0348968|C0348970|C0349359|C0349468|C0375326|C0477908|C04779 09|C0494034|C0494059|C0494086|C0494137|C0494638|C0494639|C0494640|C0494643|C0494 644|C0494675|C0494678|C0494683|C0495389|C0495390|C0519030|C0694504|C0869210,pneu monia,1,1,1,0,); (22,C0004698|C0149938|C0268701|C0268732|C0342611|C0348988|C0451767| C0451769|C04848548|C0868872|C0869068,nephropathy,1,1,0,0, ); (23,C0005586|C0010598|C0013415|C0020604|C0027804|C0030319|C0038436|C0154411|C01 54412|C0154413|C0270268|C0338808|C0338908|C0339017|C0349212|C0349213|C0349216|C0 349217|C0349218|C0349219|C0349231|C0349236|C0349290|C0392322|C0494396|C0494399|C 0494400|C0494432|C0495315|C0496594|C0525045|C0869478,depression,1,1,0,0,); (24,C000558 6|C0010598|C0029232|C0236765|C0349208|C0349210|C0349211|C0349212|C0349213|C03492 141|C0349215|C03492181|C0494396|C2939428,bipolar disorder,1,1,1,1,GSE80655, GSE80336, GSE112523, GSE80655, GSE80655,
GSE42546); (25,C0006267|C0152239|C0348809|C0348968|C0431530|C0494034|C0494659,bro nchiectasis,1,0,1,0,); (26,C0006413|C0019829|C0021071|C0023434|C0023493|C0024301|C0024 305|C0024419|C0079744|C0079748|C0079758|C0079774|C0152266|C0152267|C0152268|C015 3470|C0348387|C0348388|C0348389|C0348390|C0348394|C0348396|C0451750|C0452189|C04 52192| C0494171| C0494172| C12641871|C12641881C12641901|C1266194,lymphoma, 1, 1,0,0,); (27, C0007286,carpal tunnel
syndrome,1,1,0,0,); (28,C0007820|C0011269|C0393560|C0393561|C0393562|C0478654|C04946 13,vascular
dementia,1,1,0,0,); (29,C0007820|C0013537|C0017614|C001880|C0020538|C002054|C002054 5|C0022661|C0031190|C0033845|C0035078|C0085580|C0151744|C0152105|C0152171|C01556 16|C0155711|C0348586|C0348587|C0348752|C0348859|C0349368|C0392682|C0393561|C0452 204|C0495174|C0495176|C0495177|C0495178|C0495180|C0495183|C0848548|C3495403|C364 8968,hypertension,1,1,1,0,); (30,C0007860|C0014719|C0348904|C0477866|C0494054,cervicitis, 1,0,1,0,); (31,C0007871|C0011849|C0011854|C0011860|C0012242|C0012739|C0013537|C00139 27|C0018790|C0018801|C0019693|C0020450|C0020538|C0021361|C0024528|C0027498|C0027 765|C0028962|C0029496|C0029604|C0031154|C0032969|C0032984|C0032987|C0032989|C003 2991|C0032993|C0032994|C0033010|C0034065|C0035078|C0035204|C0036986|C0038843|C00 39070|C0039236|C0080276|C0085207|C0085740|C0152150|C0152151|C0152152|C0152964|C0 155749|C0155774|C0155778|C0155796|C0156373|C0156543|C0156604|C0156677|C0156699|C 0156703|C0156756|C0157535|C0158806|C0158816|C0161754|C0178269|C0178288|C0178298| C0233214|C0242172|C0260550|C0260551|C0263660|C0269294|C0269661|C0269672|C027164 1|C0275667|C0275820|C0302487|C0332544|C0339951|C0340747|C0342044|C0342054|C03420 68|C0348447|C0348786|C0348859|C0348877|C0348881|C0348950|C0349253|C0362068|C0391 989|C0392070|C0405328|C0411164|C0411169|C0451717|C0451783|C0451790|C0451791|C045 51822|C0452164|C0452165|C0475523|C0475578|C0476442|C0476491|C0476550|C0476570|C0 476572|C0476635|C0477322|C0477391|C0477762|C0477808|C0477809|C0477810|C0477811|C 0477812|C0477813|C0477814|C0477815|C0477816|C0477817|C0477819|C0477820|C0477865| C0477876|C0477878|C0785211|C0478618|C0494048|C0494450|C049451 4|C0494598|C0494783|C0495168|C0495169|C0495170|C0495171|C0495172|C0495173|C04951 74|C0495175|C0495176|C0495177|C0495180|C0495184|C0495187|C0495188|C0495189|C0495 190|C0495192|C0495196|C0495197|C0495198|C0495199|C0495224|C0495225|C0495227|C049 5251|C0495292|C0495301|C0495302|C0495304|C0495308|C0495309|C0495310|C0495311|C04 95312|C0495313|C0495314|C0495315|C0495316|C0495317|C0495318|C0495351|C0495699|C0 495706|C0496132|C0496136|C0496158|C0496641|C0496642|C0496643|C0496644|C0496645|C 0496646|C0496693|C0496727|C0694449|C0694451|C0694452|C0694454|C0694456|C0700573| C0728889|C0728936|C0851207|C0851208|C0851209|C0851217|C0869210|C0869272|C087854 4|C1314803|C1367972|C1533618|C1533626|C1704300|C3146283|C3648968,pregnancy,1,0,1,0, ); (32,C0008312,primary biliary cirrhosis,0,1,1,0,); (32,C0008312,primary biliary cholangitis,0,1,1,0,); (32,C0008312,biliary cirrhosis
primary,1,0,0,0,); (33,C0008677|C0024117|C0264222|C0348686|C0348693|C0348817|C034881 8|C0494659,chronic obstructive pulmonary
disease,1,1,1,0,); (34,C0008684|C01559371|C03481601|C04947041|C0494706,gingivitis,1,0,1,0,); (3 5,C0008711|C0009443|C0018621|C0348688|C0348689|C0494650|C0494660|C2607914,allergic rhinitis,0,1,0,0,); (35,C0008711|C0009443|C0018621|C0348688|C0348689|C0494650|C0494660| C2607914,rhinitis
allergic,1,0,0,0,); (36,C0008924|C0008925|C0158646|C0432090|C0432098|C0451896|C0451897|C0477977|C0478022|C0495504|C0495539|C0495540|C0495541|C0495542|C0495543|C049554 5|C0869081|C2981150,cleft palate,1,1,1,0,); (37,C0009088,cluster
headache,1,1,0,0,); (38,C00090881|C0018681|C0033893|C014993|C03492681|C0393745|C04755 23|C0475524|C0475525|C0477374|C0494408|C0494479|C0495121|C0546983|C0869050|C0869 051|C0995154|C1735856,headache,1,1,0,0,); (39,C0009324|C0010346|C0151971|C0348737|C03 492681|C0400832|C0409696|C0451837,ulcerative colitis,0,1,1,1,GSE83687, GSE83687, GSE83687, GSE83687, GSE83687, GSE137344,
GSE83687); (39,C0009324|C0010346|C0151971|C0348737|C0349268|C0400832|C0409696|C04 51837,ulcerative
colitis,0,1,1,1,GSE112057); (39,C0009324|C0010346|C0151971|C0348737|C0349268|C0400832|C0409696|C0451837,colitis ulcerative,1,0,0,0,); (40,C0009806|C0014089|C0278008,constipation,1,0,1,0,); (41,C0010054,cor onary artery disease,1,1,1,0,); (42,C00100541|C0348590,coronary heart disease,1,1,1,0,); (43,C0010346|C0156146|C01561471|C0348736|C0409695|C0451836,crohn disease,1,1,1,0,); (44,C0010674|C0270246|C0348692|C0348815|C0348816|C0494350|C2939175, cystic
fibrosis,1,0,1,1,GSE136371); (45,C0011168|C0032249|C0349249|C0495666,dysphagia,1,1,0,0,); (46,C0011251|C0016142|C0029225|C0029226|C0030477|C0036337|C0036339|C0036341|C003 6344|C0036347|C0036349|C0036351|C0149654|C0221520|C0236818|C0270497|C0338650|C03 38798|C0349192|C0349193|C0349194|C0349195|C0349196|C0349199|C0349200|C0349203|C0 349243|C0349277|C0349709|C0392322|C0494408|C1263846|C1443045,schizophrenia,1,1,1,1,G SE107638, GSE107638, GSE107638, GSE112523, GSE138082, GSE138082, GSE42546, GSE80655, GSE80655, GSE138082,
GSE80655); (47,C0011603|C0011616|C0043194|C0162820|C0162823|C0477474|C0494831|C09 36250,eczema,1,1,0,0,); (48,C0011615|C0494831,dermatitis atopic,1,0,0,0,); (48,C0011615|C0494831,atopic dermatitis,0,1,1,1,GSE121212, GSE121212); (48,C0011615|C0494831,atopic dermatitis,0,1,1,1,GSE65832); (49,C00011768|C0014544|C0020703|C0025362|C0027066|C00292 25|C0029233|C0036341|C0236795|C0236826|C0270850|C0282512|C0349082|C0475521|C0477 370|C047737|C0478623|C049447|C0494472|C0495698,epilepsy,1,1,0,0,); (50,C0011849|C001 1854|C0011860|C0032969|C0085207|C0158981|C0260526|C0260823|C0270221|C0271641|C03 484471|C0477716|C0478038|C0478546|C0494336|C0494882|C0495314|C0495663|C0495706|C0 701147|C0851207|C0851208|C08512091|C0851217,diabetes
mellitus,1,1,0,0,); (51,C00011849|C0011854|C0011860|C0271641|C0348447|C0851207,type 1 diabetes,0,1,1,0,); (51,C00011849|C0011854|C0011860|C0271641|C0348447|C0851207,type 1 diabetes
mellitus,1,0,0,0,); (52,C00011849|C0011854|C0011860|C0271641|C0348447|C0851208,type 2 diabetes,0,1,1,0,); (52,C00011849|C0011854|C0011860|C0271641|C0348447|C0851208,type 2 diabetes
(t2d),0,0,0,1,GSE92724); (52,C0011849|C0011854|C0011860|C0271641|C0348447|C0851208,ty pe 2 diabetes mellitus,1,0,0,0,); (53,C00011884,diabetic retinopathy,1,1,0,0,); (54,C0012715|C0154286|C0162316|C0240066|C0476334|C0477300,iron deficiency,1,1,0,0,); (55,C0013295|C0151971,duodenal ulcer,1,1,0,0,); (56,C0013395|C0018834|C0232492,functional dyspepsia,1,0,1,0,); (57,C0013923|C0013924|C0029785|C0034065|C0042374|C0154685|C01557 31|C0155749|C0155755|C0155770|C0155773|C0155774|C0155775|C0155776|C0155777|C0156 194|C0157299|C0157535|C0340325|C0340579|C0340589|C0340712|C0342039|C0342044|C034 8650|C0348786|C0348787|C0348832|C0348876|C0393560|C0451674|C0451676|C0478488|C04 9445001494601|C0494609|C0494611|C0494619|C494620|C0494623|C0494763|C04950641|C0 495184|C0495316,thrombosis,1,1,0,0,); (58,C0014058|C0026769|C0348564|C0349082|C049447 0,multiple sclerosis,1,1,1,1,GSE77598); (58,C0014058|C0026769|C0348564|C0349082|C0494470,multiple sclerosis,1,1,1,1,GSE138614, GSE138614, GSE138614, GSE138614); (59,C0014175|C0014177|C0156344|C0156345|C0156346|C0156347|C0156348|C0 341858|C0477778|C0495096,endometriosis,1,1,1,1,GSE134056); (60,C0014877|C0152210,esotr opia,1,1,0,0,); (61,C0015523,factor xi,0,1,0,0,); (61,C0015523,factor xi deficiency,1,0,0,0,); (62,C0015674|C0024528|C0027804|C0152145|C0236795|C0236816|C04518 80|C0477817|C0546983|C1270972,fatigue,1,0,1,0,); (63,C0017152|C0017154|C0017181|C00383 58|C085695C56|C0156076|C02608238|C0348893|C0694 4641|C2243087,gastritis,1,1,0,0,); (64,C0017154,gastritis atrophic,1,0,0,0,); (64,C0017154,atrophic gastritis,0,0,1,0,); (64,C0017154,gastric atrophy,0,1,0,0,); (65,C0017601|C0017606|C0017614|C0020302|C0036646|C0154777|C0339573|C0348555|C0348557|C0348791|C0391997|C0431451|C0494532|C0494533|C0494534|C049453 5,glaucoma,1,1,0,0,); (66,C0017606,angle closure glaucoma,1,0,0,0,); (66,C0017606,glaucoma (primary angle closure),0,1,0,0,); (67,C018099|C0034139|C0149896|C0409909|C0409910|C0409911|C047754 7|C0494334,gout,1,1,0,0,); (68,C0018681|C0149931|C0338480|C0338483|C0477373|C1735856, migraine,1,1,1,0,); (69,C00187981|C0018816|C00364391|C0477999,congenital heart malformation,0,1,0,0,); (69,C00187981|C0018816|C00364391|C0477999,heart malformation,1,0,0,0,); (70,C00187981|C00364391|C0477999,heart disease congenital,1,0,0,0,); (70,C00187981|C00364391|C0477999,congenital heart disease,0,1,0,0,); (71,C0018800,cardiac hypertrophy,0,1,0,0,); (71,C0018800,cardiomegaly,1,0,0,0,); (72,C0019158|C0019187|C0019189|C0023895|C0042721|C0153075|C0153089|C0276252|C0276434|C0276609|C0276610|C027662 3|C0348185|C0348199|C0348200|C0348305|C0348752|C0400887|C0400895|C0400913|C04009 14|C0400918|C0411016|C0451704|C0451705|C0451709|C0451710|C0451711|C0451712|C0451 800|C476551|01476555|C0477538|C0477937|C0478483|C0489954|C0494092|C0494093|C049 4094|C0494782|C0494783|C0494784|C0494786|C0494787|C0494788|C0494789|C0494790|C04 94794|C0496128|C0524910|C0869060|C1290810|C2063407|C4721555,hepatitis,1,10,0,); (73,C0 0348533|C0348985|C0494081|C0494524,keratitis,1,0,1,0,); (74,C0019693,acquired immunodeficiency syndrome,1,0,1,0,); (74,C0019693,aids,0,1,0,0,); (75,C0020179|C0349080,huntington disease,1,0,1,0,); (76,C0020458,excessive sweating,0,1,0,0,); (76,C0020458,night sweats,1,0,0,0,); (77,C0020517|C0034525|C0156153|C0156154|C0267166|C0348099|C0348101| C0348674|C0348676|C0348738|C0413022|C0494761|C0494762|C0494807|C0496106|C054682 2,gastroenteritis,1,0,1,0,); (78,C0020651|C0039070|C0476454|C0477359,orthostatic hypotension,1,1,0,0,); (79,C0020676|C0342149|C0342151|C0342173|C0342204|C0348442|C034 9082|C0477553|C0494270|C0494271|C0494272|C0494273|C0494364|C0495444|C0495634|C08 69078|C3165526,hypothyroidism,1,1,0,0,); (80,C0021099|C0030807|C0030809|C0038165|C008 5932|C0263312|C0263313|C0263314|C0263316|C0451938|C0477468|C0556023|C2363246,pem phigus,1,1,0,0,); (81,C0021192|C0023343|C0023346|C0023348|C0023351|C0275716|C0343457| C0343458|C034812|C0348302|C0348513|C0477395|C0477639|C0494893,1eprosy,1,1,0,0,); (82,C00213641|C0404584,infertility male,1,0,0,0,); (82,C00213641|C0404584,male infertility,0,1,0,0,); (83,C00213641|C040558|C0476525,azoospermia,1,0,1,0,); (84,C00221041|C01 78283|C03389391|C0349268,irritable bowel syndrome,1,1,1,0,); (84,C00221041|C0178283|C03389391|C0349268,irritable bowel syndrome (ibs),0,0,0,1,GSE137344); (85,C0022408|C0038019|C0702154,osteoarthritis (oa),0,0,0,1,GSE114007); (85,C0022408|C0038019|C0702154,osteoarthritis,1,1,1,0,); (86,C00226 50,kidney stone,0,0,1,0,); (86,C0022650,nephrolithiasis,1,1,0,0,); (87,C0022661|C0041349|C0268731|C034 8642|C0477312|C0495063|C0848548,chronic kidney disease,1,1,1,0,); (88,C0024141|C0026848|C0263591|C0343426|C0348600|C0348641|C0349082| C0409974|C0409976|C0451753|C0477343|C0477411|C0477587|C0477884|C0494697|C049504 7,systemic lupus erythematosus,1,1,1,0,); (88,C0024141|C00268481|C0263591|C0343426|C03486001|C0348641|C0 349082|C0409974|C0409976|C0451753|C0477343|C0477411|C0477587|C0477884|C0494697|C 0495047,systemic lupus erythematosus (sle),0,0,0,1,GSE136731, GSE139358, GSE136731, GSE136731, GSE131525, GSE122459, GSE131525, GSE131525, GSE118256, GSE118256, GSE118256, GSE118256, GSE118256, GSE118256, GSE131525); (88,C0024141|C0026848|C0263591|C0343426|C0348600|C0348641|C0349082|C0 409974|C0409976|C0451753|C0477343|C0477411|C0477587|C0477884|C0494697|C0495047,s ystemic lupus erythematosus (sle) (anti-ro: high/ism high),0,0,0,1,GSE72509); (89,C0024530|C0024535|C0024536|C0024537|C0152072|C0276837|C 0348260|C0348262|C0348263|C0348264|C0348265|C0348986|C0348987|C0348988|C0456106| C0477713|C047792|C0494108|C0869044,malaria,1,1,0,0,); (90,C0024623,gastric cancer,1,1,0,0,); (91,C0025362|C0029233|C049437|C0494428|C1263846|C1270972,cognitive impairment,1,1,0,0,); (92,C0025517|C0263660|C0348791,metabolic syndrome,1,1,1,0,); (93,C0026267,mitral valve prolapse,1,1,0,0,); (94,C0026603,motion sickness,1,1,0,0,); (95,C0026896|C01541991|C0494501|C0495465,myasthenia gravis,1,1,1,0,); (96,C0027430,nasal polyps,1,1,0,0,); (97,C002753|C0037929|C0411063|C0452049|C0452050|C045205|C0478224|C04782571|C0694460|C1331979,spinal cord injury,1,0,1,0,); (98,C0027651,urinary bladder cancer,0,1,0,0,); (98,C0027651,tongue cancer,0,0,1,0,); (98,C0027651,squamous cell carcinoma,1,1,0,0,); (98,C0027651,small cell carcinoma,1,0,0,0,); (98,C0027651,rectum cancer,0,0,0,1,GSE121842); (98,C0027651,pituitary adenoma,1,0,0,0,); (98,C0027651,phaeochromocytoma,1,0,0,0,); (98,C0027651,pancreatic carcinoma,1,0,0,0,); (98,C0027651,pancreatic cancer,0,1,1,0,); (98,C0027651,oral cancer,0,0,1,0,); (98,C0027651,oral cavity cancer,0,1,0,0,); (98,C0027651,metastatic carcinoma,1,0,0,0,); (98,C0027651,metastatic squamous cell carcinoma,1,0,0,0,); (98,C0027651,adrenal adenoma,1,0,0,0,); (98,C0027651,adrenal carcinoma,1,0,0,0,); (98,C0027651,anal cancer,1,0,0,0,); (98,C0027651,biliary tract cancer,0,0,1,0,); (98,C0027651,adenoma benign,1,0,0,0,); (98,C0027651,bone cancer metastatic,1,0,0,0,); (98,C0027651,central nervous system leukaemia,1,0,0,0,); (98,C0027651,lip morphology,0,1,0,0,); (98,C0027651,bladder cancer,1,1,1,1,GSE97239, GSE133624); (99,C0028754|C0031880|C0154269|C0260823|C0332544|C0348480|C0348956|C0 349252|C0451819,metabolically healthy obese (mho),0,0,0,1,GSE152991); (99,C0028754|C0031880|C0154269|C0260823|C0332544|C0348480|C0348956|C0349252|C0451819,metabolically unhealthy obese (muo),0,0,0,1,GSE152991); (99,C0028754|C0031880|C0154269|C0260823|C0332544|C0348480|C0348956|C0349252|C0451819,obesity,1,1,1,0,); (100,C0029456|C00294581|C00296941|C003170 7|C0042870|C0152256|C0158447|C0264115|C0451868|C0451869|C0451870|C0451871|C04518 72|C0451873|C0451881|C0451882|C0451883|C0451884|C0477673|C0477674|C0494808|C0494 958|C04949641|C0494997|C0494998|C0495002|C0495020|C0495120|C0495123|C0521170|C069 4460|C0694523,osteoporosis,1,1,1,0,); (101,C0029882|C0029888|C0152874|C0155415|C015542 1|C0155440|C0155441|C0155460|C0161744|C0271431|C0271446|C0395849|C0477443|C04774 44|C0477445|C0477446|C0477447|C0477448|C0494087|C0694493|C1455742,otitis media,1,1,1,0,); (102,C0030319,fear ofpain,0,1,0,0,); (102,C0030319,fear of severe pain,0,1,0,0,); (102,C0030319,panic attack,1,0,0,0,); (103,C00305671|C1828079,parkinson disease,1,1,1,0,); (104,C0030809,pemphigus vulgaris,1,1,1,0,); (105,C0031090|C1314006,periodontal disease,1,0,1,0,); (106,C0035435,fibromyalgia,1,0,1,0,); (107,C0035436|C0264743|C03486841|C0 4095801|C0409651|C0451910|C3536892,rheumatic fever,1,1,0,0,); (108,C0035439|C0035440|C0155558|C0155711|C0348583|C0348584,rheumatic heart disease,1,1,0,0,); (109,C0035854|C0477490|C0477510,rosacea,1,0,1,0,); (110,C0036202|C003620 3|C0036204|C0036205|C0348540|C0348753|C0348826|C0451646|C0451751|C0451845|C04773 22|C0477329|C0477554|C0694513,sarcoidosis,1,1,0,0,); (111,C0036220|C0153464|C0153519|C 0153538|C0153560|C0153561|C0153562|C0153565|C0276535|C0348355|C0348364,kaposi sarcoma,1,0,1,1,GSE100684); (112,C0036285|C00366891|C0477446,scarlet fever,1,1,0,0,); (113,C003642|C0349082|C0451842|C0477589|C0494697,systemic sclerosis,1,1,1,1,GSE136731,
GSE136731); (114,C0036690|C0152486|C0152946|C0152964|C0152965|C0152966|C0152967|C 0269294|C0269936|C0275685|C0276029|C0276063|C0343453|C0348131|C0348133|C0348152| C0348970|C0349003|C0349004|C0349005|C0349006|C0349008|C0349009|C0392110|C045211 5|C0452197|C0452198|C0452199|C0477713|C0477736|C0477851|C0477919|C0477920|C04779 24|C0494048|C0494082|C0495304|C0495408|C0496128|C0496134|C0600327|C0868755|C0870 076|C3665339,sepsis,1,0,1,0,); (115,C0038013|C0348540|C0348715|C0409675|C0477606,ankyl osing spondylitis,1,1,1,0,); (116,C0038436|C0349255|C0494410|C0546983,insomnia,1,1,0,0,); (117,C0 038436|C0478140,suicidal ideation,1,1,0,0,); (118,C0040156|C0270222|C0477553|C0494276|C0494505,hyperthyroidism,1, 1,1,0,); (119,C0040592|C0153107|C0153108|C0348303,trachoma,1,0,1,0,); (120,C0040822|C027 0736|C0393519,essential tremor,1,1,0,0,); (121,C0041333|C0242172|C0348147|C0348149|C0348905|C0451784|C045178 5|C0452162|C0452163|C0494056,pelvic inflammatory disease,1,0,1,0,); (122,C0042024,stress urinary incontinence,1,1,0,0,); (123,C0042900|C0348510,vitiligo,1,1,1,0,); (124,C0085084,sporadic amyotrophic lateral sclerosis (sals),0,0,0,1,GSE122649); (124,C0085084,sporadic amyotrophic lateral sclerosis (sals) and frontotemporal dementia (ftd),0,0,0,1,GSE122649); (124,C0085084,als (amyotrophic lateral sclerosis) spectrum mnd (motor neurone diseases),0,0,0,1,GSE124439, GSE124439, GSE124439); (124,C0085084,amyotrophic lateral sclerosis,1,1,1,0,); (125,C0085096,intermittent claudication,1,0,0,0,); (125,C0085096,peripheral artery disease,0,1,0,0,); (126,C0085207|C0158915|C0477897|C1455664,diabetes (gestational),0,1,0,0,); (126,C0085207|C0158915|C0477897|C1455664,gestational diabetes,1,0,1,0,); (127,C0085215,premature ovarian failure,0,1,0,0,); (127,C0085215,primary ovarian failure,1,0,0,0,); (128,C00854371|C0477333|C0494441|C0694469,bacterial meningitis,0,1,0,0,); (128,C0085437|C0477333|C049444|C0694469,meningitis bacterial,1,0,0,0,); (129,C0085655|C04946971|C0546839,polymyositis,1,1,0,0,); (130,C01540091C 2937421,benign prostatic hyperplasia,1,1,0,0,); (130,C0154009|C2937421,benign prostatic hypertrophy,1,0,0,0,); (130,C0154009|C2937421,prostatic hypertrophy,1,0,0,0,); (131,C0155626|C0155668|C0155711|C0260469|C0340325|C0340331|C03 40442|C0348589|C0348591|C0348592|C0348593|C0348862|C0348863|C0348864|C0348865|C0 348866|C0348867|C0348876|C0348884|C0349074|C0476718|C0494577|C0494578|C0494579|C 0494580|C3665604,myocardial infarction,1,1,0,0,); (132,C0155709,atrial fibrillation,1,1,1,0,); (132,C0155709,atrial fibrillation and flutter,1,0,0,0,); (132,C0155709,atrial fibrillation/atrial flutter,0,1,0,0,); (132,C0155709,atrial flutter,1,0,0,0,); (133,C0156258|C0178288|C0451641,urolithiasis,1,1,1,0,); (134,C0156273|C0478 0341|C4317009,diverticulitis,1,1,0,0,); (135,C0178283,inflammatory bowel disease,1,1,1,0,); (136,C0178288|C0232867|C0269674|C0348859|C0451774|C0475548|C047776 2|C0477811|C0494218|C0495723|C3648968,proteinuria,1,1,0,0,); (137,C0238990,lower respiratory tract infection,1,0,1,0,); (138,C0263361,plaque psoriasis,1,0,0,0,); (138,C0263361,psoriasis vulgaris,0,1,0,0,); (139,C0263666|C0477588|C0494697|C3495559,dermatomyositis,1,1,0,0,); (14 0,C0265011|C0265012,abdominal aortic aneurysm,1,1,0,0,); (141,C02669291|C0392492,chronic periodontitis,1,1,0,0,); (142,C0270850,petit mal epilepsy,1,0,0,0,); (142,C0270850,juvenile myoclonic epilepsy,1,1,0,0,); (142,C0270850,atonic seizures,1,0,0,0,); (142,C0270850,childhood absence epilepsy,0,1,0,0,); (143,C0276609|C0276610|C0348199|C0400913|C0400918|C0451704|C04517 05|C0494092|C0494093,hepatitis b,1,1,1,0,); (144,C0338480|C1735856,aura,1,0,0,0,); (144,C0338480|C1735856,migraine without aura,1,1,0,0,); (145,C03388081|C03389701|C0476566|C0480203|C0494400,suicide,1,1,0,0,); (146, C0338960|C2267227,bulimia nervosa,1,1,0,0,); (147,C0338970|C1443045,borderline personality disorder,1,1,0,0,); (148,C0346724,basal cell carcinoma,1,1,0,0,); (149,C0348626,sinus rhythm,1,0,0,0,); (149,C0348626,long qt syndrome,1,0,0,0,); (149,C0348626,brugada syndrome,0,1,0,0,); (150,C0348645,berry aneurysm,1,0,0,0,); (150,C0348645,intracranial aneurysm,0,1,0,0,); (151,C0348694,silicosis,1,0,1,0,); (152,C0348703,idiopathic pulmonary fibrosis,1,1,1,0,); (152,C0348703,idiopathic pulmonary fibrosis (ipf),0,0,0,1,GSE79544, GSE73189, GSE138283, GSE52463); (153,C0348723|C0451753|C0477411|C0478155|C0494697|C1527336,sicca syndrome,0,0,1,0,); (153,C0348723|C0451753|C0477411|C0478155|C0494697|C1527336,syndro me sicca,1,0,0,0,); (154,C0349215,bipolar ii disorder,1,1,0,0,); (155,C0349258,sexual dysfunction (female),0,1,0,0,); (155,C0349258,erectile dysfunction,1,1,0,0,); (156,C03493491|C0579986,attention deficit disorder,1,0,0,0,); (156,C03493491|C0579986,attention deficit hyperactivity disorder,0,1,1,0,); (156,C03493491|C0579986,attention deficit/hyperactivity disorder,1,0,0,0,); (157,C0400914|C0524910,hepatitis c,1,0,1,0,); (158,C0404572,anovulation,1,0,1,0,); (159,C0433856|C0452047,traumatic brain injury, 1,0,1,0,); (160,C04517071|C0728889,cholestasis,1,0,1,0,); (161,C0451708|C0451710|C0451 711|C0451712|C0494794,chronic liver disease,1,0,1,0,); (162,C0476479,anger,1,1,0,0,); (163,C0476643|C04766841|C0481423|C0556008|C0694464,drug abuse,1,1,0,0,); (164,C0477362,akathisia,1,0,0,0,); (164,C0477362,restless legs syndrome,1,1,0,0,); (164,C0477362,stiff person syndrome,1,0,0,0,); (165,C0477491,chronic urticaria,0,0,1,0,); (165,C0477491,urticaria chronic,1,0,0,0,); (166,C0494026,Clostridium difficile infection,1,0,1,0,); (167,C0494242,clotting,1,0,0,0,); (167,C0494242,factor vii,0,1,0,0,); (167,C0494242,fibrinogen,0,1,0,0,); (168,C0494372,sleep duration,0,1,0,0,); (168,C0494372,delirium tremens,1,0,0,0,); (169,C0494650|C0494660,allergic asthma,1,0,1,0,); (170,C0494679,interstitial lung disease,1,1,0,0,); (171,C0494697|C0494887|C0495047|C3495801,granulomatosis with polyangiitis,0,0,1,0,); (171,C0494697|C0494887|C0495047|C3495801,wegener granulomatosis,1,1,0,0,); (172,C0494783,hepatic encephalopathy,1,0,1,0,); (173,C0494831,atopic eczema,0,0,1,0,); (173,C0494831,eczema infantile,1,0,0,0,); (173,C0494831,infantile eczema,0,0,1,0,); (174,C0495723,albuminuria,1,1,0,0,); (175,C0496594,alcoholism,1,0,1,0,); (176, C0496856,thyroid carcinoma,1,0,1,0,); (177,C0751362|C1305395,narcolepsy,1,1,0,0,); (178,C0995154,chronic back pain,0,1,0,0,); (178,C0995154,eye pain,1,0,0,0,); (179,C1265999,bowen disease,1,0,0,0,); (179,C1265999,cervical cancer,0,1,1,0,); (180,C1305395,daytime sleepiness,0,1,0,0,); (180,C1305395,excessive daytime sleepiness,1,1,0,0,); (181,C1443045,obsessive-compulsive symptoms,0,1,0,0,); (181,C1443045,obsessive-compulsive disorder,1,1,0,0,); (182,C1735856,acute migraine,1,0,0,0,); (182,C1735856,basilar migraine,1,0,0,0,); (182,C1735856,classical migraine,1,0,0,0,); (182,C1735856,hemiplegic migraine,1,0,0,0,); (182,C1735856,migraine with aura,0,1,0,0,); (183,C2239176,hepatocellular carcinoma,1,1,0,0,); (184,adenocarcinoma,adenocarcinoma,1,1,0,0,); (185,age-related macular degeneration,age-related macular degeneration,1,1,0,0,); (186,barrett oesophagus,barrett oesophagus,1,0,1,0,); (187,bipolar i disorder,bipolar i disorder,1,1,0,0,); (188,breast cancer,breast cancer,1,1,1,1,GSE124030); (188,breast cancer,breast cancer (dcis; ductal carcinoma in situ),0,0,0,1,GSE117970); (188,breast cancer,breast cancer (invasive breast cancer),0,0,0,1,GSE117970, GSE117970); (188,breast cancer,breast cancer (luminal b type),0,0,0,1,GSE110114); (189,candidemia,candidemia,1,1,0,0,); (190,colorectal cancer,colorectal cancer,1,1,1,0,); (190,colorectal cancer,colorectal tumor (moderately differentiated),0,0,0,1,GSE109203); (190,colorectal cancer,colorectal tumor (poorly differentiated),0,0,0,1,GSE109203); (190,colorectal cancer,colorectal tumor (well differentiated),0,0,0,1,GSE109203); (190,colorectal cancer,metastatic colorectal cancer to the liver (stage iv),0,0,0,1,GSE50760); (191,dandruff,dandruff,1,0,1,0,); (192,dementia with lewy bodies,dementia with lewy bodies,1,1,0,0,); (193,diabetic nephropathy,diabetic nephropathy,1,0,1,0,); (193,diabetic nephropathy,early diabetic nephropathy (dn),0,0,0,1,GSE142025); (194,endometrial cancer,endometrial cancer (upsc; uterine papillary serous carcinoma),0,0,0,1,GSE117970, GSE117970); (194,endometrial cancer,endometrial cancer (endometrioid),0,0,0,1,GSE117970, GSE117970); (194,endometrial cancer,endometrial tumor,0,0,0,1,GSE56087, GSE146889, GSE146889, GSE146889, GSE146889); (194,endometrial cancer,endometrial cancer (carcinoma),0,0,0,1,GSE117970); (194,endometrial cancer,endometrial cancer,1,1,1,0,); (194,endometrial cancer,endometrial cancer (carcinosarcoma),0,0,0,1,GSE117970); (195,ewing sarcoma,ewing sarcoma,1,1,0,0,); (196,feeling guilty,feeling guilty,1,1,0,0,); (197,glioblastoma,glioblastoma,1,1,0,0,); (198,glioma,glioma,1,1,0,0,); (199,hepar in-induced thrombocytopenia,heparin-induced thrombocytopenia,1,1,0,0,); (200,juvenile idiopathic arthritis,juvenile idiopathic arthritis,1,0,1,0,); (201,mean arterial pressure,mean arterial pressure,1,1,0,0,); (202,microalbuminuria,microalbuminuria,1,1,0,0,); (203,neuroblastoma,neurob lastoma,1,1,0,0,); (204,nicotine dependence,nicotine dependence,1,1,0,0,); (205,non-small cell lung cancer,non-small cell lung cancer,1,1,0,0,); (206,osteosarcoma,osteosarcoma,1,1,0,0,); (207,ovarian cancer,ovarian cancer,1,1,0,0,); (208,post-traumatic stress disorder,post-traumatic stress disorder,1,1,0,0,); (209,prostate cancer,prostate cancer,1,1,1,0,); (210,reflux esophagitis,reflux esophagitis,1,0,1,0,); (211,renal cell carcinoma,renal cell carcinoma,1,1,0,0,); (212,sjogren syndrome,sjogren syndrome,1,0,1,0,); (213,stevens-johnson syndrome,stevens-johnson syndrome,1,0,1,0,)₀
   <Listing 3: Novel marker>

Data are listed in the order (Disease ID for figure, UMLS ID, Tissuegene, count). (1,C0001144|C0001145|C0022548|C0029485|C0152249|C0263437|C0263442|C0494863|C0702 166,Whole_blood_AKIRIN2,2911); (1,C00011441|C0001145|C00225481|C0029485|C0152249|C0 2634371|C0263442|C0494863|C0702166,Monocyte PER2,2255); (1,C00011441|C0001145|C0022 548|C0029485|C0152249|C0263437|C0263442|C0494863|C0702166,Colon_RAMP2,2035); (1,C 0001144|C0001145|C0022548|C0029485|C0152249|C0263437|C0263442|C0494863|C0702166, Unknown_CD86,1905); (1,C0001144|C0001145|C0022548|C0029485|C0152249|C0263437|C02 63442|C0494863|C0702166,Monocyte_RILP,1893); (1,C0001144|C0001145|C0022548|C002948 5|C0152249|C0263437|C0263442|C0494863|C0702166,Monocyte_SNX9,1875); (1,C0001144|C 0001145|C0022548|C0029485|C0152249|C02634371|C0263442|C0494863|C0702166,Endometri al tissue_TVP23CP2,1868); (1,C0001144|C0001145|C0022548|C0029485|C0152249|C0263437|C0 263442|C0494863|C0702166,Monocyte_EIF3G,1705); (1,C0001144|C0001145|C0022548|C0029 485|C01522491|C02634371|C0263442|C0494863|C0702166,Endometrial tissue_SMC2,1699); (1,C0001144|C0001145|C0022548|C0029485|C0152249|C0263437|C02634 42|C0494863|C0702166,Temporal_cortex_TRIM37,1687); (1,C0001144|C0001145|C0022548|C 0029485|C0152249|C0263437|C0263442|C0494863|C0702166,Colorectum_VPS37B,1656); (1,C 0001144|C0001145|C0022548|C0029485|C0152249|C0263437|C0263442|C0494863|C0702166, Brain white matter_STON1-GTF2A1L,1647); (1,C00001144|C0001145|C0022548|C0029485|C0152249|C0263437|C0263442| C0494863|C0702166,Endometrial tissue_TMEM159,1635); (1,C0001144|C0001145|C0022548|C0029485|C0152249|C0263437|C0 263442|C0494863|C0702166,Endometrial tissue_GLB1,1550); (1,C0001144|C0001145|C0022548|C0029485|C0152249|C0263437|C02634 42|C0494863|C0702166,Monocyte_PDHX,1494); (1,C00011441|C0001145|C0022548|C0029485|C0152249|C0263437|C0263442|C0494863|C0702166,Bladder_THEM6,1490); (1,C0001144|C00 01145|C0022548|C0029485|C0152249|C0263437|C0263442|C0494863|C0702166,Temporal_co rtex_MYADML2,1475); (1,C0001144|C0001145|C0022548|C0029485|C0152249|C0263437|C02 63442|C0494863|C0702166,Brain white matter_CLEC12A,1473); (1,C0001144|C0001145|C0022548|C0029485|C0152249|C0263437|C0 263442|C0494863|C0702166,Macrophage_MEPE,1424); (1,C0001144|C0001145|C0022548|C00 29485|C0152249|C0263437|C0263442|C0494863|C0702166,Endometrial tissue_PODN,1416); (2,C0001339|C0153094|C0341465|C0341470|C0348760|C0348762,Whole_blood_AKIRIN2,2911); (2,C0001339|C0153094|C0341465|C0341470|C03487601|C0348762,Mon ocyte_PER2,2255); (2,C0001339|C0153094|C0341465|C0341470|C0348760|C0348762,Colon_R AMP2,2035); (2,C0001339|C0153094|C0341465|C0341470|C0348760|C0348762,Unknown_CD 86,1905); (2,C0001339|C0153094|C0341465|C0341470|C0348760|C0348762,Monocyte_RILP,1 893); (2,C0001339|C0153094|C0341465|C0341470|C0348760|C0348762,Monocyte_SNX9,1875); (2,C0001339|C0153094|C0341465|C0341470|C0348760|C0348762,Endometrial tissue_TVP23CP2,1868); (2,C0001339|C01530941|C0341465|C0341470|C03487601|C0348762,Mo nocyte_EIF3G,1705); (2,C0001339|C0153094|C0341465|C0341470|C0348760|C0348762,Endom etrial tissue_SMC2,1699); (2,C0001339|C0153094|C0341465|C0341470|C0348760|C0348762,Tempor al_cortex_TRIM37,1687); (2,C0001339|C0153094|C0341465|C0341470|C0348760|C0348762,C olorectum_VPS37B,1656); (2,C0001339|C0153094|C0341465|C0341470|C0348760|C0348762,B rain white matter_STON1-GTF2A1L,1647); (2,C0001339|C0153094|C0341465|C0341470|C0348760|C0348762,Endometri al tissue_TMEM159,1635); (2,C0001339|C0153094|C0341465|C0341470|C0348760|C0348762,En dometrial tissue_GLB1,1550); (2,C0001339|C0153094|C0341465|C0341470|C0348760|C0348762,Monocy te_PDHX,1494); (2,C0001339|C0153094|C0341465|C0341470|C0348760|C0348762,Bladder_T HEM6,1490); (2,C0001339|C0153094|C0341465|C0341470|C0348760|C0348762,Temporal_cort ex_MYADML2,1475); (2,C0001339|C0153094|C0341465|C0341470|C0348760|C0348762,Brain white matter_CLEC12A,1473); (2,C0001339|C0153094|C0341465|C0341470|C0348760|C0348762,Ma crophage_MEPE,1424); (2,C0001339|C0153094|C0341465|C0341470|C0348760|C0348762,End ometrial tissue_PODN,1416); (9,C0002171|C0477504,Whole_blood_AKIRIN2,2911); (9,C0002171|C047 7504,Monocyte_PER2,2255); (9,C0002171|C0477504,Colon_RAMP2,2035); (9,C0002171|C047 7504,Unknown_CD86,1905); (9,C000217|C0477504,Monocyte_RILP,1893); (9,C0002171|C04 77504,Monocyte_SNX9,1875); (9,C000217|C0477504,Endometrial tissue_TVP23CP2,1868); (9,C000217|C0477504,Monocyte_EIF3G,1705); (9,C000217|C04775 04,Endometrial tissue_SMC2,1699); (9,C0002171|C0477504,Temporal_cortex_TRIM37,1687); (9,C0002171|C0 477504,Colorectum_VPS37B,1656); (9,C0002171|C0477504,Brain white matter_STON1-GTF2A1L,1647); (9,C0002171|C0477504,Endometrial tissue_TMEM159,1635); (9,C000217|C0477504,Endometrial tissue_GLB1,1550); (9,C0002171|C0477504,Monocyte_PDHX,1494); (9,C0002171|C0477504,B1 adder_THEM6,1490); (9,C0002171|C0477504,Temporal_cortex_MYADML2,1475); (9,C000217 1|C0477504,Brain white matter_CLEC12A,1473); (9,C000217|C0477504,Macrophage_MEPE,1424); (9,C000217|C047 7504,Endometrial tissue_PODN,1416); (10,C0003123|C0003125|C0338959|C0494440|C2267227,Whole_blood_A KIRIN2,2911); (10,C0003123|C0003125|C0338959|C0494440|C2267227,Monocyte_PER2,2255); (10,C0003123|C0003125|C0338959|C04944401|C2267227,Colon_RAMP2,2035); (10,C0003123|C0003125|C0338959|C0494440|C2267227,Unknown_CD86,1905); (10,C0003123|C0003125|C0 338959|C0494440|C2267227,Monocyte_RILP,1893); (10,C0003123|C0003125|C0338959|C0494 440|C2267227,Monocyte_SNX9,1875); (10,C0003123|C0003125|C0338959|C04944401|C226722 7,Endometrial tissue_TVP23CP2,1868); (10,C0003123|C0003125|C0338959|C0494440|C2267227,Monocyte_E IF3G,1705); (10,C0003123|C0003125|C0338959|C0494440|C2267227,Endometrial tissue_SMC2,1699); (10,C0003123|C0003125|C0338959|C0494440|C2267227,Temporal_cortex_TRIM37,1687); (10,C00003123|C0003125|C0338959|C0494440|C2267227,Colorectum_VPS37 B,1656); (10,C0003123|C0003125|C03389591|C0494440|C2267227,Brain white matter_STON1-GTF2A1L,1647); (10,C0003123|C0003125|C0338959|C0494440|C2267227,Endometrial tissue_TMEM159,1635); (10,C0003123|C0003125|C0338959|C0494440|C2267227,Endometrial tissue_GLB1,1550); (10,C0003123|C0003125|C0338959|C0494440|C2267227,Monocyte_PDHX ,1494); (10,C0003123|C0003125|C0338959|C0494440|C2267227,Bladder_THEM6,1490); (10,C0 003123|C0003125|C0338959|C0494440|C2267227,Temporal_cortex_MYADML2,1475); (10,C0 003123|C00003125|C03389591|C0494440|C2267227,Brain white matter_CLEC12A,1473); (10,C0003123|C0003125|C0338959|C0494440|C2267227,Macrophage_MEPE,1424); (10,C0003123|C0003125|C0338959|C0494440|C2267227,Endometrial tissue_PODN,1416); (13,C0003615|C0031154|C0085693|C0152505|C0156095,Whole_blood_A KIRIN2,2911); (13,C0003615|C0031154|C0085693|C0152505|C0156095,Monocyte_PER2,2255); (13,C0003615|C0031154|C0085693|C0152505|C0156095,Colon_RAMP2,2035); (13,C0003615|C0031154|C0085693|C0152505|C0156095,Unknown_CD86,1905); (13,C0003615|C0031154|C0 085693|C0152505|C0156095,Monocyte_RILP,1893); (13,C0003615|C0031154|C0085693|C0152 505|C0156095,Monocyte_SNX9,1875); (13,C0003615|C0031154|C0085693|C0152505|C015609 5,Endometrial tissue_TVP23CP2,1868); (13,C0003615|C0031154|C0085693|C0152505|C0156095,Monocyte_E IF3G,1705); (13,C0003615|C0031154|C0085693|C0152505|C0156095,Endometrial tissue_SMC2,1699); (13,C0003615|C0031154|C0085693|C0152505|C0156095,Temporal_cortex_TRIM37,1687); (13,C0003615|C0031154|C0085693|C0152505|C0156095,Colorectum_VPS37 B,1656); (13,C0003615|C00311541|C0085693|C0152505|C0156095,Brain white matter_STON1-GTF2A1L,1647); (13,C0003615|C0031154|C0085693|C0152505|C0156095,Endometrial tissue_TMEM159,1635); (13,C0003615|C0031154|C0085693|C0152505|C0156095,Endometrial tissue_GLB1,1550); (13,C0003615|C0031154|C0085693|C0152505|C0156095,Monocyte_PDHX ,1494); (13,C0003615|C0031154|C0085693|C0152505|C0156095,Bladder_THEM6,1490); (13,C0 003615|C0031154|C0085693|C0152505|C0156095,Temporal_cortex_MYADML2,1475); (13,C0 003615|C00311541|C0085693|C0152505|C0156095,Brain white matter_CLEC12A,1473); (13,C0003615|C0031154|C0085693|C0152505|C0156095,Macrophage_MEPE,1424); (13,C0003615|C0031154|C0085693|C0152505|C0156095,Endometrial tissue_PODN,1416); (16,C0003873|C0015773|C0026848|C0029660|C0038013|C0409651|C0409 652|C0477411|C0477541|C0477542|C0494896|C0494897|C0694497|C3714757,Whole_blood_AKIRIN2,2911); (16,C0003873|C0015773|C0026848|C0029660|C0038013|C0409651|C0409652|C0477411|C0477541|C0477542|C0494896|C0494897|C0694497|C3714757,Monocyte_PER2,22 55); (16,C0003873|C0015773|C0026848|C00296601|C0038013|C0409651|C0409652|C0477411|C 0477541|C0477542|C0494896|C0494897|C0694497|C3714757,Colon_RAMP2,2035); (16,C0003 873|C0015773|C0026848|C0029660|C0038013|C0409651|C0409652|C0477411|C0477541|C047 7542|C0494896|C0494897|C0694497|C3714757,Unknown_CD86,1905); (16,C0003873|C00157 731|C0026848|C0029660|C0038013|C0409651|C0409652|C0477411|C0477541|C0477542|C0494 896|C0494897|C0694497|C3714757,Monocyte_RILP,1893); (16,C0003873|C0015773|C0026848|C00029660|C0038013|C0409651|C0409652|C0477411|C0477541|C0477542|C0494896|C049489 7|C0694497|C3714757,Monocyte_SNX9,1875); (16,C0003873|C0015773|C00268481|C0029660|C00038013|C0409651|C0409652|C0477411|C0477541|C0477542|C0494896|C0494897|C069449 7|C3714757,Endometrial tissue_TVP23CP2,1868); (16,C0003873|C0015773|C0026848|C0029660|C0038013|C040965|C 0409652|C0477411|C0477541|C0477542|C0494896|C0494897|C0694497|C3714757,Monocyte_EIF3G,1705); (16,C0003873|C0015773|C0026848|C0029660|C0038013|C0409651|C0409652|C0 477411|C0477541|C0477542|C0494896|C0494897|C0694497|C3714757,Endometrial tissue_SMC2,1699); (16,C0003873|C0015773|C0026848|C0029660|C0038013|C0409651|C0409 652|C0477411|C0477541|C0477542|C0494896|C0494897|C0694497|C3714757,Temporal_corte x_TRIM37,1687); (16,C0003873|C0015773|C0026848|C0029660|C0038013|C0409651|C040965 2|C0477411| C0477541|C0477542|C0494896|C0494897|C0694497|C3714757,Colorectum_VPS3 7B,1656); (16,C0003873|C0015773|C0026848|C0029660|C0038013|C0409651|C0409652|C0477 411|C0477541|C0477542|C0494896|C04948971|C0694497|C3714757,Brain white matter_STON1-GTF2A1L,1647); (16,C0003873|C0015773|C0026848|C0029660|C0038013|C0409651|C040965 2|C477542|C0494896|C0494897|C0694497|C3714757,Endometrial tissue_TMEM159,1635); (16,C0003873|C0015773|C0026848|C0029660|C0038013|C0409651|C 0409652|C0477411|C0477541|C0477542|C0494896|C0494897|C0694497|C3714757,Endometri al tissue_GLB1,1550); (16,C0003873|C0015773|C0026848|C0029660|C0038013|C0409651|C0409 652|C0477411|C0477541|C0477542|C0494896|C0494897|C0694497|C3714757,Monocyte_PDH X,1494); (16,C0003873|C0015773|C0026848|C0029660|C0038013|C0409651|C0409652|C04774 11|C0477541|C0477542|C0494896|C0494897|C0694497|C3714757,Bladder_THEM6,1490); (16, C0003873|C0015773|C0026848|C0029660|C0038013|C0409651|C0409652|C0477411|C047754 1| C0477542| C0494896| C04948971|C0694497|C3714757,Temporal_cortex_MYADML2,1475); (1 6,C0003873|C0015773|C0026848|C00296601|C0038013|C0409651|C0409652| C0477411| C04775 41|C0477542|C0494896|C04948971|C0694497|C3714757,Brain white matter_CLEC12A,1473); (16,C0003873|C0015773|C0026848|C0029660|C0038013|C0409651|C 0409652|C0477411|C0477541|C0477542|C0494896|C0494897|C0694497|C3714757,Macrophag e_MEPE,1424); (16,C0003873|C0015773|C0026848|C0029660|C0038013|C0409651|C0409652|C0477411|C0477541|C0477542|C0494896|C0494897|C0694497|C3714757,Endometrial tissue_PODN,1416); (17,C0003962|C0036528|C0152717|C0156180|C0477842|C0495216|C0495 676|C0495700,Whole_blood_AKIRIN2,2911); (17,C0003962|C0036528|C0152717|C0156180|C 0477842|C0495216|C0495676|C0495700,Monocyte_PER2,2255); (17,C0003962|C0036528|C01 527171|C01561801|C0477842| C0495216| C0495676| C0495700,Colon_RAMP2,2035); (17,C000396 2|C0036528|C0152717|C0156180|C0477842|C0495216|C0495676|C0495700,Unknown_CD86,1 905); (17,C0003962|C0036528|C0152717|C0156180|C0477842|C0495216|C0495676|C0495700, Monocyte_RILP,1893); (17,C0003962|C0036528|C0152717|C0156180|C0477842|C0495216|C0 495676|C0495700,Monocyte_SNX9,1875); (17,C0003962|C0036528|C0152717|C0156180|C047 7842|C0495216|C0495676|C0495700,Endometrial tissue_TVP23CP2,1868); (17,C0003962|C0036528|C0152717|C0156180|C0477842|C0495216|C 0495676|C0495700,Monocyte_EIF3G,1705); (17,C0003962|C0036528|C0152717|C0156180|C04 77842|C0495216|C0495676|C0495700,Endometrial tissue_SMC2,1699); (17,C0003962|C0036528|C0152717|C0156180|C0477842|C0495216|C0495 676|C0495700,Temporal_cortex_TRIM37,1687); (17,C0003962|C0036528|C0152717|C0156180|C0477842|C0495216|C0495676|C0495700,Colorectum_VPS37B,1656); (17,C0003962|C003652 8|C0152717|C01561801|C0477842|C0495216|C0495676|C0495700,Brain white matter_STON1-GTF2A1L,1647); (17,C0003962|C0036528|C0152717|C0156180|C0477842|C0495216|C049567 6|C0495700,Endometrial tissue_TMEM159,1635); (17,C00003962|C0036528|C0152717|C0156180|C0477842|C0495216|C 0495676|C0495700,Endometrial tissue_GLB1,1550); (17,C0003962|C0036528|C0152717|C0156180|C0477842|C0495216|C0495 676|C0495700,Monocyte_PDHX,1494); (17,C0003962|C0036528|C0152717|C01561801|C047784 2|C0495216|C0495676|C0495700,Bladder_THEM6,1490); (17,C0003962|C0036528|C0152717| C01561801|C0477842|C0495216|C0495676|C0495700,Temporal_cortex_MYADML2,1475); (17, C0003962|C0036528|C0152717|C0156180|C0477842|C0495216|C0495676|C0495700,Brain white matter_CLEC12A,1473); (17,C0003962|C0036528|C0152717|C0156180|C0477842|C0495216|C 0495676|C0495700,Macrophage_MEPE,1424); (17,C00003962|C0036528|C0152717|C0156180|C 0477842|C0495216|C0495676|C0495700,Endometrial tissue_PODN,1416); (19,C00004153|C0007775|C0010054|C0017327|C0155733|C0155734|C0348 648|C0494764|C0495064|C0495663|C3495604,Whole_blood_AKIRIN2,2911); (19,C0004153|C 0007775|C0010054|C0017327|C0155733|C0155734|C0348648|C0494764|C0495064|C0495663| C3495604,Monocyte_PER2,2255); (19,C0004153|C0007775|C00100541|C0017327|C0155733|C0 155734|C0348648|C0494764|C0495064|C0495663|C3495604,Colon_RAMP2,2035); (19,C00041 53|C0007775|C0010054|C0017327|C0155733|C0155734|C03486481|C0494764|C04950641|C0495 663|C3495604,Unknown_CD86,1905); (19,C0004153|C0007775|C0010054|C0017327|C015573 3|C0155734|C0348648|C0494764|C0495064|C0495663|C3495604,Monocyte_RILP,1893); (19,C 0004153|C0007775|C0010054|C0017327|C0155733|C0155734|C0348648|C0494764|C0495064| C0495663|C3495604,Monocyte_SNX9,1875); (19,C0004153|C0007775|C0010054|C0017327|C0 155733|C0155734|C0348648|C0494764|C0495064|C0495663|C3495604,Endometrial tissue_TVP23CP2,1868); (19,C0004153|C0007775|C0010054|C0017327|C0155733|C0155734|C 0348648|C0494764|C0495064|C0495663|C3495604,Monocyte_EIF3G,1705); (19,C0004153|C00 07775|C0010054|C0017327|C0155733|C0155734|C0348648|C0494764|C0495064|C0495663|C3 495604,Endometrial tissue_SMC2,1699); (19,C0004153|C0007775|C0010054|C0017327|C0155733|C0155734|C0348 648|C0494764|C0495064|C0495663|C3495604,Temporal_cortex_TRIM37,1687); (19,C0004153| C0007775|C0010054|C0017327|C0155733|C0155734|C0348648|C0494764|C0495064|C049566 3|C3495604,Colorectum_VPS37B,1656); (19,C0004153|C0007775|C00100541|C0017327|C01557 33|C01557341|C0348648|C04947641|C04950641|C0495663|C3495604,Brain white matter_STON1-GTF2A1L,1647); (19,C0004153|C0007775|C00100541|C0017327|C0155733|C0155734|C034864 8|C04947641|C04950641|C0495663|C3495604,Endometrial tissue_TMEM159,1635); (19,C0004153|C0007775|C0010054|C0017327|C0155733|C0155734|C 0348648|C0494764|C0495064|C0495663|C3495604,Endometrial tissue_GLB1,1550); (19,C0004153|C0007775|C0010054|C0017327|C0155733|C0155734|C0348 648|C04947641|C04950641|C0495663|C3495604,Monocyte PDHX,1494); (19,C0004153|C000777 5|C0010054|C0017327|C0155733|C0155734|C0348648|C0494764|C0495064|C0495663|C34956 04,Bladder_THEM6,1490); (19,C0004153|C0007775|C0010054|C0017327|C0155733|C0155734| C03486481|C04947641|C04950641|C0495663|C3495604,Temporal_cortex_MYADML2,1475); (19, C0004153|C0007775|C0010054|C0017327|C0155733|C0155734|C0348648|C0494764|C049506 41|C0495663|C3495604,Brain white matter_CLEC12A,1473); (19,C0004153|C0007775|C0010054|C0017327|C0155733|C0155734|C 0348648|C0494764|C0495064|C0495663|C3495604,Macrophage_MEPE,1424); (19,C0004153|C 0007775|C0010054|C0017327|C0155733|C0155734|C0348648|C0494764|C0495064|C0495663| C3495604,Endometrial tissue_PODN,1416); (21,C0004626|C0032285|C00323001|C0032302|C00323081|C00323101|C0034 063|C0152413|C0152487|C0152502|C0155858|C0155860|C0155862|C0158935|C0158944|C020 6062|C0260334|C0264522|C0276026|C0276089|C0276156|C0276333|C0339951|C0339959|C03 39961|C0339969|C0339971|C0343320|C0343323|C0343324|C0343666|C0348152|C0348161|C0 348193|C0348677|C0348678|C0348679|C0348680|C0348681|C0348684|C0348685|C0348703|C 0348801|C0348804|C0348806|C0348809|C0348814|C0348968|C0348970|C0349359|C0349468| C0375326|C0477908|C0477909|C0494034|C0494059|C0494086|C0494137|C0494638|C049463 9|C0494640|C0494643|C0494644|C0494675|C0494678|C0494683|C0495389|C0495390|C05190 30|C0694504|C0869210,Whole_blood_AKIRIN2,2911); (21,C0004626|C0032285|C0032300|C0 032302|C0032308|C0032310|C0034063|C0152413|C0152487|C0152502|C0155858|C0155860|C 0155862|C0158935|C0158944|C0206062|C0260334|C0264522|C0276026|C0276089|C0276156| C0276333|C0339951|C0339959|C0339961|C0339969|C0339971|C0343320|C0343323|C034332 4|C0343666|C0348152|C0348161|C0348193|C0348677|C0348678|C0348679|C0348680|C03486 81|C0348684|C0348685|C0348703|C0348801|C0348804|C0348806|C0348809|C0348814|C0348 968|C0348970|C0349359|C0349468|C0375326|C0477908|C0477909|C0494034|C0494059|C049 4086|C0494137|C0494638|C0494639|C0494640|C0494643|C0494644|C0494675|C0494678|C04 94683|C04953891|C04953901|C05190301|C06945041|C0869210,Monocyte PER2,2255); (21,C0004 626|10032285|C0032300|C0032302|C0032308|C0032310|C0034063|C0152413|C0152487|C015 2502|C0155858|C0155860|C0155862|C0158935|C0158944|C0206062|C0260334|C0264522|C02 76026|C0276089|C0276156|C0276333|C0339951|C0339959|C0339961|C0339969|C0339971|C0 343320|C0343323|C0343324|C0343666|C0348152|C0348161|C0348193|C0348677|C0348678|C 0348679|C0348680|C0348681|C0348684|C0348685|C0348703|C0348801|C0348804|C0348806| C0348809|C0348814|C0348968|C0348970|C0349359|C0349468|C0375326|C0477908|C047790 9|C0494034|C0494059|C0494086|C0494137|C0494638|C0494639|C0494640|C0494643|C04946 44|C0494675|C0494678|C0494683|C0495389|C0495390|C0519030|C0694504|C0869210,Colon RAMP2,2035); (21,C0004626|C0032285|C0032300|C0032302|C0032308|C0032310|C0034063| C0152413|C0152487|C0152502|C0155858|C0155860|C0155862|C0158935|C0158944|C020606 2|C0260334|C0264522|C0276026|C0276089|C0276156|C0276333|C0339951|C0339959|C03399 61|C0339969|C0339971|C0343320|C0343323|C0343324|C0343666|C0348152|C0348161|C0348 193|C0348677|C0348678|C0348679|C0348680|C0348681|C0348684|C0348685|C0348703|C034 8801|C0348804|C0348806|C0348809|C0348814|C0348968|C0348970|C0349359|C0349468|C03 75326|C0477908|C0477909|C0494034|C0494059|C0494086|C0494137|C0494638|C0494639|C0 4946401|C0494643|C0494644|C0494675|C0494678|C04946831|C0495389|C0495390|C0519030|C 0694504|C0869210,Unknown_CD86,1905); (21,C0004626|C0032285|C0032300|C0032302|C00 32308|C0032310|C0034063|C0152413|C0152487|C0152502|C0155858|C0155860|C0155862|C0 158935|C0158944|C0206062|C0260334|C0264522|C0276026|C0276089|C0276156|C0276333|C 0339951|C0339959|C0339961|C0339969|C0339971|C0343320|C0343323|C0343324|C0343666| C0348152|C0348161|C0348193|C0348677|C0348678|C0348679|C0348680|C0348681|C034868 4|C0348685|C0348703|C0348801|C0348804|C0348806|C0348809|C0348814|C0348968|C03489 70|C0349359|C0349468|C0375326|C0477908|C0477909|C0494034|C0494059|C0494086|C0494 137|C0494638|C0494639|C0494640|C0494643|C0494644|C0494675|C0494678|C0494683|C049 53891|C04953901|C05190301|C06945041|C0869210,Monocyte_RILP,1893); (21,C0004626|C003228 5|C0032300|C0032302|C0032308|C0032310|C0034063|C0152413|C0152487|C0152502|C01558 58|C0155860|C0155862|C0158935|C0158944|C0206062|C0260334|C0264522|C0276026|C0276 089|C0276156|C0276333|C0339951|C0339959|C0339961|C0339969|C0339971|C0343320|C034 3323|C0343324|C0343666|C0348152|C0348161|C0348193|C0348677|C0348678|C0348679|C03 48680|C0348681|C0348684|C0348685|C0348703|C0348801|C0348804|C0348806|C0348809|C0 348814|C0348968|C03489708|C0349359|C0349468|C0375326|C0477908|C0477909|C0494034|C 0494059|C0494086|C0494137|C0494638|C0494639|C0494640|C0494643|C0494644|C0494675|C0494678|C0494683|C0495389|C0495390|C0519030|C0694504|C0869210,Monocyte_SNX9,18 75); (21,C0004626|C0032285|C0032300|C0032302|C0032308|C0032310|C0034063|C0152413|C 0152487|C0152502|C0155858|C0155860|C0155862|C0158935|C0158944|C0206062|C0260334| C0264522|C0276026|C02760891|C0276156|C0276333|C0339951|C0339959|C0339961|C033996 9|C0339971|C0343320|C0343323|C0343324|C0343666|C0348152|C0348161|C0348193|C03486 77|C0348678|C0348679|C0348680|C0348681|C0348684|C0348685|C0348703|C0348801|C0348 804|C0348806|C0348809|C0348814|C0348968|C0348970|C0349359|C0349468|C0375326|C047 7908|C0477909|C091|C3|C0494086|C04941371|C94638|C04946 940|C04 946431|C0494644|C0494675|C0494678|C0494683|C0495389|C0495390|C0519030|C0694504|C0 869210,Endometrial tissue_TVP23CP2,1868); (21,C0004626|C0032285|C0032300|C0032302|C0032308|C0032310|C 0034063|C0152413|C0152487|C0152502|C0155858|C0155860|C0155862|C0158935|C0158944| C0206062|C0260334|C0264522|C0276026|C0276089|C0276156|C0276333|C0339951|C033995 9|C0339961|C0339969|C0339971|C0343320|C0343323|C0343324|C0343666|C0348152|C03481 61|C0348193|C0348677|C0348678|C0348 46801|C034868|C03486841|C0348685|C0348 703|C0348801|C0348804|C0348806|C0348809|C0348814|C0348968|C0348970|C0349359|C034 9468|C0375326|C0477908|C0477909|C0|C0494086|C0494137|C0494638|C04 94639|C0494640|C0494643|C0494644|C0494675|C0494678|C0494683|C0495389|C0495390|C0 519030|C0694504|C0869210,Monocyte_EIF3G,1705); (21,C0004626|C0032285|C00323001|C003 2302|C0032308|C0032310|C0034063|C0152413|C0152487|C0152502|C0155858|C0155860|C01 55862|C0158935|C0158944|C0206062|C0260334|C0264522|C0276026|C0276089|C0276156|C0 276333|C0339951|C0339959|C0339961|C0339969|C0339971|C0343320|C0343323|C0343324|C 0343666|C0348152|C0348161|C0348193|C0348677|C0348678|C0348679|C0348680|C0348681| C0348684|C0348685|C0348703|C0348801|C0348804|C0348806|C0348809|C0348814|C034896 8|C0348970|C0349359|C0349468|C0375326|C0477908|C0477909|C0494034|C0494059|C04940 86|C0494137|C0494638|C0494639|C0494640|C0494643|C0494644|C0494675|C0494678|C0494 683|C04953891|C04953901|C05190301|C06945041|C0869210,Endometrial tissue_SMC2,1699); (21,C0004626|C0032285|C0032300|C0032302|C0032308|C0032310|C0034 063|C0152413|C0152487|C0152502|C0155858|C0155860|C0155862|C0158935|C0158944|C020 6062|C0260334|C0264522|C0276333CC0339951|C0339959|C03 348193|C0348677|C0348 3679|C0348680|C0348681|C0348684|C0348685|C0348703|C 0348801|C0348804|C0348806|C0348809|C0348814|C0348968|C0348970|C0349359|C0349468| C0375326|C0477908|C0477909|C0494034|C0494059|C0494086|C0494137|C0494638|C049463 91|C04946401|C0494643| C04946441|C0494675| C0494678| C0494683| C04953 891|C04953901|C05190 301|C06945041|C0869210,Temporal_cortex_TRIM37,1687); (21,C0004626|C0032285|C0032300|C0032302|C0032308|C0032310|C0034063|C0152413|C0152487|C0152502|C0155858|C015586 0|C0155862|C0158935|C0158944|C0206062|C0260334|C0264522|C0276026|C0276089|C02761 56|C0276333|C0339951|C0339959|C0339961|C0339969|C0339971|C0343320|C0343323|C0343 324|C0343666|C0348152|C0348161|C0348193|C0348677|C0348678|C0348679|C0348680|C034 8681|C0348684|C0348685|C0348703|C0348801|C0348804|C0348806|C0348809|C0348814|C03 48968|C0348970|C0349359|C0349468|C0375326|C0477908|C0477909|C0494034|C0494059|C0 494086| C0494137|C04946381|C04946391|C04946401|C0494643| C04946441|C0494675| C0494678|C 0494683|C0495389|C0495390|C0519030|C0694504|C0869210,Colorectum_VPS37B,1656); (21, C0004626|C0032285|C0032300|C0032302|C0032308|C0032310|C0034063|C0152413|C015248 7|C0152502|C0155858|C0155860|C0155862|C0158935|C0158944|C0206062|C0260334|C02645 22|C0276026|C0276089|C0276156|C0276333|C0339951|C0339959|C0339961|C0339969|C0339 971|C0343320|C0343323|C0343324|C0343666|C0348152|C0348161|C0348193|C0348677|C034 8678|C0348679|C0348680|C0348681|C0348684|C0348685|C0348703|C0348801|C0348804|C03 48806|C0348809|C0348814|C0348968|C0348970|C0349359|C0349468|C0375326|C0477908|C0 4779091|C04940341|C04940591|C0494086| C0494137|C04946381|C04946391|C04946401|C0494643| C 04946441|C0494675|C04946781|C0494683|C04953891|C04953901|C05190301|C06945041|C086921 0, Brain white matter_STON1-GTF2A1 L,1647); (21,C0004626|C00032285|C000323001|C0032302|C00032308|C000323101|C003406 3|C0152413|C0152487|C0152502|C0155858|C0155860|C0155862|C0158935|C0158944|C02060 62|C0260334|C0264522|C0276026|C0276089|C0276156|C0276333|C0339951|C0339959|C0339 961|C0339969|C0339971|C0343320|C0343323|C0343324|C0343666|C0348152|C0348161|C034 8193|C0348677|C0348678|C0348679|C0348680|C0348681|C0348684|C0348685|C0348703|C03 48801|C0348804|C0348806|C0348809|C0348814|C0348968|C0348970|C0349359|C0349468|C0 375326|C0477908|C0477909|C0494034|C0494059|C0494086|C0494137|C0494638|C0494639|C 04946401|C0494643| C04946441|C0494675| C04946781|C0494683| C04953 891|C04953901|C0519030|C06945041|C0869210,Endometrial tissue_TMEM159,1635); (21,C0004626|C0032285|C0032300|C0032302|C0032308|C0032310|C 0034063|C0152413|C0152487|C0152502|C0155858|C0155860|C0155862|C0158935|C0158944| C0206062|C0260334|C0264522|C0276026|C0276089|C0276156|C0276333|C0339951|C033995 9|C0339961|C0339969|C0339971|C0343320|C0343323|C0343324|C0343666|C0348152|C03481 61|C0348193|C0348677|C0348678|C0348681|C0348684|C0348685|C0348 703|C0348801|C0348804|C0348806|C0348809|C0348814|C0348968|C0348970|C0349359|C034 9468|C0375326|C0477908|C0477909|C0494034|C0494059|C0494086|C0494137|C0494638|C04 94639|C0494640|C0494643|C0494644|C0494675|C0494678|C0494683|C0495389|C0495390|C0 519030|C0694504|C0869210,Endometrial tissue_GLB1,1550); (21,C0004626|C0032285|C0032300|C0032302|C0032308|C0032310|C0034 063|C0152413|C0152487|C0152502|C0155858|C0155860|C0155862|C0158935|C0158944|C020 6062|C0260334|C0264522|C0276026|C0276089|C0276156|C0276333|C0339951|C0339959|C03 39961|C0339969|C0339971|C0343320|C0343323|C0343324|C0343666|C0348152|C0348161|C0 348193|C0348677|C0348 3679|C03486801|C0348681|C0348684|C0348685|C0348703|C 0348801|C0348804|C0348806|C0348809|C0348814|C0348968|C0348970|C0349359|C0349468| C0375326|C0477908|C0477909|C0494034|C0494059|C0494086|C0494137|C0494638|C049463 91|C04946401|C04946431|C0494644|C0494675|C04946780|C0494683|C0495389|C0495390|C05190 30|C0694504|C0869210,Monocyte_PDHX,1494); (21,C0004626|C0032285|C0032300|C0032302|C0032308|C0032310|C0034063|C0152413|C0152487|C0152502|C0155858|C0155860|C015586 2|C0158935|C0158944|C0206062|C0260334|C0264522|C0276026|C0276089|C0276156|C02763 33|C0339951|C0339959|C0339961|C0339969|C0339971|C0343320|C0343323|C0343324|C0343 666|C0348152|C0348161|C0348193|C0348677|C0348678|C0348679|C0348680|C0348681|C034 8684|C0348685|C0348703|C0348801|C0348804|C0348806|C0348809|C0348814|C0348968|C03 48970|C0349359|C0349468|C0375326|C0477908|C0477909|C0494034|C0494059|C0494086|C0 494137|C0494638|C0494639|C0494640|C0494643|C0494644|C0494675|C0494678|C0494683|C 0495389|C0495390|C0519030|C0694504|C0869210,Bladder_THEM6,1490); (21,C0004626|C00 32285|C0032300|C0032302|C0032308|C0032310|C0034063|C0152413|C0152487|C0152502|C0 155858|C0155860|C0155862|C0158935|C0158944|C0206062|C0260334|C0264522|C0276026|C 0276089|C0276156|C0276333|C0339951|C0339959|C0339961|C0339969|C0339971|C0343320| C0343323|C0343324|C0343666|C0348152|C0348161|C0348193|C0348677|C0348678|C034867 9|C0348680|C0348681|C0348684|C0348685|C0348703|C0348801|C0348804|C0348806|C03488 09|C0348814|C0348968|C0348970|C0349359|C0349468|C0375326|C0477908|C0477909|C0494 034|C0494059|C0494086|C0494137|C0494638|C0494639|C0494640|C0494643|C0494644|C049 4675|C0494678|C0494683|C0495389|C0495390|C0519030|C0694504|C0869210,Temporal_cort ex_MYADML2,1475); (21,C0004626|C0032285|C0032300|C0032302|C0032308|C0032310|C00 34063|C0152413|C0152487|C0152502|C0155858|C0155860|C0155862|C0158935|C0158944|C0 206062|C0260334|C0264522|C0276026|C0276089|C0276156|C0276333|C0339951|C0339959|C 0339961|C0339969|C0339971|C0343320|C0343323|C0343324|C0343666|C0348152|C0348161| C0348193|C0348677|C0348678|C0348679|C0348680|C0348681|C0348684|C0348685|C034870 3|C0348801|C0348804|C0348806|C0348809|C0348814|C0348968|C0348970|C0349359|C03494 68|C0375326|C0477908|C0477909|C0494034|C0494059|C0494086|C0494137|C0494638|C0494 6391|C04946401|C0494643| C04946441|C0494675| C0494678| C0494683| C04953891|C04953901|C051 90301|C06945041|C0869210,Brain white m atter_CLEC12A,1473); (21,C0004626|C0032285|C0032300|C0032302|C0032308|C0032310|C 0034063|C0152413|C0152487|C0152502|C0155858|C0155860|C0155862|C0158935|C0158944| C0206062|C0260334|C0264522|C0276026|C0276089|C0276156|C0276333|C0339951|C033995 9|C0339961|C0339969|C0339971|C0343320|C0343323|C0343324|C0343666|C0348152|C03481 61|C0348193|C0348677|C0348678|C0348681|C0348684|C0348685|C0348 703|C0348801|C0348804|C0348806|C0348809|C0348814|C0348968|C0348970|C0349359|C034 9468|C0375326|C0477908|C0477909|C0494034|C0494059|C0494086|C0494137|C0494638|C04 94639|C0494640|C0494643|C0494644|C0494675|C0494683|C0495389|C0495390|C0 519030|C0694504|C0869210,Macrophage_MEPE,1424); (21,C0004626|C0032285|C0032300|C0 032302|C0032308|C0032310|C0034063|C0152413|C0152487|C0152502|C0155858|C0155860|C 0155862|C0158935|C0158944|C0206062|C0260334|C0264522|C0276026|C0276089|C0276156| C0276333|C0339951|C0339959|C0339961|C0339969|C0339971|C0343320|C0343323|C034332 4|C0343666|C0348152|C0348161|C0348193|C0348677|C0348678|C0348679|C0348680|C03486 81|C0348684|C0348685|C0348703|C0348801|C0348804|C0348806|C0348809|C0348814|C0348 968|C0348970|C0349359|C0349468|C0375326|C0477908|C0477909|C0494034|C0494059|C049 4086| C04941371|C0494638|1|C04946391|C04946401|C0494643|1|C0494644|C0494675|1|C0494678|1|C04 94683|C04953891|C04953901|C05190301|C06945041|C0869210,Endometrial tissue_PODN,1416); (25,C0006267|C0152239|C0348809|C0348968|C0431530|C0494034|C0494 659,Whole_blood_AKIRIN2,2911); (25,C0006267|C0152239|C0348809|C0348968|C0431530|C 04940341|C0494659,Monocyte_PER2,2255); (25,C0006267|C01522391|C03488091|C0348968|C04 31530|C0494034|C0494659,Colon_RAMP2,2035); (25,C0006267|C0152239|C0348809|C034896 8|C0431530|C0494034|C0494659,Unknown_CD86,1905); (25,C0006267|C0152239|C0348809|C 0348968|C0431530|C0494034|C0494659,Monocyte_RILP,1893); (25,C0006267|C0152239|C034 8809|C0348968|C0431530|C0494034|C0494659,Monocyte_SNX9,1875); (25,C0006267|C01522 391|C03488091|C0348968|C04315301|C04940341|C0494659,Endometrial tissue_TVP23CP2,1868); (25,C0006267|C0152239|C0348809|C0348968|C0431530|C0494034|C 0494659,Monocyte_EIF3G,1705); (25,C0006267|C0152239|C0348809|C0348968|C0431530|C04 94034|C0494659,Endometrial tissue_SMC2,1699); (25,C0006267|C0152239|C0348809|C0348968|C0431530|C0494034|C0494 659,Temporal_cortex_TRIM37,1687); (25,C0006267|C0152239|C0348809|C0348968|C0431530| C04940341|C0494659,Colorectum_VPS37B,1656); (25,C0006267|C01522391|C03488091|C034896 8|C04315301|C04940341|C0494659,Brain white matter_STON1-GTF2A1L,1647); (25,C0006267|C0152239|C0348809|C0348968|C0431530|C0494034|C049465 9,Endometrial tissue_TMEM159,1635); (25,C0006267|C0152239|C0348809|C0348968|C0431530|C0494034|C 0494659,Endometrial tissue_GLB1,1550); (25,C0006267|C0152239|C0348809|C0348968|C0431530|C0494034|C0494 659,Monocyte_PDHX,1494); (25,C0006267|C0152239|C0348809|C0348968|C0431530|C049403 4|C0494659,Bladder_THEM6,1490); (25,C0006267|C0152239|C0348809|C0348968|C0431530| C0494034|C0494659,Temporal_cortex_MYADML2,1475); (25,C0006267|C0152239|C0348809|C03489681|C04315301|C04940341|C0494659,Brain white matter_CLEC12A,1473); (25,C0006267|C0152239|C0348809|C0348968|C0431530|C0494034|C 0494659,Macrophage_MEPE,1424); (25,0006267| C0152239|C0348809|C0348968|C0431530|C 0494034|C0494659,Endometrial tissue_PODN,1416); (29,C0007820|C0013537|C0017614|C0018801|C0020538|C0020541|C0020 545|C0022661|C0031190|C0033845|C0035078|C0085580|C0151744|C0152105|C0152171|C015 5616|C0155711|C0348586|C0348587|C0348752|C0348859|C0349368|C0392682|C0393561|C04 52204|C0495174|C0495176|C0495177|C0495178|C0495180|C0495183|C0848548|C3495403|C3 648968,Whole_blood_AKIRIN2,2911); (29,C0007820|C0013537|C0017614|C0018801|C002053 8|C0020541|C00205451|C0022661|C0031190|C0033845|C0035078|C00855801|C0151744|C01521 05|C0152171|C0155616|C0155711|C0348586|C0348587|C0348752|C0348859|C0349368|C0392 682|C0393561|C0452204|C0495174|C0495176|C0495177|C0495178|C0495180|C0495183|C084 8548|C3495403|C3648968,Monocyte_PER2,2255); (29,C0007820|C0013537|C0017614|C00188 01|C0020538|C0020541|C0020545|C0022661|C0031190|C0033845|C0035078|C0085580|C0151 744|C0152105|C0152171|C0155616|C0155711|C0348586|C0348587|C0348752|C0348859|C034 9368|C0392682|C0393561|C0452204|C0495174|C0495176|C0495177|C0495178|C0495180|C04 95183|C0848548|C3495403|C3648968,Colon_RAMP2,2035); (29,C0007820|C0013537|C001761 41|C001880|C00205381|C0020541|C0020545|C0022661|C00311901|C0033845|C00350781|C00855 80|C0151744|C0152105|C0152171|C0155616|C0155711|C0348586|C0348587|C0348752|C0348 859|C0349368|C0392682|C0393561|C0452204|C0495174|C0495176|C0495177|C0495178|C049 5180|C0495183|C0848548|C3495403|C3648968,Unknown_CD86,1905); (29,C00078201|C00135 37|C0017614|C0018801|C0020538|C0020541|C0020545|C0022661|C0031190|C0033845|C0035 078|10085580|C0151744|C0152105|C0152171|C0155616|C0155711|C0348586|C0348587|C034 8752|C0348859|C0349368|C0392682|C0393561|C0452204|C0495174|C0495176|C0495177|C04 95178|C0495180|C0495183|C0848548|C3495403|C3648968,Monocyte_RILP,1893); (29,C00078 20|C0013537|C0017614|C0018801|C0020538|C0020541|C0020545|C0022661|C0031190|C0033 845|C0035078|C0085580|C0151744|C0152105|C0152171|C0155616|C0155711|C0348586|C034 8587|C0348752|C0348859|C0349368|C0392682|C0393561|C0452204|C0495174|C0495176|C04 95177|C0495178|C0495180|C0495183|C0848548|C3495403|C3648968,Monocyte_SNX9,1875); (29,C0007820|C0013537|C0017614|C0018801|C0020538|C0020541|C0020545|C0022661|C003 1190|C0033845|C0035078|C0085580|C0151744|C0152105|C0152171|C0155616|C0155711|C03 48586|C0348587|C0348752|C0348859|C0349368|C0392682|C0393561|C0452204|C0495174|C0 495176|C0495177|C0495178|C0495180|C0495183|C0848548|C3495403|C3648968,Endometrial tissue_TVP23CP2,1868); (29,C0007820|C0013537|C0017614|C0018801|C0020538|C0020541|C 0020545|C00022661|C0031190|C0033845|C0035078|C0085580|C0151744|C0152105|C0152171| C0155616|C0155711|C0348586|C0348587|C0348752|C0348859|C0349368|C0392682|C039356 1|C0452204|C0495174|C0495176|C0495177|C0495178|C0495180|C0495183|C0848548|C34954 03|C3648968,Monocyte_EIF3G,1705); (29,C0007820|C0013537|C0017614|C0018801|C0020538|C0020541|C00205451|C0022661|C0031190|C0033845|C0035078|C00855801|C0151744|C015210 5|C0152171|C0155616|C0155711|C0348586|C0348587|C0348752|C0348859|C0349368|C03926 82|C0393561|C0452204|C0495174|C0495176|C0495177|C0495178|C0495180|C0495183|C0848 548|C3495403|C3648968,Endometrial tissue_SMC2,1699); (29,C0007820|C0013537|C0017614|C0018801|C0020538|C0020541|C0020 545|C0022661|C0031190|C0033845|C0035078|C0085580|C0151744|C0152105|C0152171|C015 5616|C0155711|C0348586|C0348587|C0348752|C0348859|C0349368|C0392682|C0393561|C04 52204|C0495174|C0495176|C0495177|C0495178|C0495180|C0495183|C0848548|C3495403|C3 648968,Temporal_cortex_TRIM37,1687); (29,C00078201|C0013537|C00176141|C0018801|C0020 538|C0020541|C0020545|C0022661|C0031190|C0033845|C0035078|C0085580|C0151744|C015 2105|C0152171|C0155616|C0155711|C0348586|C0348587|C0348752|C0348859|C0349368|C03 92682|C0393561|C0452204|C0495174|C0495176|C0495177|C0495178|C0495180|C0495183|C0 848548|C3495403|C3648968,Colorectum_VPS37B,1656); (29,C0007820|C0013537|C0017614|C 0018801|C0020538|C0020541|C0020545|C0022661|C0031190|C0033845|C0035078|C0085580| C0151744|C0152105|C0152171|C0155616|C0155711|C0348586|C0348587|C0348752|C034885 9|C0349368|C0392682|C0393561|C0452204|C0495174|C0495176|C0495177|C0495178|C04951 801|C0495183|C0848548|C3495403|C3648968,Brain white matter_STON1-5|C0022661|C0031190|C0033845|C0035078|C0085580|C0151744|C0152105|C0152171|C01556 16|C0155711|C0348586|C0348587|C0348752|C0348859|C0349368|C0392682|C0393561|C0452 204|C0495174|C0495176|C0495177|C0495178|C0495180|C0495183|C0848548|C3495403|C364 8968,Endometrial tissue_TMEM159,1635); (29,C0007820|C0013537|C0017614|C0018801|C0020538|C0020541|C 0020545|C00022661|C0031190|C0033845|C0035078|C0085580|C0151744|C0152105|C0152171| C0155616|C0155711|C0348586|C0348587|C0348752|C0348859|C0349368|C0392682|C039356 1|C0452204|C0495174|C0495176|C0495177|C0495178|C0495180|C0495183|C0848548|C34954 03|C3648968,Endometrial tissue_GLB1,1550); (29,C0007820|C0013537|C0017614|C0018801|C0020538|C0020541|C0020 545|C0022661|C0031190|C0033845|C0035078|C0085580|C0151744|C0152105|C0152171|C015 5616|C0155711|C0348586|C0348587|C0348752|C0348859|C0349368|C0392682|C0393561|C04 52204|C0495174|C0495176|C0495177|C0495178|C0495180|C0495183|C0848548|C3495403|C3 648968,Monocyte_PDHX,1494); (29,C00078201|C0013537|C00176141|C0018801|C0020538|C002 0541|C0020545|C0022661|C0031190|C0033845|C0035078|C0085580|C0151744|C0152105|C01 52171|C0155616|C0155711|C0348586|C0348587|C0348752|C0348859|C0349368|C0392682|C0 393561|C0452204|C0495174|C0495176|C0495177|C0495178|C0495180|C0495183|C0848548|C 3495403|C3648968,Bladder_THEM6,1490); (29,C0007820|C0013537|C0017614|C0018801|C00 20538|C0020541|C0020545|C0022661|C0031190|C0033845|C0035078|C0085580|C0151744|C0 152105|C0152171|C0155616|C0155711|C0348586|C0348587|C0348752|C0348859|C0349368|C 0392682|C0393561|C0452204|C0495174|C0495176|C0495177|C0495178|C0495180|C0495183| C0848548|C3495403|C3648968,Temporal_cortex_MYADML2,1475); (29,C0007820|C0013537| C0017614|C0018801|C0020538|C0020541|C0020545|C0022661|C0031190|C0033845|C003507 81|C0085580|C0151744|C0152105|C0152171|C0155616|C0155711|C0348586|C0348587|C03487 52|C0348859|C0349368|C0392682|C0393561|C0452204|C0495174|C0495176|C0495177|C0495 1781|C04951801|C0495183|C0848548|C3495403|C3648968,Brain white matter_CLEC12A,1473); (29,C0007820|C0013537|C0017614|C0018801|C0020538|C0020541|C 0020545|C0022661|C0031190|C0033845|C0035078|C0085580|C0151744|C0152105|C0152171| C0155616|C0155711|C0348586|C0348587|C0348752|C0348859|C0349368|C0392682|C039356 1|C0452204|C0495174|C0495176|C0495177|C0495178|C0495180|C0495183|C0848548|C34954 03|C3648968,Macrophage_MEPE,1424); (29,C0007820|C0013537|C0017614|C0018801|C00205 38|C0020541|C0020545|C0022661|C0031190|C0033845|C0035078|C0085580|C0151744|C0152 105|C0152171|C0155616|C0155711|C0348586|C0348587|C0348752|C0348859|C0349368|C039 485481|C3495403|C3648968,Endometrial tissue_PODN,1416); (30,C0007860|C0014719|C0348904|C0477866|C0494054,Whole_blood_A KIRIN2,2911); (30,C00078601|C0014719|C0348904|C0477866|C0494054,Monocyte_PER2,2255); (30,C00078601|C00147191|C03489041|C0477866|C0494054,Colon_RAMP2,2035); (30,C0007860|C00014719|C0348904|C0477866|C0494054,Unknown_CD86,1905); (30,C0007860|C0014719|C0 348904|C0477866|C0494054,Monocyte_RILP,1893); (30,C0007860|C0014719|C0348904|C0477 866|C0494054,Monocyte_SNX9,1875); (30,C00078601|C0014719|C0348904|C0477866|C049405 4,Endometrial tissue_TVP23CP2,1868); (30,C0007860|C0014719|C0348904|C0477866|C0494054,Monocyte_E IF3G,1705); (30,C0007860|C0014719|C0348904|C0477866|C0494054,Endometrial tissue_SMC2,1699); (30,C0007860|C0014719|C0348904|C0477866|C0494054,Temporal_cortex_TRIM37,1687); (30,C0007860|C0014719|C0348904|C0477866|C0494054,Colorectum_VPS37 B,1656); (30,C00078601|C00147191|C03489041|C0477866|C0494054,Brain white matter_STON1-GTF2A1L,1647); (30,C000078601|C0014719|C0348904|C0477866|C0494054,Endometrial tissue_TMEM159,1635); (30,C0007860|C0014719|C0348904|C0477866|C0494054,Endometrial tissue_GLB1,1550); (30,C0007860|C0014719|C0348904|C0477866|C0494054,Monocyte_PDHX ,1494); (30,C0007860|C0014719|C0348904|C0477866|C0494054,Bladder_THEM6,1490); (30,C0 007860|C0014719|C0348904|C0477866|C0494054,Temporal_cortex_MYADML2,1475); (30,C0 0078601|C00147191|C03489041|C0477866|C0494054,Brain white matter_CLEC12A,1473); (30,C00078601|C0014719|C0348904|C0477866|C0494054,Macrophage_MEPE,1424); (30,C0007860|C0014719|C0348904|C0477866|C0494054,Endometrial tissue_PODN,1416); (31,C0007871|C00118491|C00118541|C00118601|C0012242|C00127391|C0013 537|C0013927|C0018790|C0018801|C0019693|C0020450|C0020538|C0021361|C0024528|C002 74981|C0027765|C0028962|C0029496|C0029604|C0031154|C0032969|C0032984|C0032987|C00 32989|C0032991|C0032993|C0032994|C0033010|C0034065|C0035078|C0035204|C00369861|C0 038843|C00390701|C0039236|C0080276|C0085207|C0085740|C0152150|C0152151|C0152152|C 0152964|C0155749|C0155774|C0155778|C0155796|C0156373|C0156543|C0156604|C0156677| C0156699|C0156703|C0156756|C0157535|C0158806|C0158816|C0161754|C0178269|C017828 8|C0178298|C0233214|C0242172|C0260550|C0260551|C0263660|C0269294|C0269661|C02696 72|C0271641|C0275667|C0275820|C0302487|C0332544|C0339951|C0340747|C0342044|C0342 054|C0342068|C0348447|C0348786|C0348859|C0348877|C0348881|C0348950|C0349253|C036 2068|C0391989|C0392070|C0405328|C0411164|C0411169|C0451717|C0451783|C0451790|C04 51791|C0451792|C0451799|C0451800|C0451801|C0451802|C0451803|C0451810|C0451814|C0 0476570|C0476572|C0476635|C0477322|C0477391|C0477762|C0477808|C0477809|C0477810| C0477811|C0477812|C0477813|C0477814|C0477815|C0477816|C0477817|C0477819|C047782 O|C0477865|C0477876|C0477878|C0477881|C0477885|C0478521|C0478618|C0494048|C04944 50|C0494514|C0494598|C0494783|C0495168|C0495169|C0495170|C0495171|C0495172|C0495 173|C0495174|C0495175|C0495176|C0495177|C0495180|C0495184|C0495187|C0495188|C049 5189|C0495190|C0495192|C0495196|C0495197|C0495198|C0495199|C0495224|C0495225|C04 95227|C0495251|C0495292|C0495301|C0495302|C0495304|C0495308|C0495309|C0495310|C0 495311|C0495312|C0495313|C0495314|C0495315|C0495316|C0495317|C0495318|C0495351|C 0495699|C0495706|C0496132|C0496136|C0496158|C049664|C0496642|C0496643|C0496644|C0496645|C0496646|C0496693|C0496727|C0694449|C0694451|C0694452|C0694454|C069445 6|C0700573|C0728889|C0728936|C0851207|C0851208|C0851209|C0851217|C0869210|C08692 72|C0878544|C1314803|C1367972|C1533618|C1533626|C1704300|C3146283|C3648968,Whole_blood_AKIRIN2,2911); (31,C0007871|C0011849|C0011854|C00118601|C0012242|C0012739|C 0013537|C0013927|C0018790|C0018801|C0019693|C0020450|C0020538|C0021361|C0024528| C0027498|C0027765|C0028962|C0029496|C0029604|C0031154|C0032969|C0032984|C003298 7|C0032989|C0032991|C0032993|C0032994|C0033010|C0034065|C0035078|C0035204|C00369 86|C0038843|C0039070|C0039236|C0080276|C0085207|C0085740|C0152150|C0152151|C0152 152|C0152964|C0155749|C0155774|C0155778|C0155796|C0156373|C0156543|C0156604|C015 6677|C0156699|C0156703|C0156756|C0157535|C0158806|C0158816|C0161754|C0178269|C01 78288|C0178298|C0233214|C0242172|C0260550|C0260551|C0263660|C0269294|C0269661|C0 269672|C0271641|C0275667|C0275820|C0302487|C0332544|C0339951|C0340747|C0342044|C 0342054|C0342068|C0348447|C0348786|C0348859|C0348877|C0348881|C0348950|C0349253| C03620681|C0391989|C0392070|C0405328|C0411164|C0411169|C0451717|C0451783|C045179 0|C0451791|C0451792|C0451799|C0451800|C0451801|C0451802|C0451803|C0451810|C04518 14|C0451816|C0451822|C0452164|C0452165|C0475523|C0475578|C0476442|C0476491|C0476 550|C0476570|C0476572|C0476635|C0477322|C0477391|C0477762|C0477808|C0477809|C047 7810|C0477811|C0477812|C0477813|C0477814|C0477815|C0477816|C0477817|C0477819|C04 77820|C0477865|C0477876|C0477878|C0477881|C0477885|C0478521|C0478618|C0494048|C0 494450|C0494514|C0494598|C0494783|C0495168|C0495169|C0495170|C0495171|C0495172|C 0495173|C0495174|C0495175|C0495176|C0495177|C0495180|C0495184|C0495187|C0495188| C0495189|C0495190|C0495192|C0495196|C0495197|C0495198|C0495199|C0495224|C049522 5|C0495227|C0495251|C0495292|C0495301|C0495302|C0495304|C0495308|C0495309|C04953 10|C0495311|C0495312|C0495313|C0495314|C0495315|C0495316|C0495317|C0495318|C0495 351|C0495699|C0495706|C0496132|C0496136|C0496158|C0496641|C0496642|C0496643|C049 6644|C04966451|C0496646|C0496693|C0496727|C0694449|C0694451|C0694452|C0694454|C06 94456|C0700573|C0728889|C0728936|C0851207|C0851208|C0851209|C0851217|C0869210|C0 869272|C0878544|C1314803|C1367972|C1533618|C1533626|C1704300|C3146283|C3648968, Monocyte_PER2,2255); (31,C0007871|C0011849|C0011854|C0011860|C0012242|C0012739|C0 013537|C0013927|C0018790|C0018801|C0019693|C0020450|C0020538|C0021361|C0024528|C 0027498|C0027765|C0028962|C0029496|C0029604|C0031154|C0032969|C0032984|C0032987| C0032989|C0032991|C0032993|C0032994|C0033010|C0034065|C0035078|C0035204|C003698 6|C0038843|C0039070|C0039236|C00802761|C0085207|C0085740|C0151|C0152151|C01521 52|C0152964|C0155749|C0155774|C0155778|C0155796|C0156373|C0156543|C0156604|C0156 677|C0156699|C0156703|C0156756|C0157535|C0158806|C0158816|C0161754|C0178269|C017 8288|C0178298|C0233214|C0242172|C0260550|C0260551|C0263660|C0269294|C0269661|C02 69672|C0271641|C0275667|C0275820|C0302487|C0332544|C0339951|C0340747|C0342044|C0 3420541|C0342068|C0348447|C03487861|C0348859|C0348877|C0348881|C0348950|C0349253|C 0362068|C0391989|C0392070|C0405328|C0411164|C0411169|C0451717|C0451783|C0451790| C0451791|C0451792|C0451799|C0451800|C0451801|C0451802|C0451803|C0451810|C045181 4|C0451816|C0451822|C0452164|C0452165|C0475523|C0475578|C0476442|C0476491|C04765 50|C0476570|C0476572|C0476635|C0477322|C0477391|C0477762|C0477808|C0477809|C0477 810|C0477811|C0477812|C0477813|C0477814|C0477815|C0477816|C0477817|C0477819|C047 7820|C0477865|C0477876|C0477878|C0477881|C0477885|C0478521|C0478618|C0494048|C04 94450|C0494514|C0494598|C0494783|C0495168|C0495169|C0495170|C0495171|C0495172|C0 495173|C0495174|C0495175|C0495176|C0495177|C0495180|C0495184|C0495187|C0495188|C 0495189|C0495190|C0495192|C0495196|C0495197|C0495198|C0495199|C0495224|C0495225| C0495227|C0495251|C0495292|C0495301|C0495302|C0495304|C0495308|C0495309|C049531 0|C0495311|C0495312|C0495313|C0495314|C0495315|C0495316|C0495317|C0495318|C04953 51|C0495699|C0495706|C0496132|C0496136|C0496158|C0496641|C0496642|C0496643|C0496 644|C0496645|C0496646|C0496693|C0496727|C0694449|C0694451|C0694452|C0694454|C069 4456|C0700573|C0728889|C0728936|C0851207|C0851208|C0851209|C0851217|C0869210|C08 69272|C0878544|C1314803|C1367972|C1533618|C1533626|C1704300|C3146283|C3648968,C0 lon_RAMP2,2035); (31,C0007871|C00118491|C00118541|C00118601|C0012242|C00127391|C00135 37|C0013927|C0018790|C0018801|C0019693|C0020450|C0020538|C0021361|C0024528|C0027 498|C0027765|C0028962|C0029496|C0029604|C0031154|C0032969|C0032984|C0032987|C003 2989|C0032991|C0032993|C0032994|C0033010|C0034065|C0035078|C0035204|C0036986|C00 38843|C0039070|C0039236|C0080276|C0085207|C0085740|C0152150|C0152151|C0152152|C0 0156699|C0156703|C0156756|C0157535|C0158806|C0158816|C0161754|C0178269|C0178288| C0178298|C0233214|C0242172|C0260550|C0260551|C0263660|C0269294|C0269661|C026967 2|C0271641|C0275667|C0275820|C0302487|C0332544|C0339951|C0340747|C0342044|C03420 541|C0342068|C0348447|C03487861|C0348859|C0348877|C0348881|C0348950|C0349253|C0362 068|C0391989|C0392070|C0405328|C0411164|C0411169|C0451717|C0451783|C0451790|C045 1791|C0451792|C0451799|C0451800|C0451801|C0451802|C0451803|C0451810|C0451814|C04 51816|C0451822|C0452164|C0452165|C0475523|C0475578|C0476442|C0476491|C0476550|C0 476570|C0476572|C0476635|C0477322|C0477391|C0477762|C0477808|C0477809|C0477810|C 0477811|C0477812|C0477813|C0477814|C0477815|C0477816|C0477817|C0477819|C0477820| C0477865|C0477876|C0477878|C0477881|C0477885|C0478521|C0478618|C0494048|C049445 0|C0494514|C0494598|C0494783|C0495168|C0495169|C0495170|C0495171|C0495172|C04951 73|C0495174|C0495175|C0495176|C0495177|C0495180|C0495184|C0495187|C0495188|C0495 189|C0495190|C0495192|C0495196|C0495197|C0495198|C0495199|C0495224|C0495225|C049 5227|C0495251|C0495292|C0495301|C0495302|C0495304|C0495308|C0495309|C0495310|C04 95311|C0495312|C0495313|C0495314|C0495315|C0495316|C0495317|C0495318|C0495351|C0 495699|C0495706|C0496132|C0496136|C0496158|C0496641|C0496642|C0496643|C0496644|C 04966451|C0496646|C0496693|C0496727|C0694449|C0694451|C0694452|C0694454|C0694456|C0700573|C0728889|C0728936|C0851207|C0851208|C0851209|C0851217|C0869210|C086927 2|C0878544|C1314803|C1367972|C1533618|C1533626|C1704300|C3146283|C3648968,Unkno wn_CD86,1905); (31,C0007871|C0011849|C0011854|C0011860|C0012242|C0012739|C0013537|C0013927|C0018790|C0018801|C0019693|C0020450|C0020538|C0021361|C0024528|C002749 8|C0027765|C0028962|C0029496|C0029604|C0031154|C0032969|C0032984|C0032987|C00329 89|C0032991|C0032993|C0032994|C0033010|C0034065|C0035078|C0035204|C0036986|C0038 843|C0039070|C0039236|C0080276|C0085207|C0085740|C0152150|C0152151|C0152152|C015 2964|C0155749|C0155774|C0155778|C0155796|C0156373|C0156543|C0156604|C0156677|C01 56699|C0156703|C0156756|C0157535|C0158806|C0158816|C0161754|C0178269|C0178288|C0 178298|C0233214|C0242172|C0260550|C026055|C0263660|C0269294|C026966|C0269672|C 0271641|C0275667|C0275820|C0302487|C0332544|C0339951|C0340747|C0342044|C0342054| C0342068|C0348447|C0348786|C0348859|C0348877|C0348881|C0348950|C0349253|C036206 8|C0391989|C0392070|C0405328|C0411164|C0411169|C0451717|C0451783|C0451790|C04517 91|C0451792|C0451799|C0451800|C0451801|C0451802|C0451803|C0451810|C0451814|C0451 816|C0451822|C0452164|C0452165|C0475523|C0475578|C0476442|C0476491|C0476550|C047 6570|C0476572|C0476635|C0477322|C0477391|C0477762|C0477808|C0477809|C0477810|C04 77811|C0477812|C0477813|C0477814|C0477815|C0477816|C0477817|C0477819|C0477820|C0 477865|C0477876|C0477878|C0477881|C0477885|C0478521|C0478618|C0494048|C0494450|C 0494514|C0494598|C0494783|C0495168|C0495169|C0495170|C0495171|C0495172|C0495173| C0495174|C0495175|C0495176|C0495177|C0495180|C0495184|C0495187|C0495188|C049518 9|C0495190|C0495192|C0495196|C0495197|C0495198|C0495199|C0495224|C0495225|C04952 27|C0495251|C0495292|C0495301|C0495302|C04953041|C0495308|C0495309|C0495310|C0495 311|C0495312|C0495313|C0495314|C0495315|C0495316|C0495317|C0495318|C0495351|C049 5699|C0495706|C0496132|C0496136|C0496158|C0496641|C0496642|C0496643|C0496644|C04 96645|C0496646|C0496693|C0496727|C0694449|C0694451|C0694452|C0694454|C0694456|C0 700573|C0728889|C0728936|C0851207|C0851208|C0851209|C0851217|C0869210|C0869272|C 0878544|C1314803|C1367972|C1533618|C1533626|C1704300|C3146283|C3648968,Monocyte_RILP,1893); (31,C0007871|C0011849|C0011854|C0011860|C0012242|C0012739|C0013537|C00 13927|C0018790|C0018801|C0019693|C0020450|C0020538|C0021361|C0024528|C0027498|C0 027765|C0028962|C0029496|C0029604|C0031154|C0032969|C0032984|C0032987|C0032989|C 0032991|C0032993|C0032994|C0033010|C0034065|C0035078|C0035204|C0036986|C0038843| C0039070|C0039236|C0080276|C0085207|C0085740|C0152150|C0152151|C0152152|C015296 4|C0155749|C0155774|C0155778|C0155796|C0156373|C0156543|C0156604|C0156677|C01566 99|C0156703|C0156756|C0157535|C0158806|C0158816|C0161754|C0178269|C0178288|C0178 298|C02332141|C0242172|C02605501|C0260551|C02636601|C02692941|C0269661|C0269672|C027 1641|C0275667|C0275820|C0302487|C0332544|C0339951|C0340747|C0342044|C0342054|C03 42068|C0348447|C03487861|C0348859|C0348877|C0348881|C0348950|C0349253|C0362068|C0 391989|C0392070|C0405328|C0411164|C0411169|C0451717|C0451783|C0451790|C0451791|C 0451792|C0451799|C0451800|C0451801|C0451802|C0451803|C0451810|C0451814|C0451816| C0451822|C0452164|C0452165|C0475523|C0475578|C0476442|C0476491|C0476550|C047657 0|C0476572|C0476635|C0477322|C0477391|C0477762|C0477808|C0477809|C0477810|C04778 1|C0477812|C0477813|C0477814|C0477815|C0477816|C0477817|C0477819|C0477820|C0477 865|C0477876|C0477878|C0477881|C0477885|C0478521|C0478618|C0494048|C0494450|C049 4514|C0494598|C0494783|C0495168|C0495169|C0495170|C0495171|C0495172|C0495173|C04 95174|C0495175|C0495176|C0495177|C0495180|C0495184|C0495187|C0495188|C0495189|C0 495190|C0495192|C0495196|C0495197|C0495198|C0495199|C0495224|C0495225|C0495227|C 0495251|C0495292|C0495301|C0495302|C0495304|C0495308|C0495309|C0495310|C0495311| C0495312|C0495313|C0495314|C0495315|C0495316|C0495317|C0495318|C0495351|C049569 9|C0495706|C0496132|C0496136|C0496158|C0496641|C0496642|C0496643|C0496644|C04966 45|C0496646|C0496693|C0496727|C0694449|C0694451|C0694452|C0694454|C0694456|C0700 573|C0728889|C0728936|C0851207|C0851208|C0851209|C0851217|C0869210|C0869272|C087 8544|C1314803|C1367972|C1533618|C1533626|C1704300|C3146283|C3648968,Monocyte_SN X9,1875); (31,C0007871|C0011849|C0011854|C0011860|C0012242|C0012739|C0013537|C0013 927|C0018790|C0018801|C0019693|C0020450|C0020538|C0021361|C0024528|C0027498|C002 7765|C0028962|C0029496|C0029604|C0031154|C0032969|C0032984|C0032987|C00329891|C00 32991|C0032993|C0032994|C0033010|C0034065|C0035078|C0035204|C0036986|C0038843|C0 039070|C00392361|C0080276|C00852071|C0085740|C0152150|C0152151|C0152152|C0152964|C 0155749|C0155774|C0155778|C0155796|C0156373|C0156543|C0156604|C0156677|C0156699| C0156703|C0156756|C0157535|C0158806|C0158816|C0161754|C0178269|C0178288|C017829 8|C0233214|C0242172|C0260550|C0260551|C0263660|C0269294|C0269661|C0269672|C02716 41|C0275667|C0275820|C0302487|C0332544|C0339951|C0340747|C0342044|C0342054|C0342 068|C0348447|C0348786|C0348859|C0348877|C0348881|C0348950|C0349253|C0362068|C039 1989|C0392070|C0405328|C0411164|C0411169|C0451717|C0451783|C0451790|C0451791|C04 51792|C0451799|C0451800|C0451801|C0451802|C0451803|C0451810|C0451814|C0451816|C0 451822|C0452164|C0452165|C0475523|C0475578|C0476442|C0476491|C0476550|C0476570|C 0476572|C0476635|C0477322|C0477391|C0477762|C0477808|C0477809|C0477810|C0477811| C0477812|C0477813|C0477814|C0477815|C0477816|C0477817|C0477819|C0477820|C047786 5|C0477876|C0477878|C0477881|C0477885|C0478521|C0478618|C0494048|C0494450|C04945 14|C0494598|C0494783|C0495168|C0495169|C0495170|C0495171|C0495172|C0495173|C0495 174|C0495175|C0495176|C0495177|C0495180|C0495184|C0495187|C0495188|C0495189|C049 5190|C0495192|C0495196|C0495197|C0495198|C0495199|C0495224|C0495225|C0495227|C04 95251|C0495292|C0495301|C0495302|C0495304|C0495308|C0495309|C0495310|C0495311|C0 495312|C0495313|C0495314|C0495315|C0495316|C0495317|C0495318|C0495351|C0495699|C 0495706|C0496132|C0496136|C0496158|C049664|C0496642|C0496643|C0496644|C0496645|C0496646|C0496693|C0496727|C0694449|C0694451|C0694452|C0694454|C0694456|C070057 3|C0728889|C0728936|C0851207|C0851208|C0851209|C0851217|C0869210|C0869272|C08785 44|C1314803|C1367972|C1533618|C1533626|C1704300|C3146283|C3648968,Endometrial tissue_TVP23CP2,1868); (31,C0007871|C0011849|C0011854|C0011860|C0012242|C0012739|C 0013537|C0013927|C0018790|C0018801|C0019693|C0020450|C0020538|C0021361|C0024528| C0027498|C0027765|C0028962|C0029496|C0029604|C0031154|C0032969|C0032984|C003298 7|C0032989|C0032991|C0032993|C0032994|C0033010|C0034065|C0035078|C0035204|C00369 86|C0038843|C0039070|C0039236|C0080276|C0085207|C0085740|C0152150|C0152151|C0152 152|C0152964|C0155749|C0155774|C0155778|C0155796|C0156373|C0156543|C0156604|C015 6677|C0156699|C0156703|C0156756|C0157535|C0158806|C0158816|C0161754|C0178269|C01 269672|C0271641|C0275667|C0275820|C0302487|C0332544|C0339951|C0340747|C0342044|C 0342054|C0342068|C0348447|C0348786|C0348859|C0348877|C0348881|C0348950|C0349253| C0362068|C0391989|C0392070|C0405328|C0411164|C0411169|C0451717|C0451783|C045179 0|C0451791|C0451792|C0451799|C0451800|C0451801|C0451802|C04518039|C0451810|C04518 14|C0451816|C0451822|C0452164|C0452165|C0475523|C0475578|C0476442|C0476491|C0476 550|C0476570|C0476572|C0476635|C0477322|C0477391|C0477762|C0477808|C0477809|C047 7810|C0477811|C0477812|C0477813|C0477814|C0477815|C0477816|C0477817|C0477819|C04 77820|C0477865|C0477876|C0477878|C0477881|C0477885|C0478521|C0478618|C0494048|C0 494450|C0494514|C0494598|C0494783|C0495168|C0495169|C0495170|C0495171|C0495172|C 0495173|C0495174|C0495175|C0495176|C0495177|C0495180|C0495184|C0495187|C0495188| C0495189|C0495190|C0495192|C0495196|C0495197|C0495198|C0495199|C0495224|C049522 5|C0495227|C0495251|C0495292|C0495301|C0495302|C0495304|C0495308|C0495309|C04953 10|C0495311|C0495312|C0495313|C0495314|C0495315|C0495316|C0495317|C0495318|C0495 351|C0495699|C0495706|C0496132|C0496136|C0496158|C0496641|C0496642|C0496643|C049 66441|C0496645|C0496646|C0496693|C0496727|C0694449|C0694451|C0694452|C0694454|C06 94456|C0700573|C0728889|C0728936|C0851207|C0851208|C0851209|C0851217|C0869210|C0 869272|C0878544|C1314803|C1367972|C1533618|C1533626|C1704300|C3146283|C3648968, Monocyte_EIF3G,1705); (31,C0007871|C0011849|C0011854|C0011860|C0012242|C0012739|C 0013537|C0013927|C0018790|C0018801|C0019693|C0020450|C0020538|C0021361|C0024528| C0027498|C0027765|C0028962|C0029496|C0029604|C0031154|C0032969|C0032984|C003298 7|C0032989|C0032991|C0032993|C0032994|C0033010|C0034065|C0035078|C0035204|C00369 86|C0038843|C0039070|C0039236|C0080276|C0085207|C0085740|C0152150|C0152151|C0152 152|C0152964|C0155749|C0155774|C0155778|C0155796|C0156373|C0156543|C0156604|C015 6677|C0156699|C0156703|C0156756|C0157535|C0158806|C0158816|C0161754|C0178269|C01 78288|C0178298|C0233214|C0242172|C0260550|C0260551|C0263660|C0269294|C0269661|C0 269672|C0271641|C0275667|C0275820|C0302487|C0332544|C0339951|C0340747|C0342044|C 0342054|C0342068|C0348447|C0348786|C0348859|C0348877|C0348881|C0348950|C0349253| C03620681|C0391989|C0392070|C0405328|C0411164|C0411169|C0451717|C045179 0|C0451791|C0451792|C0451799|C0451800|C0451801|C0451802|C0451803|C0451810|C04518 14|C0451816|C0451822|C0452164|C0452165|C0475523|C0475578|C0476442|C0476491|C0476 550|C0476570|C0476572|C0476635|C0477322|C0477391|C0477762|C0477808|C0477809|C047 7810|C0477811|C0477812|C0477813|C0477814|C0477815|C0477816|C0477817|C0477819|C04 77820|C0477865|C0477876|C0477878|C0477881|C0477885|C0478521|C0478618|C0494048|C0 C0495189|C0495190|C0495192|C0495196|C0495197|C0495198|C0495199|C0495224|C049522 5|C0495227|C0495251|C0495292|C0495301|C0495302|C0495304|C0495308|C0495309|C04953 10|C0495311|C0495312|C0495313|C0495314|C0495315|C0495316|C0495317|C0495318|C0495 351|C0495699|C0495706|C0496132|C0496136|C0496158|C0496641|C0496642|C0496643|C049 66441|C0496645|C0496646|C0496693|C0496727|C0694449|C0694451|C0694452|C0694454|C06 94456|C0700573|C0728889|C0728936|C0851207|C0851208|C0851209|C0851217|C0869210|C0 869272|C0878544|C1314803|C1367972|C1533618|C1533626|C1704300|C3146283|C3648968,E ndometrial tissue_SMC2,1699); (31,C0007871|C0011849|C0011854|C0011860|C0012242|C0012739|C0013 537|C0013927|C0018790|C0018801|C0019693|C0020450|C0020538|C0021361|C0024528|C002 7498|C0027765|C0028962|C0029496|C0029604|C0031154|C0032969|C0032984|C0032987|C00 32989|C0032991|C0032993|C0032994|C0033010|C0034065|C0035078|C0035204|C0036986|C0 038843|C0039070|C0039236|C0080276|C0085207|C0085740|C0152150|C0152151|C0152152|C 0152964|C0155749|C0155774|C0155778|C0155796|C0156373|C0156543|C0156604|C0156677| C0156699|C0156703|C0156756|C0157535|C0158806|C0158816|C0161754|C0178269|C017828 8|C0178298|C0233214|C0242172|C0260550|C0260551|C0263660|C0269294|C0269661|C02696 72|C0271641|C0275667|C0275820|C0302487|C0332544|C0339951|C0340747|C0342044|C0342 054|C0342068|C0348447|C0348786|C0348859|C0348877|C0348881|C0348950|C0349253|C036 2068|C0391989|C0392070|C0405328|C0411164|C0411169|C0451717|C0451783|C0451790|C04 51791|C0451792|C0451799|C0451800|C0451801|C0451802|C0451803|C0451810|C0451814|C0 451816|C0451822|C0452164|C0452165|C0475523|C0475578|C0476442|C0476491|C0476550|C 0476570|C0476572|C0476635|C0477322|C0477391|C0477762|C0477808|C0477809|C0477810| C0477811|C0477812|C0477813|C0477814|C0477815|C0477816|C0477817|C0477819|C047782 0|C0477865|C0477876|C0477878|C0477881|C0477885|C0478521|C0478618|C0494048|C04944 50|C0494514|C0494598|C0494783|C0495168|C0495169|C0495170|C0495171|C0495172|C0495 173|C0495174|C0495175|C0495176|C0495177|C0495180|C0495184|C0495187|C0495188|C049 5189|C0495190|C0495192|C0495196|C0495197|C0495198|C0495199|C0495224|C0495225|C04 95227|C0495251|C0495292|C0495301|C0495302|C0495304|C0495308|C0495309|C0495310|C0 495311|C0495312|C0495313|C0495314|C0495315|C0495316|C0495317|C0495318|C0495351|C 0495699|C0495706|C0496132|C0496136|C0496158|C049664|C0496642|C0496643|C0496644|C0496645|C0496646|C0496693|C0496727|C0694449|C0694451|C0694452|C0694454|C069445 6|C0700573|C0728889|C0728936|C0851207|C0851208|C0851209|C0851217|C0869210|C08692 oral_cortex_TRIM37,1687); (31,C0007871|C0011849|C0011854|C0011860|C0012242|C001273 9|C0013537|C0013927|C0018790|C0018801|C0019693|C0020450|C0020538|C0021361|C00245 28|C0027498|C0027765|C0028962|C0029496|C0029604|C0031154|C0032969|C0032984|C0032 987|C0032989|C0032991|C0032993|C0032994|C0033010|C0034065|C0035078|C0035204|C003 6986|C0038843|C0039070|C0039236|C0080276|C0085207|C0085740|C015215 1|C01 52152|C0152964|C0155749|C0155774|C0155778|C0155796|C0156373|C0156543|C0156604|C0 156677|C0156699|C0156703|C0156756|C0157535|C0158806|C0158816|C0161754|C0178269|C 01782881|C01782981|C02332141|C0242172|C02605501|C0260551|C02636601|C02692941|C0269661|C0269672|C0271641|C0275667|C0275820|C0302487|C0332544|C0339951|C0340747|C034204 4|C0342054|C0342068|C0348447|C0348786|C0348859|C0348877|C0348881|C0348950|C03492 53|C0362068|C0391989|C0392070|C0405328|C0411164|C0411169|C0451717|C0451783|C0451 790|C0451791|C0451792|C0451799|C0451800|C0451801|C0451802|C0451803|C0451810|C045 1814|C0451816|C0451822|C0452164|C0452165|C0475523|C0475578|C0476442|C0476491|C04 76550|C0476570|C0476572|C0476635|C0477322|C0477391|C0477762|C0477808|C0477809|C0 4778101|C0477811|C0477812|C0477813|C0477814|C0477815|C0477816|C0477817|C0477819|C 04778201|C0477865|C0477876|C04778781|C047788|C0477885|C0478521|C04786181|C0494048|C0494450|C0494514|C0494598|C0494783|C0495168|C0495169|C0495170|C0495171|C049517 2|C0495173|C0495174|C0495175|C0495176|C0495177|C0495180|C0495184|C0495187|C04951 88|C0495189|C0495190|C0495192|C0495196|C0495197|C0495198|C0495199|C0495224|C0495 225|C04952271|C0495251|C0495292|C0495301|C0495302|C04953041|C04953081|C04953091|C049 5310|C0495311|C0495312|C0495313|C0495314|C0495315|C0495316|C0495317|C0495318|C04 95351|C0495699|C0495706|C0496132|C0496136|C0496158|C0496641|C0496642|C0496643|C0 496644|C0496645|C0496646|C0496693|C0496727|C0694449|C069445|C0694452|C0694454|C 0694456|C0700573|C0728889|C0728936|C0851207|C0851208|C0851209|C0851217|C0869210| C0869272|C0878544|C1314803|C1367972|C1533618|C1533626|C1704300|C3146283|C364896 8,Colorectum_VPS37B,1656); (31,C0007871|C00118491|C00118541|C00118601|C0012242|C00127 39|C0013537|C0013927|C0018790|C0018801|C0019693|C0020450|C0020538|C0021361|C0024 528|C0027498|C0027765|C0028962|C0029496|C0029604|C0031154|C0032969|C0032984|C003 2987|C0032989|C0032991|C0032993|C0032994|C0033010|C0034065|C0035078|C0035204|C00 36986|C0038843|C0039070|C0039236|C0080276|C0085207|C0085740|C0152150|C0152151|C0 152152|C0152964|C0155749|C0155774|C0155778|C0155796|C0156373|C0156543|C0156604|C 0156677|C0156699|C0156703|C0156756|C0157535|C0158806|C0158816|C0161754|C0178269| C0178288|C0178298|C0233214|C0242172|C0260550|C0260551|C0263660|C0269294|C026966 1|C0269672|C0271641|C0275667|C0275820|C0302487|C0332544|C0339951|C0340747|C03420 441|C342054|C0342068|C03484471|C348786|C0348859|C0348877|C0348881|C0348950|C0349 253|C0362068|C0391989|C0392070|C0405328|C0411164|C0411169|C0451717|C0451783|C045 1790|C0451791|C0451792|C0451799|C0451800|C0451801|C0451802|C0451803|C0451810|C04 51814|C0451816|C0451822|C0452164|C0452165|C0475523|C0475578|C0476442|C0476491|C0 476550|C0476570|C0476572|C0476635|C0477322|C0477391|C0477762|C0477808|C0477809|C 0477810|C0477811|C0477812|C0477813|C0477814|C0477815|C0477816|C0477817|C0477819| C0477820|C0477865|C0477876|C0477878|C0477881|C0477885|C0478521|C0478618|C049404 8|C0494450|C0494514|C0494598|C0494783|C0495168|C0495169|C0495170|C0495171|C04951 72|C0495173|C0495174|C0495175|C0495176|C0495177|C0495180|C0495184|C0495187|C0495 188|C0495189|C0495190|C0495192|C0495196|C0495197|C0495198|C0495199|C0495224|C049 5225|C0495227|C0495251|C0495292|C0495301|C0495302|C0495304|C0495308|C0495309|C04 95310|C0495311|C0495312|C0495313|C0495314|C0495315|C0495316|C0495317|C0495318|C0 495351|C04 95 6136|C0496158|C0496641|C0496642|C0496643|C 0496644|C0496645|C0496646|C0496693|C0496727|C0694449|C0694451|C0694452|C0694454|C0694456|C0700573|C0728889|C0728936|C0851207|C0851208|C0851209|C0851217|C086921 0|C0869272|C0878544|C1314803|C1367972|C1533618|C1533626|C1704300|C3146283|C36489 68,Brain white matter_STON1-GTF2A1L,1647); (31,C0007871|C0011849|C0011854|C0011860|C0012242|C0012739|C001353 7|C0013927|C0018790|C0018801|C0019693|C0020450|C0020538|C0021361|C0024528|C00274 98|C0027765|C0028962|C0029496|C0029604|C0031154|C0032969|C0032984|C0032987|C0032 989|C0032991|C0032993|C0032994|C0033010|C0034065|C0035078|C0035204|C00369861|C003 8843|C0039070|C0039236|C0080276|C0085207|C0085740|C0152150|C0152151|C0152152|C01 52964|C0155749|C0155774|C0155778|C0155796|C0156373|C0156543|C0156604|C0156677|C0 156699|C0156703|C0156756|C0157535|C0158806|C0158816|C0161754|C0178269|C0178288|C 0178298|C0233214|C0242172|C0260550|C0260551|C0263660|C0269294|C0269661|C0269672| C0271641|C0275667|C0275820|C0302487|C0332544|C0339951|C0340747|C0342044|C034205 4|C0342068|C0348447|C0348786|C0348859|C0348877|C0348881|C0348950|C0349253|C03620 68|C0391989|C0392070|C0405328|C0411164|C0411169|C0451717|C0451783|C0451790|C0451 791|C0451792|C0451799|C0451800|C0451801|C0451802|C0451803|C0451810|C0451814|C045 1816|C0451822|C0452164|C0452165|C0475523|C0475578|C0476442|C0476491|C0476550|C04 76570|C0476572|C0476635|C0477322|C0477391|C0477762|C0477808|C0477809|C0477810|C0 477811|C0477812|C0477813|C0477814|C0477815|C0477816|C0477817|C0477819|C0477820|C 0477865|C0477876|C04778781|C0477881|C0477885|C0478521|C04786181|C0494048|C04944501 89|C0495190|C0495192|C0495196|C0495197|C0495198|C0495199|C0495224|C0495225|C0495 227|C0495251|C0495292|C0495301|C0495302|C0495304|C0495308|C0495309|C0495310|C049 5311|C0495312|C0495313|C0495314|C0495315|C0495316|C0495317|C0495318|C0495351|C04 95699|C0495706|C0496132|C0496136|C0496158|C0496641|C0496642|C0496643|C0496644|C0 496645|C0496646|C0496693|C0496727|C0694449|C0694451|C0694452|C0694454|C0694456|C 0700573|C0728889|C0728936|C0851207|C0851208|C0851209|C0851217|C0869210|C0869272| C0878544|C1314803|C1367972|C1533618|C1533626|C1704300|C3146283|C3648968,Endometr ial tissue_TMEM159,1635); (31,C0007871|C0011849|C0011854|C0011860|C0012242|C0012739|C 0013537|C0013927|C0018790|C0018801|C0019693|C0020450|C0020538|C0021361|C0024528| C0027498|C0027765|C0028962|C0029496|C0029604|C0031154|C0032969|C0032984|C003298 7|C0032989|C0032991|C0032993|C0032994|C0033010|C0034065|C0035078|C0035204|C00369 86|C0038843|C0039070|C0039236|C0080276|C0085207|C0085740|C0152150|C0152151|C0152 152|C0152964|C0155749|C0155774|C0155778|C0155796|C0156373|C0156543|C0156604|C015 6677|C0156699|C0156703|C0156756|C0157535|C0158806|C0158816|C0161754|C0178269|C01 78288|C0178298|C0233214|C0242172|C0260550|C0260551|C0263660|C0269294|C0269661|C0 269672|C0271641|C0275667|C0275820|C0302487|C0332544|C0339951|C0340747|C0342044|C 0342054|C0342068|C0348447|C0348786|C0348859|C0348877|C0348881|C0348950|C0349253| C03620681|C0391989|C0392070|C0405328|C0411164|C0411169|C0451717|C045179 0|C0451791|C0451792|C0451799|C0451800|C0451801|C0451802|C0451803|C0451810|C04518 14|C0451816|C0451822|C0452164|C0452165|C0475523|C0475578|C0476442|C0476491|C0476 550|C0476570|C0476572|C0476635|C0477322|C0477391|C0477762|C0477808|C0477809|C047 7810|C0477811|C0477812|C0477813|C0477814|C0477815|C0477816|C0477817|C0477819|C04 77820|C0477865|C0477876|C0477878|C0477881|C0477885|C0478521|C0478618|C0494048|C0 494450|C0494514|C0494598|C0494783|C0495168|C0495169|C0495170|C0495171|C0495172|C 0495173|C0495174|C0495175|C0495176|C0495177|C0495180|C0495184|C0495187|C0495188| C0495189|C0495190|C0495192|C0495196|C0495197|C0495198|C0495199|C0495224|C049522 5|C0495227|C0495251|C0495292|C0495301|C0495302|C0495304|C0495308|C0495309|C04953 10|C0495311|C0495312|C0495313|C0495314|C0495315|C0495316|C0495317|C0495318|C0495 351|C0495699|C0495706|C0496132|C0496136|C0496158|C0496641|C0496642|C0496643|C049 66441|C0496645|C0496646|C0496693|C0496727|C0694449|C0694451|C0694452|C0694454|C06 94456|C0700573|C0728889|C0728936|C0851207|C0851208|C0851209|C0851217|C0869210|C0 ndometrial tissue_GLB1,1550); (31,C0007871|C0011849|C0011854|C0011860|C0012242|C0012739|C0013 537|C0013927|C0018790|C0018801|C0019693|C0020450|C0020538|C0021361|C0024528|C002 7498|C0027765|C0028962|C0029496|C0029604|C0031154|C0032969|C0032984|C0032987|C00 32989|C0032991|C0032993|C0032994|C0033010|C0034065|C0035078|C0035204|C0036986|C0 038843|C0039070|C0039236|C0080276|C0085207|C0085740|C0152150|C0152151|C0152152|C 0152964|C0155749|C0155774|C0155778|C0155796|C0156373|C0156543|C0156604|C0156677| C0156699|C0156703|C0156756|C0157535|C0158806|C0158816|C0161754|C0178269|C017828 8|C0178298|C0233214|C0242172|C0260550|C0260551|C0263660|C0269294|C0269661|C02696 72|C0271641|C0275667|C0275820|C0302487|C0332544|C0339951|C0340747|C0342044|C0342 054|C0342068|C0348447|C0348786|C0348859|C0348877|C0348881|C0348950|C0349253|C036 2068|C0391989|C0392070|C0405328|C0411164|C0411169|C0451717|C0451783|C0451790|C04 51791|C0451792|C0451799|C0451800|C0451801|C0451802|C0451803|C0451810|C0451814|C0 451816|C0451822|C0452164|C0452165|C0475523|C0475578|C0476442|C0476491|C0476550|C 0476570|C0476572|C0476635|C0477322|C0477391|C0477762|C0477808|C0477809|C0477810| C0477811|C0477812|C0477813|C0477814|C0477815|C0477816|C0477817|C0477819|C047782 0|C0477865|C0477876|C0477878|C0477881|C0477885|C0478521|C0478618|C0494048|C04944 50|C0494514|C0494598|C0494783|C0495168|C0495169|C0495170|C0495171|C0495172|C0495 173|C0495174|C0495175|C0495176|C0495177|C0495180|C0495184|C0495187|C0495188|C049 5189|C0495190|C0495192|C0495196|C0495197|C0495198|C0495199|C0495224|C0495225|C04 95227|C0495251|C0495292|C0495301|C0495302|C0495304|C0495308|C0495309|C0495310|C0 495311|C0495312|C0495313|C0495314|C0495315|C0495316|C0495317|C0495318|C0495351|C 0495699|C0495706|C0496132|C0496136|C0496158|C049664|C0496642|C0496643|C0496644|C0496645|C0496646|C0496693|C0496727|C0694449|C0694451|C0694452|C0694454|C069445 6|C0700573|C0728889|C0728936|C0851207|C0851208|C0851209|C0851217|C0869210|C08692 72|C0878544|C1314803|C1367972|C1533618|C1533626|C1704300|C3146283|C3648968,Mono cyte_PDHX,1494); (31,C0007871|C0011849|C0011854|C0011860|C0012242|C0012739|C00135 37|C0013927|C0018790|C0018801|C0019693|C0020450|C0020538|C0021361|C0024528|C0027 498|C0027765|C0028962|C0029496|C0029604|C0031154|C0032969|C0032984|C0032987|C003 2989|C0032991|C0032993|C0032994|C0033010|C0034065|C0035078|C0035204|C0036986|C00 38843|C0039070|C0039236|C0080276|C0085207|C0085740|C0152150|C0152151|C0152152|C0 152964|C0155749|C0155774|C0155778|C0155796|C0156373|C0156543|C0156604|C0156677|C 0156699|C0156703|C0156756|C0157535|C0158806|C0158816|C0161754|C0178269|C0178288| C0178298|C0233214|C0242172|C0260550|C0260551|C0263660|C0269294|C0269661|C026967 2|C0271641|C0275667|C0275820|C0302487|C0332544|C0339951|C0340747|C0342044|C03420 541|C0342068|C0348447|C03487861|C0348859|C0348877|C0348881|C0348950|C0349253|C0362 068|C0391989|C0392070|C0405328|C0411164|C0411169|C0451717|C0451783|C0451790|C045 1791|C0451792|C0451799|C0451800|C0451801|C0451802|C0451803|C0451810|C0451814|C04 51816|C0451822|C0452164|C0452165|C0475523|C0475578|C0476442|C0476491|C0476550|C0 476570|C0476572|C0476635|C0477322|C0477391|C0477762|C0477808|C0477809|C0477810|C 0477811|C0477812|C0477813|C0477814|C0477815|C0477816|C0477817|C0477819|C0477820| C0477865|C0477876|C0477878|C0477881|C0477885|C0478521|C0478618|C0494048|C049445 0|C0494514|C0494598|C0494783|C0495168|C0495169|C0495170|C0495171|C0495172|C04951 73|C0495174|C0495175|C0495176|C0495177|C0495180|C0495184|C0495187|C0495188|C0495 189|C0495190|C0495192|C0495196|C0495197|C0495198|C0495199|C0495224|C0495225|C049 5227|C0495251|C0495292|C0495301|C0495302|C0495304|C0495308|C0495309|C0495310|C04 95311|C0495312|C0495313|C0495314|C0495315|C0495316|C0495317|C0495318|C0495351|C0 495699|C0495706|C0496132|C0496136|C0496158|C0496641|C0496642|C0496643|C0496644|C 04966451|C0496646|C0496693|C0496727|C0694449|C0694451|C0694452|C0694454|C0694456|C0700573|C0728889|C0728936|C0851207|C0851208|C0851209|C0851217|C0869210|C086927 2|C0878544|C1314803|C1367972|C1533618|C1533626|C1704300|C3146283|C3648968,Bladder_THEM6,1490); (31,C0007871|C0011849|C0011854|C0011860|C0012242|C0012739|C0013537| C0013927|C0018790|C0018801|C0019693|C0020450|C0020538|C0021361|C0024528|C002749 8|C0027765|C0028962|C0029496|C0029604|C0031154|C0032969|C0032984|C0032987|C00329 89|C0032991|C0032993|C0032994|C0033010|C0034065|C0035078|C0035204|C0036986|C0038 843|C0039070|C0039236|C0080276|C0085207|C0085740|C0152150|C0152151|C0152152|C015 2964|C0155749|C0155774|C0155778|C0155796|C0156373|C0156543|C0156604|C0156677|C01 56699|C0156703|C0156756|C0157535|C0158806|C0158816|C0161754|C0178269|C0178288|C0 178298|C0233214|C0242172|C0260550|C026055|C0263660|C0269294|C026966|C0269672|C 0271641|C0275667|C0275820|C0302487|C0332544|C0339951|C0340747|C0342044|C0342054| C0342068|C0348447|C0348786|C0348859|C0348877|C0348881|C0348950|C0349253|C036206 8|C0391989|C0392070|C0405328|C0411164|C0411169|C0451717|C0451783|C0451790|C04517 91|C0451792|C0451799|C0451800|C0451801|C0451802|C0451803|C0451810|C0451814|C0451 816|C0451822|C0452164|C0452165|C0475523|C0475578|C0476442|C0476491|C0476550|C047 6570|C0476572|C0476635|C0477322|C0477391|C0477762|C0477808|C0477809|C0477810|C04 77811|C0477812|C0477813|C0477814|C0477815|C0477816|C0477817|C0477819|C0477820|C0 477865|C0477876|C0477878|C0477881|C0477885|C0478521|C0478618|C0494048|C0494450|C 9|C0495190|C0495192|C0495196|C0495197|C0495198|C0495199|C0495224|C0495225|C04952 27|C0495251|C0495292|C0495301|C0495302|C04953041|C0495308|C0495309|C0495310|C0495 311|C0495312|C0495313|C0495314|C0495315|C0495316|C0495317|C0495318|C0495351|C049 5699|C0495706|C0496132|C0496136|C0496158|C0496641|C0496642|C0496643|C0496644|C04 96645|C0496646|C0496693|C0496727|C0694449|C0694451|C0694452|C0694454|C0694456|C0 700573|C0728889|C0728936|C0851207|C0851208|C0851209|C0851217|C0869210|C0869272|C 0878544|C1314803|C1367972|C1533618|C1533626|C1704300|C3146283|C3648968,Temporal_cortex_MYADML2,1475); (31,C0007871|C0011849|C0011854|C00118601|C0012242|C0012739|C0013537|C0013927|C0018790|C0018801|C0019693|C0020450|C0020538|C0021361|C002452 8|C0027498|C0027765|C0028962|C0029496|C0029604|C0031154|C0032969|C0032984|C00329 87|C0032989|C0032991|C0032993|C0032994|C0033010|C0034065|C0035078|C0035204|C0036 986|C00388431|C0039070|C0039236|C0080276|C0085207|C0085740|C0152150|C0152151|C015 2152|C0152964|C0155749|C0155774|C0155778|C0155796|C0156373|C0156543|C0156604|C01 56677|C0156699|C0156703|C0156756|C0157535|C0158806|C0158816|C0161754|C0178269|C0 178288|C0178298|C0233214|C0242172|C0260550|C0260551|C0263660|C0269294|C0269661|C 0269672|C0271641|C0275667|C0275820|C0302487|C0332544|C0339951|C0340747|C0342044| C03420541|C0342068|C03484471|C0348786|C0348859|C0348877|C0348881|C0348950|C034925 3|C0362068|C0391989|C0392070|C0405328|C0411164|C0411169|C0451717|C0451783|C04517 90|C0451791|C0451792|C0451799|C0451800|C0451801|C0451802|C0451803|C0451810|C0451 814|C0451816|C0451822|C0452164|C0452165|C0475523|C0475578|C0476442|C0476491|C047 6550|C0476570|C0476572|C0476635|C0477322|C0477391|C0477762|C0477808|C0477809|C04 77810|C0477811|C0477812|C0477813|C0477814|C0477815|C0477816|C0477817|C0477819|C0 477820|C0477865|C0477876|C0477878|C0477881|C0477885|C0478521|C0478618|C0494048|C 0494450|C0494514|C0494598|C0494783|C0495168|C0495169|C0495170|C0495171|C0495172| C0495173|C0495174|C0495175|C0495176|C0495177|C0495180|C0495184|C0495187|C049518 8|C0495189|C0495190|C0495192|C0495196|C0495197|C0495198|C0495199|C0495224|C04952 25|C0495227|C0495251|C0495292|C0495301|C0495302|C0495304|C0495308|C0495309|C0495 310|C0495311|C0495312|C0495313|C0495314|C0495315|C0495316|C0495317|C0495318|C049 5351|C0495699|C0495706|C0496132|C0496136|C0496158|C0496641|C0496642|C0496643|C04 96644|C0496645|C0496646|C0496693|C0496727|C0694449|C0694451|C0694452|C0694454|C0 694456|C0700573|C0728889|C0728936|C0851207|C0851208|C0851209|C0851217|C0869210|C 0869272|C0878544|C1314803|C1367972|C1533618|C1533626|C1704300|C3146283|C3648968, Brain white matter_CLEC12A,1473); (31,C00007871|C0011849|C0011854|C0011860|C0012242|C0012739|C 0013537|C0013927|C0018790|C0018801|C0019693|C0020450|C0020538|C0021361|C0024528| C0027498|C0027765|C0028962|C0029496|C0029604|C0031154|C0032969|C0032984|C003298 7|C0032989|C0032991|C0032993|C0032994|C0033010|C0034065|C0035078|C0035204|C00369 86|C0038843|C0039070|C0039236|C0080276|C0085207|C0085740|C0152150|C0152151|C0152 152|C0152964|C0155749|C0155774|C0155778|C0155796|C0156373|C0156543|C0156604|C015 6677|C0156699|C0156703|C0156756|C0157535|C0158806|C0158816|C0161754|C0178269|C01 78288|C0178298|C0233214|C0242172|C0260550|C0260551|C0263660|C0269294|C0269661|C0 269672|C0271641|C0275667|C0275820|C0302487|C0332544|C0339951|C0340747|C0342044|C 0342054|C0342068|C0348447|C0348786|C0348859|C0348877|C0348881|C0348950|C0349253| C03620681|C0391989|C0392070|C0405328|C0411164|C0411169|C0451717|C045179 0|C0451791|C0451792|C0451799|C0451800|C0451801|C0451802|C0451803|C0451810|C04518 14|C0451816|C0451822|C0452164|C0452165|C0475523|C0475578|C0476442|C0476491|C0476 550|C0476570|C0476572|C0476635|C0477322|C0477391|C0477762|C0477808|C0477809|C047 7810|C0477811|C0477812|C0477813|C0477814|C0477815|C0477816|C0477817|C0477819|C04 77820|C0477865|C0477876|C0477878|C0477881|C0477885|C0478521|C0478618|C0494048|C0 494450|C0494514|C0494598|C0494783|C0495168|C0495169|C0495170|C0495171|C0495172|C 0495173|C0495174|C0495175|C0495176|C0495177|C0495180|C0495184|C0495187|C0495188| C0495189|C0495190|C0495192|C0495196|C0495197|C0495198|C0495199|C0495224|C049522 5|C0495227|C0495251|C0495292|C0495301|C0495302|C0495304|C0495308|C0495309|C04953 10|C0495311|C0495312|C0495313|C0495314|C0495315|C0495316|C0495317|C0495318|C0495 351|C0495699|C0495706|C0496132|C0496136|C0496158|C0496641|C0496642|C0496643|C049 66441|C0496645|C0496646|C0496693|C0496727|C0694449|C0694451|C0694452|C0694454|C06 94456|C0700573|C0728889|C0728936|C0851207|C0851208|C0851209|C0851217|C0869210|C0 869272|C0878544|C1314803|C1367972|C1533618|C1533626|C1704300|C3146283|C3648968, Macrophage_MEPE,1424); (31,C000787|C0011849|C0011854|C0011860|C0012242|C0012739|C0013537|C0013927|C0018790|C0018801|C0019693|C0020450|C0020538|C0021361|C002452 8|C0027498|C0027765|C0028962|C0029496|C0029604|C0031154|C0032969|C0032984|C00329 87|C0032989|C0032991|C0032993|C0032994|C0033010|C0034065|C0035078|C0035204|C0036 986|C0038843|C0039070|C00392361|C00802761|C0085207|C0085740|C0152150|C0152151|C015 2152|C0152964|C0155749|C0155774|C0155778|C0155796|C0156373|C0156543|C0156604|C01 56677|C0156699|C0156703|C0156756|C0157535|C0158806|C0158816|C0161754|C0178269|C0 178288|C0178298|C0233214|C0242172|C0260550|C0260551|C0263660|C0269294|C0269661|C 0269672|C0271641|C0275667|C0275820|C0302487|C0332544|C0339951|C0340747|C0342044| C0342054|C0342068|C0348447|C03487861|C0348859|C0348877|C0348881|C0348950|C034925 3|C0362068|C0391989|C0392070|C0405328|C0411164|C0411169|C0451717|C0451783|C04517 90|C0451791|C0451792|C0451799|C0451800|C0451801|C0451802|C0451803|C0451810|C0451 814|C0451816|C0451822|C0452164|C0452165|C0475523|C0475578|C0476442|C0476491|C047 6550|C0476570|C0476572|C0476635|C0477322|C0477391|C0477762|C0477808|C0477809|C04 77810|C0477811|C0477812|C0477813|C0477814|C0477815|C0477816|C0477817|C0477819|C0 477820|C0477865|C0477876|C0477878|C0477881|C0477885|C0478521|C0478618|C0494048|C 0494450|C0494514|C0494598|C0494783|C0495168|C0495169|C0495170|C0495171|C0495172| C0495173|C0495174|C0495175|C0495176|C0495177|C0495180|C0495184|C0495187|C049518 8|C0495189|C0495190|C0495192|C0495196|C0495197|C0495198|C0495199|C0495224|C04952 25|C0495227|C0495251|C0495292|C0495301|C0495302|C0495304|C0495308|C0495309|C0495 3101|C0495311|C0495312|C0495313|C04953141|C0495315|C0495316|C0495317|C0495318|C049 5351|C0495699|C0495706|C0496132|C0496136|C0496158|C0496641|C0496642|C0496643|C04 96644|C0496645|C0496646|C0496693|C0496727|C0694449|C0694451|C0694452|C0694454|C0 694456|C0700573|C0728889|C0728936|C0851207|C0851208|C0851209|C0851217|C0869210|C 0869272|C0878544|C1314803|C1367972|C1533618|C1533626|C1704300|C3146283|C3648968, Endometrial tissue_PODN,1416); (32,C0008312,Whole_blood_AKIRIN2,2911); (32,C0008312,Whole_blood_AKIRIN2,2911); (32,C0008312,Whole_blood_AKIRIN2,2911); (32,C0008312,Monocyte_PER 2,2255); (32,C0008312,Monocyte_PER2,2255); (32,C0008312,Monocyte_PER2,2255); (32,C000 8312,Colon_RAMP2,2035); (32,C0008312,Colon_RAMP2,2035); (32,C0008312,Colon_RAMP2 ,2035); (32,C0008312,Unknown_CD86,1905); (32,C0008312,Unknown_CD86,1905); (32,C0008 312,Unknown_CD86,1905); (32,C0008312,Monocyte_RILP,1893); (32,C0008312,Monocyte_RI LP,1893); (32,C0008312,Monocyte_RILP,1893); (32,C0008312,Monocyte_SNX9,1875); (32,C00 08312,Monocyte_SNX9,1875); (32,C0008312,Monocyte_SNX9,1875); (32,C0008312,Endometri al tissue_TVP23CP2,1868); (32,C0008312,Endometrial tissue_TVP23CP2,1868); (33,C0008677|C0024117|C0264222|C0348686|C0348693|C0348817|C 03488181|C0494659,Whole_blood_AKIRIN2,2911); (33,C00086771|C00241171|C0264222|C03486 86|C0348693|C0348817|C0348818|C0494659,Monocyte_PER2,2255); (33,C0008677|C0024117| C0264222|C0348686|C0348693|C0348817|C0348818|C0494659,Colon_RAMP2,2035); (33,C00 08677|C0024117|C0264222|C0348686|C0348693|C0348817|C0348818|C0494659,Unknown_C D86,1905); (33,C0008677|C0024117|C0264222|C0348686|C0348693|C0348817|C0348818|C049 4659,Monocyte_RILP,1893); (33,C0008677|C0024117|C0264222|C0348686|C0348693|C034881 7|C0348818|C0494659,Monocyte_SNX9,1875); (33,C0008677|C0024117|C0264222|C0348686|C0348693|C0348817|C0348818|C0494659,Endometrial tissue_TVP23CP2,1868); (33,C0008677|C0024117|C0264222|C0348686|C0348693|C0348817|C 0348818|C0494659,Monocyte_EIF3G,1705); (33,C0008677|C0024117|C0264222|C0348686|C03 48693|C0348817|C0348818|C0494659,Endometrial tissue_SMC2,1699); (33,C0008677|C0024117|C0264222|C0348686|C0348693|C0348817|C0348 818|C0494659,Temporal_cortex_TRIM37,1687); (33,C0008677|C0024117|C0264222|C0348686|C0348693|C03488171|C03488181|C0494659,Colorectum_VPS37B,1656); (33,C0008677|C002411 7|C0264222|C0348686|C0348693|C0348817|C0348818|C0494659,Brain white matter_STON1-GTF2A1L,1647); (33,C0008677|C0024117|C0264222|C0348686|C0348693|C0348817|C034881 8|C0494659,Endometrial tissue_TMEM159,1635); (33,C0008677|C0024117|C0264222|C0348686|C0348693|C0348817|C 03488181|C0494659,Endometrial tissue_GLB1,1550); (33,C0008677|C0024117|C0264222|C0348686|C0348693|C0348817|C0348 818|C0494659,Monocyte_PDHX,1494); (33,C0008677|C0024117|C0264222|C0348686|C034869 3|C0348817|C0348818|C0494659,Bladder_THEM6,1490); (33,C0008677|C0024117|C0264222| C0348686|C0348693|C03488171|C03488181|C0494659,Temporal_cortex_MYADML2,1475); (33, C0008677|C0024117|C0264222|C0348686|C0348693|C0348817|C0348818|C0494659,Brain white matter_CLEC12A,1473); (33,C0008677|C0024117|C0264222|C0348686|C0348693|C0348817|C 0348818|C0494659,Macrophage_MEPE,1424); (33,C0008677|C0024117|C0264222|C0348686|C 0348693|C0348817|C0348818|C0494659,Endometrial tissue_PODN,1416); (34,C0008684|C0155937|C0348160|C0494704|C0494706,Whole_blood_A KIRIN2,2911); (34,C0008684|C0155937|C0348160|C0494704|C0494706,Monocyte_PER2,2255); (34,C0008684|C0155937|C0348160|C0494704|C0494706,Colon_RAMP2,2035); (34,C0008684|C0155937|C0348160|C0494704|C0494706,Unknown_CD86,1905); (34,C0008684|C0155937|C0 348160|C0494704|C0494706,Monocyte_RILP,1893); (34,C0008684|C0155937|C0348160|C0494 704|C0494706,Monocyte_SNX9,1875); (34,C0008684|C0155937|C0348160|C0494704|C049470 6,Endometrial tissue_TVP23CP2,1868); (34,C0008684|C0155937|C0348160|C0494704|C0494706,Monocyte_E IF3G,1705); (34,C0008684|C0155937|C0348160|C0494704|C0494706,Endometrial tissue_SMC2,1699); (34,C0008684|C0155937|C0348160|C0494704|C0494706,Temporal_cortex TRIM37,1687); (34,C0008684|C0155937|C0348160|C0494704|C0494706,Colorectum_VPS37 B,1656); (34,C00086841|C0155937|C03481601|C04947041|C0494706,Brain white matter_STON1-GTF2A1L,1647); (34,C0008684|C0155937|C03481601|C0494704|C0494706,Endometrial tissue_TMEM159,1635); (34,C0008684|C0155937|C0348160|C0494704|C0494706,Endometrial tissue_GLB1,1550); (34,C0008684|C0155937|C0348160|C0494704|C0494706,Monocyte_PDHX ,1494); (34,C00086841|C0155937|C03481601|C04947041|C0494706,Bladder_THEM6,1490); (34,C0 008684|C0155937|C03481601|C0494704|C0494706,Temporal_cortex_MYADML2,1475); (34,C0 0086841|C01559371|C03481601|C04947041|C0494706,Brain white matter_CLEC12A,1473); (34,C0008684|C0155937|C0348160|C0494704|C0494706,Macrophage_MEPE,1424); (34,C0008684|C0155937|C03481601|C0494704|C0494706,Endometrial tissue_PODN,1416); (36,C00008924|C0008925|C0158646|C0432090|C0432098|C0451896|C0451 897|C0477977|C0478022|C0495504|C0495539|C0495540|C0495541|C0495542|C0495543|C049 5545|C0869081|C2981150,Whole_blood_AKIRIN2,2911); (36,C00008924|C0008925|C0158646| C0432090|C0432098|C0451896|C0451897|C0477977|C04780221|C0495504|C04955391|C049554 O|C0495541|C0495542|C0495543|C0495545|C0869081|C2981150,Monocyte_PER2,2255); (36,C 0008924|C0008925|C0158646|C0432090|C0432098|C0451896|C0451897|C0477977|C0478022| C0495504|C0495539|C0495540|C0495541|C0495542|C0495543|C0495545|C0869081|C298115 0,Colon_RAMP2,2035); (36,C0008924|C0008925|C0158646|C0432090|C0432098|C0451896|C0 451897|C0477977|C0478022|C0495504|C0495539|C0495540|C0495541|C0495542|C0495543|C 0495545|C0869081|C2981150,Unknown_CD86,1905); (36,C0008924|C0008925|C0158646|C04 32090|C0432098|C0451896|C0451897|C0477977|C0478022|C0495504|C0495539|C04955401|C0 49554|C0495542|C0495543|C0495545|C086908|C2981150,Monocyte_RILP,1893); (36,C0008 924|10008925|C0158646|C0432090|C0432098|C0451896|C0451897|C0477977|C0478022|C049 5504|C0495539|C0495540|C0495541|C0495542|C0495543|C0495545|C0869081|C2981150,Mo nocyte_SNX9,1875); (36,C0008924|C0008925|C0158646|C0432090|C0432098|C0451896|C045 1897|C0477977|C0478022|C0495504|C0495539|C0495540|C0495541|C0495542|C0495543|C04 95545|C0869081|C2981150,Endometrial tissue_TVP23CP2,1868); (36,C0008924|C0008925|C0158646|C0432090|C0432098|C0451896|C 0451897|C0477977|C0478022|C0495504|C0495539|C0495540|C0495541|C0495542|C0495543| C0495545|C0869081|C2981150,Monocyte_EIF3G,1705); (36,C00008924|C0008925|C0158646|C 0432090|C0432098|C0451896|C0451897|C0477977|C0478022|C0495504|C0495539|C0495540| C0495541|C0495542|C0495543|C0495545|C0869081|C2981150,Endometrial tissue_SMC2,1699); (36,C0008924|C0008925|C0158646|C0432090|C0432098|C0451896|C0451 897|C0477977|C0478022|C0495504|C0495539|C0495540|C0495541|C0495542|C0495543|C049 5545|C0869081|C2981150,Temporal_cortex_TRIM37,1687); (36,C00089241|C0008925|C015864 6|C0432090|C0432098|C0451896|C0451897|C0477977|C0478022|C0495504|C04955391|C04955 40|C0495541|C0495542|C0495543|C0495545|C0869081|C2981150,Colorectum_VPS37B,1656) ; (36,C0008924|C0008925|C0158646|C0432090|C0432098|C0451896|C0451897|C0477977|C047 8022|C0495504|C0495539|C0495540|C0495541|C0495542|C0495543|C0495545|C0869081|C29 81150,Brain white matter_STON1-GTF2A1L,1647); (36,C0008924|C0008925|C0158646|C0432090|C0432098|C0451896|C045189 7|C0477977|C0478022|C0495504|C0495539|C0495540|C0495541|C0495542|C0495543|C04955 45|C0869081|C2981150,Endometrial tissue_TMEM159,1635); (36,C0008924|C0008925|C0158646|C0432090|C0432098|C0451896|C 0451897|C0477977|C0478022|C0495504|C0495539|C0495540|C0495541|C0495542|C0495543| C0495545|C0869081|C2981150,Endometrial tissue_GLB1,1550); (36,C0008924|C0008925|C0158646|C0432090|C0432098|C0451896|C0451 897|C0477977|C0478022|C0495504|C0495539|C0495540|C0495541|C0495542|C0495543|C049 5545|C0869081|C2981150,Monocyte_PDHX,1494); (36,C0008924|C0008925|C0158646|C04320 90|C0432098|C0451896|C0451897|C0477977|C0478022|C0495504|C0495539|C0495540|C0495 541|C0495542|C0495543|C0495545|C0869081|C2981150,Bladder_THEM6,1490); (36,C000892 4|C0008925|C0158646|C0432090|C0432098|C0451896|C0451897|C0477977|C0478022|C04955 04|C0495539|C0495540|C0495541|C0495542|C0495543|C0495545|C0869081|C2981150,Temp oral_cortex_MYADML2,1475); (36,C0008924|C0008925|C0158646|C0432090|C0432098|C045 1896|C0451897|C0477977|C0478022|C0495504|C0495539|C0495540|C0495541|C0495542|C04 95543|C0495545|C0869081|C2981150,Brain white matter_CLEC12A,1473); (36,C0008924|C0008925|C0158646|C0432090|C0432098|C0451896|C 0451897|C0477977|C0478022|C0495504|C0495539|C0495540|C0495541|C0495542|C0495543| C0495545|C086908|C2981150,Macrophage_MEPE,1424); (36,C0008924|C0008925|C0158646|C0432090|C0432098|C0451896|C0451897|C0477977|C0478022|C0495504|C0495539|C049554 0|C0495541|C0495542|C0495543|C0495545|C0869081|C2981150,Endometrial tissue_PODN,1416); (40,C0009806|C00140891|C0278008,Whole_blood_AKIRIN2,2911); (40,C0 009806|C0014089|C0278008,Monocyte_PER2,2255); (40,C0009806|C0014089|C0278008,Colon_RAMP2,2035); (40,C0009806|C0014089|C0278008,Unknown_CD86,1905); (40,C0009806|C00 140891|C0278008,Monocyte_RILP,1893); (40,C0009806|C0014089|C0278008,Monocyte_SNX9, 1875); (40,C0009806|C00140891|C0278008,Endometrial tissue_TVP23CP2,1868); (40,C0009806|C0014089|C0278008,Monocyte_EIF3G,1705); (40,C000 9806|C00140891|C0278008,Endometrial tissue_SMC2,1699); (40,C0009806|C00140891|C0278008,Temporal_cortex_TRIM37,1687); (40, C0009806|C00140891|C0278008,Colorectum_VPS37B,1656); (40,C0009806|C00140891|C027800 8,Brain white matter_STON1-GTF2A1L,1647); (40,C0009806|C00140891|C0278008,Endometrial tissue_TMEM159,1635); (40,C0009806|C00140891|C0278008,Endometrial tissue_GLB1,1550); (40,C0009806|C00140891|C0278008,Monocyte PDHX,1494); (40,C0009806|C0014089|C0278008,Bladder_THEM6,1490); (40,C0009806|C0014089|C0278008,Temporal_co rtex_MYADML2,1475); (40,C0009806|C00140891|C0278008,Brain white matter_CLEC12A,1473); (40,C0009806|C0014089|C0278008,Macrophage_MEPE,1424); (40,C0 009806|C0014089|C0278008,Endometrial tissue_PODN,1416); (41,C0010054,Whole_blood_AKIRIN2,2911); (41,C0010054,Monocyte_PE R2,2255); (41,C0010054,Colon_RAMP2,2035); (41,C0010054,Unknown_CD86,1905); (41,C001 0054,Monocyte_RILP,1893); (41,C0010054,Monocyte_SNX9,1875); (41,C0010054,Endometrial tissue_TVP23CP2,1868); (41,C0010054,Monocyte_EIF3G,1705); (41,C0010054,Endometrial tissue_SMC2,1699); (41,C0010054,Temporal_cortex_TRIM37,1687); (41,C0010054,Colorectum VPS37B,1656); (41,C0010054,Brain white matter_STON1-GTF2A1L,1647); (41,C0010054,Endometrial tissue_TMEM159,1635); (41,C0010054,Endometrial tissue_GLB1,1550); (41,C0010054,Monocyte_PDHX,1494); (41,C0010054,Bladder_THEM6,14 90); (41,C0010054,Temporal_cortex_MYADML2,1475); (41,C0010054,Brain white matter_CLEC12A,1473); (41,C0010054,Macrophage_MEPE,1424); (41,C0010054,Endometrial tissue_PODN,1416); (42,C00100541|C0348590,Whole_blood_AKIRIN2,2911); (42,C00100541|C0 348590,Monocyte_PER2,2255); (42,C00100541|C0348590,Colon_RAMP2,2035); (42,C0010054|C0348590,Unknown_CD86,1905); (42,C0010054|C0348590,Monocyte_RILP,1893); (42,C00100 54|C0348590,Monocyte_SNX9,1875); (42,C0010054|C0348590,Endometrial tissue_TVP23CP2,1868); (42,C00100541|C0348590,Monocyte_EIF3G,1705); (42,C00100541|C034 8590,Endometrial tissue_SMC2,1699); (42,C00100541|C0348590,Temporal_cortex_TRIM37,1687); (42,C0010054|C0348590,Colorectum_VPS37B,1656); (42,C00100541|C0348590,Brain white matter_STON1-GTF2A1L,1647); (42,C0010054|C0348590,Endometrial tissue_TMEM159,1635); (42,C0010054|C0348590,Endometrial tissue_GLB1,1550); (42,C0010054|C0348590,Monocyte_PDHX,1494); (42,C0010054|C0348590,Bladder_THEM6,1490); (42,C0010054|C0348590,Temporal_cortex_MYADML2,1475); (42,C0 0100541|C0348590,Brain white matter_CLEC12A,1473); (42,C0010054|C0348590,Macrophage_MEPE,1424); (42,C0010054|C0 348590,Endometrial tissue_PODN,1416); (43,C0010346|C0156146|C0156147|C0348736|C0409695|C0451836,Whole _blood_AKIRIN2,2911); (43,CUU1U346|CU16146|C13614,MC348/36|CU4U9693|CU4M1836,M onocyte_PER2,2255); (43,C0010346|C0156146|C0156147|C0348736|C0409695|C0451836,Colo n_RAMP2,2035); (43,C0010346|C0156146|C0156147|C0348736|C0409695|C0451836,Unknown_CD86,1905); (43,C0010346|C0156146|C0156147|C0348736|C0409695|C0451836,Monocyte_R ILP,1893); (43,C0010346|C0156146|C0156147|C0348736|C0409695|C0451836,Monocyte_SNX 9,1875); (43,C0010346|C0156146|C0156147|C0348736|C0409695|C0451836,Endometrial tissue_TVP23CP2,1868); (43,C0010346|C0156146|C0156147|C03487361|C0409695|C0451836,M onocyte_EIF3G,1705); (43,C0010346|C0156146|C0156147|C0348736|C0409695|C0451836,End ometrial tissue_SMC2,1699); (43,C0010346|C0156146|C0156147|C0348736|C0409695|C0451836,Tempo ral_cortex_TRIM37,1687); (43,C0010346|C0156146|C0156147|C0348736|C0409695|C0451836, Colorectum_VPS37B,1656); (43,C0010346|C0156146|C0156147|C0348736|C0409695|C045183 6,Brain white matter_STON1-GTF2A1L,1647); (43,C0010346|C0156146|C0156147|C0348736|C0409695|C0451836,Endometr ial tissue_TMEM159,1635); (43,C0010346|C0156146|C0156147|C0348736|C0409695|C0451836,E ndometrial tissue_GLB1,1550); (43,C0010346|C0156146|C0156147|C0348736|C0409695|C0451836,Monoc yte_PDHX,1494); (43,C00010346|C0156146|C0156147|C0348736|C0409695|C0451836,Bladder_THEM6,1490); (43,C0010346|C0156146|C0156147|C0348736|C0409695|C0451836,Temporal_c ortex_MYADML2,1475); (43,C00010346|C0156146|C0156147|C0348736|C0409695|C0451836,B rain white matter_CLEC12A,1473); (43,C0010346|C0156146|C0156147|C0348736|C0409695|C0451836,M acrophage_MEPE,1424); (43,C0010346|C0156146|C0156147|C0348736|C0409695|C0451836,E ndometrial tissue_PODN,1416); (51,C0011849|C0011854|C0011860|C0271641|C0348447|C0851207,Whole_blood_AKIRIN2,2911); (51,C0011849|C0011854|C0011860|C0271641|C0348447|C0851207,W hole_blood_AKIRIN2,2911); (51,C0011849|C0011854|C0011860|C0271641|C0348447|C085120 7,Monocyte_PER2,2255); (51,C0011849|C0011854|C0011860|C0271641|C0348447|C0851207, Monocyte_PER2,2255); (51,C0011849|C0011854|C0011860|C0271641|C0348447|C0851207,C0 lon_RAMP2,2035); (51,C00011849|C0011854|C0011860|C0271641|C0348447|C0851207,Colon_RAMP2,2035); (51,C00011849|C0011854|C0011860|C0271641|C0348447|C0851207,Unknown_CD86,1905); (51,C0011849|C0011854|C0011860|C0271641|C0348447|C0851207,Unknown_CD 86,1905); (51,C0011849|C0011854|C0011860|C0271641|C0348447|C0851207,Monocyte_RILP, 1893); (51,C0011849|C0011854|C0011860|C0271641|C0348447|C0851207,Monocyte_RILP,189 3); (51,C0011849|C0011854|C0011860|C0271641|C0348447|C0851207,Monocyte_SNX9,1875); (51,C0011849|C0011854|C0011860|C0271641|C0348447|C0851207,Monocyte_SNX9,1875); (5 1,C0011849|C0011854|C0011860|C0271641|C0348447|C0851207,Endometrial tissue_TVP23CP2,1868); (51,C0011849|C0011854|C0011860|C0271641|C0348447|C0851207,E ndometrial tissue_TVP23CP2,1868); (51,C0011849|C0011854|C0011860|C0271641|C0348447|C0851207,M onocyte_EIF3G,1705); (51,C0011849|C0011854|C0011860|C0271641|C0348447|C0851207,Mon ocyte_EIF3G,1705); (51,C0011849|C0011854|C0011860|C0271641|C0348447|C0851207,Endom etrial tissue_SMC2,1699); (51,C0011849|C0011854|C0011860|C0271641|C0348447|C0851207,Endom etrial tissue_SMC2,1699); (51,C0011849|C0011854|C0011860|C0271641|C0348447|C0851207,Tempo ral_cortex_TRIM37,1687); (51,C0011849|C0011854|C00118601|C0271641|C0348447|C0851207, Temporal_cortex_TRIM37,1687); (56,C0013395|C00188341|C0232492,Whole_blood_AKIRIN2, 2911); (56,C0013395|C00188341|C0232492,Monocyte_PER2,2255); (56,C0013395|C00188341|C0 232492,Colon_RAMP2,2035); (56,C0013395|C0018834|C0232492,Unknown_CD86,1905); (56, C0013395|C0018834|C0232492,Monocyte_RILP,1893); (56,C0013395|C0018834|C0232492,Mo nocyte_SNX9,1875); (56,C00013395|C0018834|C0232492,Endometrial tissue_TVP23CP2,1868); (56,C0013395|C0018834|C0232492,Monocyte_EIF3G,1705); (56,C001 3395|C00188341|C0232492,Endometrial tissue_SMC2,1699); (56,C0013395|C00188341|C0232492,Temporal_cortex_TRIM37,1687); (56, C0013395|C00188341|C0232492,Colorectum_VPS37B,1656); (56,C0013395|C00188341|C023249 2,Brain white matter_STON1-GTF2A1L,1647); (56,C0013395|C00188341|C0232492,Endometrial tissue_TMEM159,1635); (56,C0013395|C00188341|C0232492,Endometrial tissue_GLB1,1550); (56,C0013395|C00188341|C0232492,Monocyte_PDHX,1494); (56,C0013395|C0018834|C0232492,Bladder_THEM6,1490); (56,C0013395|C0018834|C0232492,Temporal_co rtex_MYADML2,1475); (56,C0013395|C00188341|C0232492,Brain white matter_CLEC12A,1473); (56,C0013395|C0018834|C0232492,Macrophage_MEPE,1424); (56,C0 013395|C00188341|C0232492,Endometrial tissue_PODN,1416); (62,C0015674|C0024528|C0027804|C0152145|C0236795|C0236816|C0451 880|C0477817|C0546983|C1270972,Whole_blood_AKIRIN2,2911); (62,C0015674|C0024528|C 0027804|C0152145|C0236795|C0236816|C0451880|C0477817|C0546983|C1270972,Monocyte_PER2,2255); (62,C00156741|C0024528|C0027804|C0152145|C0236795|C0236816|C0451880|C0 45|C0236795|C0236816|C0451880|C0477817|C0546983|C1270972,Unknown_CD86,1905); (62, C0015674|C0024528|C0027804|C0152145|C0236795|C0236816|C0451880|C0477817|C054698 3|C1270972,Monocyte_RILP,1893); (62,C0015674|C0024528|C0027804|C0152145|C0236795|C 0236816|C0451880|C0477817|C0546983|C1270972,Monocyte_SNX9,1875); (62,C0015674|C00 24528|C0027804|C0152145|C0236795|C0236816|C0451880|C0477817|C0546983|C1270972,En dometrial tissue_TVP23CP2,1868); (62,C0015674|C0024528|C0027804|C0152145|C0236795|C0236816|C 0451880|C0477817|C0546983|C1270972,Monocyte_EIF3G,1705); (62,C0015674|C0024528|C00 27804|C0152145|C0236795|C0236816|C0451880|C0477817|C0546983|C1270972,Endometrial tissue_SMC2,1699); (62,C0015674|C0024528|C0027804|C0152145|C0236795|C0236816|C0451 880|C0477817|C0546983|C1270972,Temporal_cortex_TRIM37,1687); (62,C0015674|C0024528|C0027804|C0152145|C0236795|C0236816|C0451880|C0477817|C0546983|C1270972,Colorectu m_VPS37B,1656); (62,C0015674|C0024528|C0027804|C0152145|C0236795|C0236816|C04518 801|C0477817|C0546983|C1270972,Brain white matter_STON1-GTF2A1L,1647); (62,C00156741|C0024528|C0027804|C0152145|C0236795|C0236816|C045188 0|C04778171|C0546983|C1270972,Endometrial tissue_TMEM159,1635); (62,C0015674|C0024528|C0027804|C0152145|C0236795|C0236816|C 04518801|C04778171|C0546983|C1270972,Endometrial tissue_GLB1,1550); (62,C0015674|C0024528|C0027804|C0152145|C0236795|C0236816|C0451 880|C0477817|C0546983|C1270972,Monocyte_PDHX,1494); (62,C0015674|C0024528|C002780 4|C0152145|C0236795|C0236816|C0451880|C0477817|C0546983|C1270972,Bladder_THEM6, 1490); (62,C0015674|C0024528|C0027804|C0152145|C0236795|C0236816|C0451880|C0477817|C0546983|C1270972,Temporal_cortex_MYADML2,1475); (62,C00156741|C0024528|C0027804| C0152145|C0236795|C0236816|C0451880|C0477817|C0546983|C1270972,Brain white matter_CLEC12A,1473); (62,C0015674|C0024528|C0027804|C0152145|C0236795|C0236816|C 0451880|C0477817|C0546983|C1270972,Macrophage_MEPE,1424); (62,C00156741|C0024528|C 0027804|C0152145|C0236795|C0236816|C0451880|C0477817|C0546983|C1270972,Endometri al tissue_PODN,1416); (64,C0017154,Whole_blood_AKIRIN2,2911); (64,C0017154,Whole_blood_AKIRIN2,2911); (64,C0017154,Whole_blood_AKIRIN2,2911); (64,C0017154,Monocyte PER 2,2255); (64,C0017154,Monocyte_PER2,2255); (64,C0017154,Monocyte PER2,2255); (64,C001 7154,Colon_RAMP2,2035); (64,C0017154,Colon_RAMP2,2035); (64,C0017154,Colon_RAMP2 ,2035); (64,C0017154,Unknown_CD86,1905); (64,C0017154,Unknown_CD86,1905); (64,C0017 154,Unknown_CD86,1905); (64,C0017154,Monocyte_RILP,1893); (64,C0017154,Monocyte_RI LP,1893); (64,C0017154,Monocyte_RILP,1893); (64,C0017154,Monocyte_SNX9,1875); (64,C00 17154,Monocyte_SNX9,1875); (64,C0017154,Monocyte_SNX9,1875); (64,C0017154,Endometri al tissue_TVP23CP2,1868); (64,C0017154,Endometrial tissue_TVP23CP2,1868); (68,C0018681|C0149931|C0338480|C0338483|C0477373|C1735856, Whole_blood_AKIRIN2,2911); (68,C0018681|C0149931|C0338480|C0338483|C0477373|C1735 856,Monocyte_PER2,2255); (68,C0018681|C0149931|C0338480|C0338483|C0477373|C173585 6,Colon_RAMP2,2035); (68,C0018681|C0149931|C0338480|C0338483|C0477373|C1735856,Un known_CD86,1905); (68,C0018681|C0149931|C0338480|C0338483|C0477373|C1735856,Mono cyte_RILP,1893); (68,C0018681|C0149931|C0338480|C0338483|C0477373|C1735856,Monocyt e_SNX9,1875); (68,C0018681|C0149931|C0338480|C0338483|C0477373|C1735856,Endometria 1 tissue_TVP23CP2,1868); (68,C0018681|C0149931|C0338480|C0338483|C0477373|C1735856,M onocyte_EIF3G,1705); (68,C0018681|C0149931|C0338480|C0338483|C0477373|C1735856,End ometrial tissue_SMC2,1699); (68,C0018681|C0149931|C0338480|C0338483|C0477373|C1735856,Tempo ral_cortex_TRIM37,1687); (68,C0018681|C0149931|C0338480|C0338483|C0477373|C1735856, Colorectum_VPS37B,1656); (68,C0018681|C0149931|C0338480|C0338483|C0477373|C173585 6,Brain white matter_STON1-GTF2A1L,1647); (68,C0018681|C0149931|C0338480|C0338483|C0477373|C1735856,Endometr ial tissue_TMEM159,1635); (68,C0018681|C0149931|C0338480|C0338483|C0477373|C1735856,E ndometrial tissue_GLB1,1550); (68,C0018681|C0149931|C0338480|C0338483|C0477373|C1735856,Monoc yte_PDHX,1494); (68,C0018681|C0149931|C0338480|C0338483|C0477373|C1735856,Bladder_THEM6,1490); (68,C0018681|C0149931|C0338480|C0338483|C0477373|C1735856,Temporal_c ortex_MYADML2,1475); (68,C0018681|C0149931|C0338480|C0338483|C0477373|C1735856,B rain white matter_CLEC12A,1473); (68,C0018681|C0149931|C0338480|C0338483|C0477373|C1735856,M acrophage_MEPE,1424); (68,C0018681|C0149931|C0338480|C0338483|C0477373|C1735856,E ndometrial tissue_PODN,1416); (73,C00193641|C0022568|C00022573|C0041322|C0155087|C0339239|C0348 191|C0348526|C0348532|C0348533|C0348985|C0494081|C0494524,Whole_blood_AKIRIN2,2 911); (73,C0019364|C0022568|C0022573|C0041322|C0155087|C0339239|C0348191|C0348526| C0348532|C0348533|C0348985|C0494081|C0494524,Monocyte_PER2,2255); (73,C0019364|C0 022568|C0022573|C0041322|C0155087|C0339239|C0348191|C0348526|C0348532|C0348533|C 0348985|C0494081|C0494524,Colon_RAMP2,2035); (73,C0019364|C0022568|C0022573|C0041 322|C0155087|C0339239|C0348191|C0348526|C0348532|C0348533|C0348985|C0494081|C049 4524,Unknown_CD86,1905); (73,C0019364|C0022568|C0022573|C0041322|C0155087|C03392 39|C0348191|C0348526|C0348532|C0348533|C0348985|C0494081|C0494524,Monocyte_RILP, 1893); (73,C0019364|C0022568|C0022573|C0041322|C0155087|C0339239|C0348191|C0348526|C0348532|C0348533|C0348985|C0494081|C0494524,Monocyte_SNX9,1875); (73,C0019364|C 0022568|C0022573|C0041322|C0155087|C0339239|C0348191|C0348526|C0348532|C0348533| C0348985|C0494081|C0494524,Endometrial tissue_TVP23CP2,1868); (73,C0019364|C0022568|C0022573|C0041322|C0155087|C0339239|C 0348191|C0348526|C0348532|C0348533|C0348985|C0494081|C0494524,Monocyte_EIF3G,17 05); (73,C0019364|C0022568|C0022573|C0041322|C0155087|C0339239|C0348191|C0348526|C 0348532|C0348533|C0348985|C0494081|C0494524,Endometrial tissue_SMC2,1699); (73,C0019364|C0022568|C0022573|C0041322|C0155087|C0339239|C0348 191|C0348526|C0348532|C0348533|C0348985|C0494081|C0494524,Temporal_cortex_TRIM37 ,1687); (73,C0019364|C0022568|C0022573|C0041322|C0155087|C0339239|C0348191|C034852 6|C0348532|C0348533|C0348985|C0494081|C0494524,Colorectum_VPS37B,1656); (73,C00193 64|C0022568|C0022573|C0041322|C0155087|C0339239|C0348191|C0348526|C0348532|C0348 533|C0348985|C0494081|C0494524,Brain white matter_STON1-GTF2A1L,1647); (73,C0019364|C0022568|C0022573|C0041322|C0155087|C0339239|C034819 1|C0348526|C0348532|C0348533|C0348985|C0494081|C0494524,Endometrial tissue_TMEM159,1635); (73,C0019364|C0022568|C0022573|C0041322|C0155087|C0339239|C 0348191|C0348526|C0348532|C0348533|C0348985|C0494081|C0494524,Endometrial tissue_GLB1,1550); (73,C0019364|C0022568|C0022573|C0041322|C0155087|C0339239|C0348 191|C0348526|C0348532|C0348533|C0348985|C0494081|C0494524,Monocyte_PDHX,1494); (7 3,C0019364|C0022568|C0022573|C0041322|C0155087|C0339239|C0348191|C0348526|C03485 32|C0348533|C0348985|C0494081|C0494524,Bladder_THEM6,1490); (73,C0019364|C0022568|C0022573|C0041322|C0155087|C0339239|C0348191|C0348526|C0348532|C0348533|C034898 5|C049408|C0494524,Temporal_cortex_MYADML2,1475); (73,C0019364|C0022568|C002257 3|C0041322|C0155087|C0339239|C0348191|C0348526|C0348532|C0348533|C0348985|C04940 81|C0494524,Brain white matter_CLEC12A,1473); (73,C0019364|C0022568|C0022573|C0041322|C0155087|C0339239|C 0348191|C0348526|C0348532|C0348533|C0348985|C0494081|C0494524,Macrophage_MEPE,1 424); (73,C0019364|C0022568|C0022573|C0041322|C0155087|C0339239|C0348191|C0348526| C0348532|C0348533|C0348985|C0494081|C0494524,Endometrial tissue_PODN,1416); (74,C0019693,Whole_blood_AKIRIN2,2911); (74,C0019693,Whole_blood_AKIRIN2,2911); (74,C0019693,Monocyte_PER2,2255); (74,C0019693,Monocyte_PER2,2255); (74,C0019693,Colon_RAMP2,2035); (74,C0019693,Colon_RAMP2,2035); (74,C0019693,Unkn own_CD86,1905); (74,C0019693,Unknown_CD86,1905); (74,C0019693,Monocyte_RILP,1893); (74,C0019693,Monocyte_RILP,1893); (74,C00019693,Monocyte_SNX9,1875); (74,C0019693,Mo nocyte_SNX9,1875); (74,C0019693,Endometrial tissue_TVP23CP2,1868); (74,C0019693,Endometrial tissue_TVP23CP2,1868); (74,C0019693,Monocyte_EIF3G,1705); (74,C0019693,Monocyte_EIF3 G,1705); (74,C0019693,Endometrial tissue_SMC2,1699); (74,C0019693,Endometrial tissue_SMC2,1699); (74,C00019693,Temporal_cortex_TRIM37,1687); (74,C0019693,Temporal_c ortex_TRIM37,1687); (75,C00201791|C0349080,Whole_blood_AKIRIN2,2911); (75,C00201791C 0349080,Monocyte_PER2,2255); (75,C00201791|C0349080,Colon_RAMP2,2035); (75,C0020179|C0349080,Unknown_CD86,1905); (75,C0020179|C0349080,Monocyte_RILP,1893); (75,C00201 79|C0349080,Monocyte_SNX9,1875); (75,C0020179|C0349080,Endometrial tissue_TVP23CP2,1868); (75,C00201791|C0349080,Monocyte_EIF3G,1705); (75,C00201791|C034 9080,Endometrial tissue_SMC2,1699); (75,C00201791|C0349080,Temporal_cortex_TRIM37,1687); (75,C0020179|C0349080,Colorectum_VPS37B,1656); (75,C00201791|C0349080,Brain white matter_STON1-GTF2A1L,1647); (75,C0020179|C0349080,Endometrial tissue_TMEM159,1635); (75,C00201791|C0349080,Endometrial tissue_GLB1,1550); (75,C0020179|C0349080,Monocyte_PDHX,1494); (75,C0020179|C0349080,Bladder_THEM6,1490); (75,C0020179|C0349080,Temporal_cortex_MYADML2,1475); (75,C0 0201791|C0349080,Brain white matter_CLEC12A,1473); (75,C0020179|C0349080,Macrophage_MEPE,1424); (75,C0020179|C0 349080,Endometrial tissue_PODN,1416); (77,C0020517|C0034525|C0156153|C0156154|C0267166|C0348099|C0348 101|C0348674|C0348676|C0348738|C0413022|C0494761|C0494762|C0494807|C0496106|C054 6822,Whole_blood_AKIRIN2,2911); (77,C0020517|C0034525|C0156153|C0156154|C0267166| C0348099|C0348101|C0348674|C0348676|C0348738|C0413022|C0494761|C0494762|C049480 7|C0496106|C0546822,Monocyte_PER2,2255); (77,C0020517|C0034525|C0156153|C01561541C 0267166|C0348099|C0348101|C0348674|C0348676|C0348738|C0413022|C0494761|C0494762| C0494807|C0496106|C0546822,Colon_RAMP2,2035); (77,C0020517|C0034525|C0156153|C01 494762|C0494807|C0496106|C0546822,Unknown_CD86,1905); (77,C0020517|C0034525|C015 6153|C0156154|C0267166|C0348099|C0348101|C0348674|C0348676|C0348738|C0413022|C04 94761|C0494762|C0494807|C0496106|C0546822,Monocyte_RILP,1893); (77,C0020517|C00345 25|C0156153|C0156154|C0267166|C0348099|C0348101|C0348674|C0348676|C0348738|C0413 022|C049476|C0494762|C0494807|C0496106|C0546822,Monocyte_SNX9,1875); (77,C002051 7|C0034525|C0156153|C0156154|C0267166|C0348099|C0348101|C0348674|C0348676|C03487 38|C0413022|C0494761|C0494762|C0494807|C0496106|C0546822,Endometrial tissue_TVP23CP2,1868); (77,C0020517|C0034525|C0156153|C0156154|C0267166|C0348099|C 0348101|C0348674|C0348676|C0348738|C0413022|C0494761|C0494762|C0494807|C0496106| C0546822,Monocyte_EIF3G,1705); (77,C0020517|C0034525|C0156153|C0156154|C0267166|C 0348099|C0348101|C0348674|C0348676|C0348738|C0413022|C0494761|C0494762|C0494807| C0496106|C0546822,Endometrial tissue_SMC2,1699); (77,C0020517|C0034525|C0156153|C0156154|C0267166|C0348099|C0348 101|C0348674|C0348676|C0348738|C0413022|C0494761|C0494762|C0494807|C0496106|C054 6822,Temporal_cortex_TRIM37,1687); (77,C0020517|C0034525|C0156153|C01561541|C026716 6|C0348099|C0348101|C0348674|C0348676|C0348738|C0413022|C0494761|C0494762|C04948 07|C0496106|C0546822,Colorectum_VPS37B,1656); (77,C0020517|C0034525|C0156153|C0156 154|C0267166|C0348099|C0348101|C0348674|C0348676|C0348738|C0413022|C0494761|C049 4762|C04948071|C0496106|C0546822,Brain white matter_STON1-GTF2A1L,1647); (77,C0020517|C0034525|C0156153|C0156154|C0267166|C0348099|C034810 1|C0348674|C0348676|C0348738|C0413022|C0494761|C0494762|C0494807|C0496106|C05468 22,Endometrial tissue_TMEM159,1635); (77,C0020517|C0034525|C0156153|C0156154|C0267166|C0348099|C 0348101|C0348674|C0348676|C0348738|C0413022|C0494761|C0494762|C0494807|C0496106| C0546822,Endometrial tissue_GLB1,1550); (77,C0020517|C0034525|C0156153|C0156154|C0267166|C0348099|C0348 101|C0348674|C0348676|C0348738|C0413022|C0494761|C0494762|C0494807|C0496106|C054 6822,Monocyte_PDHX,1494); (77,C0020517|C0034525|C0156153|C0156154|C0267166|C03480 99|C0348101|C0348674|C0348676|C0348738|C0413022|C0494761|C0494762|C0494807|C0496 106|C0546822,Bladder_THEM6,1490); (77,C0020517|C0034525|C0156153|C0156154|C026716 6|C0348099|C0348101|C0348674|C0348676|C0348738|C0413022|C0494761|C0494762|C04948 071|C0496106|C0546822,Temporal_cortex_MYADML2,1475); (77,C0020517|C0034525|C01561 53|C0156154|C0267166|C0348099|C0348101|C0348674|C0348676|C0348738|C0413022|C0494 761|C0494762|C0494807|C0496106|C0546822,Brain white matter_CLEC12A,1473); (77,C00205171|C0034525|C0156153|C0156154|C0267166|C03480991C 0348101|C0348674|C0348676|C0348738|C0413022|C0494761|C0494762|C0494807|C0496106| C0546822,Macrophage_MEPE,1424); (77,C0020517|C0034525|C0156153|C0156154|C0267166| C0348099|C0348101|C0348674|C0348676|C0348738|C0413022|C0494761|C0494762|C049480 7|C0496106|C0546822,Endometrial tissue_PODN,1416); (83,C00213641|C0405581|C0476525,Whole_blood_AKIRIN2,2911); (83,C0 021364|C0405581|C0476525,Monocyte_PER2,2255); (83,C0021364|C0405581|C0476525,Colon_RAMP2,2035); (83,C0021364|C040558|C0476525,Unknown_CD86,1905); (83,C0021364|C04 0558|C0476525,Monocyte_RILP,1893); (83,C0021364|C040558|C0476525,Monocyte_SNX9, 1875); (83,C00213641|C0405581|C0476525,Endometrial tissue_TVP23CP2,1868); (83,C00213641|C0405581|C0476525,Monocyte_EIF3G,1705); (83,C002 13641|C0405581|C0476525,Endometrial tissue_SMC2,1699); (83,C00213641|C0405581|C0476525,Temporal_cortex_TRIM37,1687); (83, C00213641|C0405581|C0476525,Colorectum_VPS37B,1656); (83,C00213641|C0405581|C047652 5,Brain white matter_STON1-GTF2A1L,1647); (83,C00213641|C0405581|C0476525,Endometrial tissue_TMEM159,1635); (83,C00213641|C040558|C0476525,Endometrial tissue_GLB1,1550); (83,C00213641|C0405581|C0476525,Monocyte PDHX,1494); (83,C0021364|C0405581|C0476525,Bladder_THEM6,1490); (83,C0021364|C0405581|C0476525,Temporal_co rtex_MYADML2,1475); (83,C00213641|C0405581|C0476525,Brain white matter_CLEC12A,1473); (83,C0021364|C040558|C0476525,Macrophage_MEPE,1424); (83,C0 0213641|C0405581|C0476525,Endometrial tissue_PODN,1416); (86,C0022650,Whole_blood_AKIRIN2,2911); (86,C0022650,Whole_blood_AKIRIN2,2911); (86,C0022650,Monocyte_PER2,2255); (86,C0022650,Monocyte_PER2,2255); (86,C0022650,Colon_RAMP2,2035); (86,C0022650,Colon_RAMP2,2035); (86,C0022650,Unkn own_CD86,1905); (86,C0022650,Unknown_CD86,1905); (86,C0022650,Monocyte_RILP,1893); (86,C0022650,Monocyte_RILP,1893); (86,C0022650,Monocyte_SNX9,1875); (86,C0022650,Mo nocyte_SNX9,1875); (86,C0022650,Endometrial tissue_TVP23CP2,1868); (86,C0022650,Endometrial tissue_TVP23CP2,1868); (86,C0022650,Monocyte_EIF3G,1705); (86,C0022650,Monocyte_EIF3 G,1705); (86,C0022650,Endometrial tissue_SMC2,1699); (86,C0022650,Endometrial tissue_SMC2,1699); (86,C0022650,Temporal_cortex_TRIM37,1687); (86,C0022650,Temporal_c ortex_TRIM37,1687); (87,C0022661|C0041349|C0268731|C0348642|C0477312|C0495063|C084 8548,Whole_blood_AKIRIN2,2911); (87,C0022661|C0041349|C0268731|C0348642|C0477312| C0495063|C0848548,Monocyte_PER2,2255); (87,C0022661|C0041349|C0268731|C0348642|C0 477312|C0495063|C0848548,Colon_RAMP2,2035); (87,C0022661|C0041349|C0268731|C03486 42|C0477312|C0495063|C0848548,Unknown_CD86,1905); (87,C002266|C0041349|C0268731| C0348642|C0477312|C0495063|C0848548,Monocyte_RILP,1893); (87,C0022661|C0041349|C0 268731|C0348642|C0477312|C0495063|C0848548,Monocyte_SNX9,1875); (87,C0022661|C004 13491|C0268731|C0348642|C0477312|C0495063|C0848548,Endometrial tissue_TVP23CP2,1868); (87,C0022661|C0041349|C0268731|C0348642|C0477312|C0495063|C 0848548,Monocyte_EIF3G,1705); (87,C0022661|C0041349|C0268731|C0348642|C0477312|C04 95063|C0848548,Endometrial tissue_SMC2,1699); (87,C0022661|C0041349|C0268731|C0348642|C0477312|C0495063|C0848 548,Temporal_cortex_TRIM37,1687); (87,C002266|C00413491|C026873|C0348642|C0477312|C0495063|C0848548,Colorectum_VPS37B,1656); (87,C0022661|C00413491|C0268731|C034864 2|C0477312|C0495063|C0848548,Brain white matter_STON1-GTF2A1L,1647); (87,C002266|C0041349|C026873|C0348642|C0477312|C0495063|C084854 8,Endometrial tissue_TMEM159,1635); (87,C0022661|C0041349|C0268731|C0348642|C0477312|C0495063|C 0848548,Endometrial tissue_GLB1,1550); (87,C0022661|C0041349|C0268731|C0348642|C0477312|C0495063|C0848 548,Monocyte_PDHX,1494); (87,C0022661|C0041349|C0268731|C0348642|C0477312|C049506 3|C0848548,Bladder_THEM6,1490); (87,C0022661|C0041349|C0268731|C0348642|C0477312| C0495063|C0848548,Temporal_cortex_MYADML2,1475); (87,C0022661|C0041349|C0268731| C0348642|C0477312|C0495063|C0848548,Brain white matter_CLEC12A,1473); (87,C0022661|C0041349|C0268731|C0348642|C0477312|C0495063|C 0848548,Macrophage_MEPE,1424); (87,C0022661|C0041349|C0268731|C0348642|C0477312|C 0495063|C0848548,Endometrial tissue_PODN,1416); (92,C00255171|C02636601|C0348791,Whole_blood_AKIRIN2,2911); (92,C0 0255171|C02636601|C0348791,Monocyte PER2,2255); (92,C0025517|C02636601|C0348791,Colon_RAMP2,2035); (92,C0025517|C0263660|C0348791,Unknown_CD86,1905); (92,C0025517|C02 63660|C0348791,Monocyte_RILP,1893); (92,C0025517|C0263660|C0348791,Monocyte_SNX9, 1875); (92,C00255171|C02636601|C0348791,Endometrial tissue_TVP23CP2,1868); (92,C0025517|C02636601|C0348791,Monocyte_EIF3G,1705); (92,C002 55171|C02636601|C0348791,Endometrial tissue_SMC2,1699); (92,C0025517|C02636601|C0348791,Temporal_cortex_TRIM37,1687); (92, C00255171|C02636601|C0348791,Colorectum_VPS37B,1656); (92,C0025517|C02636601|C034879 1,Brain white matter_STON1-GTF2A1L,1647); (92,C0025517|C02636601|C0348791,Endometrial tissue_TMEM159,1635); (92,C0025517|C02636601|C0348791,Endometrial tissue_GLB1,1550); (92,C0025517|C02636601|C0348791,Monocyte PDHX,1494); (92,C0025517| C0263660|C0348791,Bladder_THEM6,1490); (92,C0025517|C0263660|C0348791,Temporal_co rtex_MYADML2,1475); (92,C0025517|C02636601|C0348791,Brain white matter_CLEC12A,1473); (92,C0025517|C0263660|C0348791,Macrophage_MEPE,1424); (92,C0 0255171|C02636601|C0348791,Endometrial tissue_PODN,1416); (95,C0026896|C01541991|C0494501|C0495465,Whole_blood_AKIRIN2,29 11); (95,C0026896|C01541991|C0494501|C0495465,Monocyte_PER2,2255); (95,C0026896|C015 41991|C0494501|C0495465,Colon_RAMP2,2035); (95,C0026896|C01541991|C0494501|C0495465,Unknown_CD86,1905); (95,C0026896|C0154199|C049450|C0495465,Monocyte_RILP,1893); (95,C0026896|C0154199|C049450|C0495465,Monocyte_SNX9,1875); (95,C0026896|C0154199|C049450|C0495465,Endometrial tissue_TVP23CP2,1868); (95,C0026896|C0154199|C0494501|C0495465,Monocyte_EIF3G,1705); (95,C0026896|C0154199|C049450|C0495465,Endometrial tissue_SMC2,1699); (95,C0026896|C0154199|C0494501|C0495465,Temporal_cortex_TRIM37, 1687); (95,C0026896|C0154199|C0494501|C0495465,Colorectum_VPS37B,1656); (95,C002689 6|C01541991|C0494501|C0495465,Brain white matter_STON1-GTF2A1L,1647); (95,C0026896|C0154199|C049450|C0495465,Endometrial tissue_TMEM159,1635); (95,C0026896|C01541991|C049450|C0495465,Endometrial tissue_GLB1,1550); (95,C0026896|C0154199|C0494501|C0495465,Monocyte PDHX,1494); (95, C0026896|C0154199|C049450|C0495465,Bladder_THEM6,1490); (95,C0026896|C0154199|C0 494501|C0495465,Temporal_cortex_MYADML2,1475); (95,C0026896|C01541991|C0494501|C0 495465,Brain white matter_CLEC12A,1473); (95,C0026896|C0154199|C049450|C0495465,Macrophage_MEPE,14 24); (95,C0026896|C01541991|C0494501|C0495465,Endometrial tissue_PODN,1416); (97,C0027531|C0037929|C0411063|C0452049|C0452050|C045205|C0478 224|C0478257|C0694460|C1331979,Whole_blood_AKIRIN2,2911); (97,C00027531|C0037929|C 0411063|C0452049|C0452050|C0452051|C0478224|C0478257|C0694460|C1331979,Monocyte_PER2,2255); (97,C002753|C0037929|C0411063|C0452049|C0452050|C045205|C0478224|C0 478257|C0694460|C1331979,Colon_RAMP2,2035); (97,C0027531|C0037929|C0411063|C04520 49|C0452050|C0452051|C0478224|C0478257|C0694460|C1331979,Unknown_CD86,1905); (97, C00027531|C0037929|C0411063|C0452049|C0452050|C0452051|C0478224|C0478257|C069446 O|C1331979,Monocyte_RILP,1893); (97,C0027531|C0037929|C0411063|C0452049|C0452050|C 37929|C0411063|C0452049|C0452050|C0452051|C0478224|C478257|C06944601331979,En dometrial tissue_TVP23CP2,1868); (97,C0027531|C0037929|C0411063|C0452049|C0452050|C045205|C 0478224|C0478257|C0694460|C1331979,Monocyte_EIF3G,1705); (97,C0027531|C0037929|C04 11063|C0452049|C0452050|C0452051|C0478224|C0478257|C0694460|C1331979,Endometrial tissue_SMC2,1699); (97,C0027531|C0037929|C0411063|C0452049|C0452050|C0452051|C0478 224|C0478257|C0694460|C1331979,Temporal_cortex_TRIM37,1687); (97,C002753|C00379291|C0411063|C0452049|C0452050|C0452051|C0478224|C0478257|C0694460|C1331979,Colorectu m_VPS37B,1656); (97,C0027531|C0037929|C0411063|C0452049|C0452050|C0452051|C04782 241|C04782571|C0694460|C1331979,Brain white matter_STON1-GTF2A1L,1647); (97,C002753|C0037929|C0411063|C0452049|C0452050|C045205|C047822 4|C0478257|C0694460|C1331979,Endometrial tissue_TMEM159,1635); (97,C0027531|C0037929|C0411063|C0452049|C0452050|C0452051|C 04782241|C04782571|C0694460|C1331979,Endometrial tissue_GLB1,1550); (97,C0027531|C0037929|C0411063|C0452049|C0452050|C0452051|C0478 224|C0478257|C0694460|C1331979,Monocyte_PDHX,1494); (97,C0027531|C0037929|C041106 3|C0452049|C0452050|C0452051|C0478224|C0478257|C0694460|C1331979,Bladder_THEM6, 1490); (97,0027531|C0037929|C010 3|C002|C0478257|C0694460|C1331979,Temporal_cortex_MYADML2,1475); (97,C0027531|C00379291|C0411063| C0452049|C0452050|C0452051|C0478224|C0478257|C0694460|C1331979,Brain white matter_CLEC12A,1473); (97,C0027531|C0037929|C0411063|C0452049|C0452050|C0452051|C 0478224|C0478257|C0694460|C1331979,Macrophage_MEPE,1424); (97,C0027531|C0037929|C 0411063|C0452049|C0452050|C0452051|C0478224|C0478257|C0694460|C1331979,Endometri al tissue_PODN,1416); (100,C0029456|C0029458|C00296941|C00317071|C0042870|C0152256|C015 8447|C0264115|C0451868|C0451869|C0451870|C0451871|C0451872|C04518731|C0451881|C04 51882|C0451883|C0451884|C0477673|C0477674|C0494808|C0494958|C0494964|C0494997|C0 494998|C0495002|C0495020|C0495120|C0495123|C0521170|C0694460|C0694523,Whole_bloo d_AKIRIN2,2911); (100,C0029456|C0029458|C0029694|C0031707|C0042870|C0152256|C0158 447|C0264115|C0451868|C0451869|C0451870|C0451871|C0451872|C0451873|C0451881|C045 1882|C0451883|C0451884|C0477673|C0477674|C0494808|C0494958|C0494964|C0494997|C04 94998|C0495002|C0495020|C0495120|C0495123|C0521170|C0694460|C0694523,Monocyte_P ER2,2255); (100,C0029456|C0029458|C0029694|C0031707|C0042870|C0152256|C0158447|C02 64115|C0451868|C04518691|C0451870|C0451871|C0451872|C0451873|C0451881|C0451882|C0 451883|C0451884|C0477673|C0477674|C0494808|C0494958|C0494964|C0494997|C0494998|C 0495002|C0495020|C0495120|C0495123|C0521170|C0694460|C0694523,Colon_RAMP2,2035) ; (100,C0029456|C0029458|C0029694|C0031707|C0042870|C0152256|C0158447|C0264115|C04 51868|C0451869|C0451870|C0451871|C0451872|C0451873|C0451881|C0451882|C0451883|C0 451884|C0477673|C0477674|C0494808|C0494958|C0494964|C0494997|C0494998|C0495002|C 0495020|C0495120|C0495123|C0521170|C0694460|C0694523,Unknown_CD86,1905); (100,C0 029456|C0029458|C0029694|C0031707|C0042870|C0152256|C0158447|C0264115|C0451868|C 0451869|C0451870|C0451871|C0451872|C0451873|C0451881|C0451882|C0451883|C0451884| C0477673|C0477674|C0494808|C0494958|C0494964|C0494997|C0494998|C0495002|C049502 O|C04951201|C0495123|C05211701|C06944601|C0694523,Monocyte_RILP,1893); (100,C00294561|C0029458|C0029694|C0031707|C0042870|C0152256|C0158447|C0264115|C0451868|C045186 9|C0451870|C0451871|C0451872|C0451873|C0451881|C0451882|C0451883|C0451884|C04776 73|C0477674|C0494808|C0494958|C0494964|C0494997|C0494998|C0495002|C0495020|C0495 1201|C0495123|1|C05211701|C06944601|C0694523,Monocyte_SNX9,1875); (100,C0029456| C00294 58|C0029694|C0031707|C0042870|C0152256|C0158447|C0264115|C0451868|C0451869|C0451 870|C0451871|C0451872|C0451873|C0451881|C0451882|C0451883|C0451884|C0477673|C047 76741|C0494808| C0494958|C04949641|C0494997|C0494998|C0495002| C04950201|C04951201|C04 95123|C05211701|C06944601|C0694523,Endometrial tissue_TVP23CP2,1868); (100,C0029456|C0029458|C0029694|C0031707|C0042870|C0152256| C0158447|C0264115|C0451868|C0451869|C0451870|C0451871|C0451872|C0451873|C045188 1|C0451882|C0451883|C0451884|C0477673|C0477674|C0494808|C0494958|C0494964|C04949 97|C0494998|C0495002|C0495020|C0495120|C0495123|C0521170|C0694460|C0694523,Mono cyte_EIF3G,1705); (100,C0029456|C0029458|C0029694|C0031707|C0042870|C0152256|C0158 447|C0264115|C0451868|C0451869|C0451870|C0451871|C0451872|C0451873|C0451881|C045 1882|C0451883|C0451884|C0477673|C0477674|C0494808|C0494958|C0494964|C0494997|C04 94998|C0495002|C0495020|C0495120|C0495123|C0521170|C0694460|C0694523,Endometrial tissue_SMC2,1699); (100,C0029456|C0029458|C0029694|C0031707|C0042870|C0152256|C015 8447|C0264115|C0451868|C0451869|C0451870|C0451871|C0451872|C0451873|C0451881|C04 51882|C0451883|C0451884|C0477673|C0477674|C0494808|C0494958|C0494964|C0494997|C0 494998|C0495002|C0495020|C0495120|C0495123|C0521170|C0694460|C0694523,Temporal_c ortex_TRIM37,1687); (100,C0029456|C0029458|C0029694|C0031707|C0042870|C0152256|C01 58447|C0264115|C0451868|C0451869|C0451870|C0451871|C0451872|C0451873|C0451881|C0 451882|C0451883|C0451884|C0477673|C0477674|C0494808|C0494958|C0494964|C0494997|C 04949981|C0495002|C04950201|C04951201|C0495123|C0521170|C0694460|C0694523,Colorectu m_VPS37B,1656); (100,C0029456|C0029458|C0029694|C0031707|C0042870|C0152256|C0158 447|C0264115|C0451868|C0451869|C0451870|C0451871|C0451872|C0451873|C0451881|C045 1882|C0451883|C0451884|C0477673|C0477674|C0494808|C0494958|C0494964|C0494997|C04 94998|C0495002|C0495020|C0495120|C0495123|C0521170|C0694460|C0694523,Brain white matter_STON1-GTF2A1L,1647); (100,C0029456|C0029458|C0029694|C0031707|C0042870|C0152256|C01584 47|C0264115|C0451868|C0451869|C0451870|C0451871|C0451872|C0451873|C0451881|C0451 882|C0451883|C0451884|C0477673|C0477674|C0494808|C0494958|C0494964|C0494997|C049 4998|C0495002|C0495020|C0495120|C0495123|C0521170|C0694460|C0694523,Endometrial tissue_TMEM159,1635); (100,C0029456|C0029458|C0029694|C0031707|C0042870|C0152256| C0158447|C0264115|C0451868|C0451869|C0451870|C0451871|C0451872|C0451873|C045188 1|C0451882|C0451883|C0451884|C0477673|C0477674|C0494808|C0494958|C0494964|C04949 97|C0494998|C0495002|C0495020|C0495120|C0495123|C0521170|C0694460|C0694523,Endo metrial tissue_GLB1,1550); (100,C0029456|C0029458|C0029694|C0031707|C0042870|C0152256|C015 8447|C0264115|C0451868|C0451869|C0451870|C0451871|C0451872|C0451873|C0451881|C04 51882|C0451883|C0451884|C0477673|C0477674|C0494808|C0494958|C0494964|C0494997|C0 494998|C0495002|C0495020|C0495120|C0495123|C0521170|C0694460|C0694523,Monocyte_P DHX,1494); (100,C0029456|C0029458|C0029694|C0031707|C0042870|C0152256|C0158447|C0 264115|C0451868|C0451869|C0451870|C0451871|C0451872|C0451873|C0451881|C0451882|C 0451883884|C0477673|C0477674|C0494808|C0494958|C0494964|C04949971|C0494998| C0495002|C0495020|C0495120|C0495123|C0521170|C0694460|C0694523,Bladder_THEM6,14 90); (100,C00294561|C0029458|C0029694|C0031707|C0042870|C0152256|C0158447|C0264115| C0451868|C0451869|C0451870|C0451871|C0451872|C0451873|C0451881|C0451882|C045188 3|C0451884|C0477673|C0477674|C0494808|C0494958|C0494964|C0494997|C0494998|C04950 02|C0495020|C0495120|C0495123|C0521170|C0694460|C0694523,Temporal_cortex_MYADM L2,1475); (100,C0029456|C0029458|C0029694|C0031707|C0042870|C0152256|C0158447|C026 4115|C0451868|C0451869|C0451870|C0451871|C0451872|C0451873|C0451881|C0451882|C04 51883|C0451884|C0477673|C0477674|C0494808|C0494958|C0494964|C0494997|C0494998|C0 495002|C0495020|C0495120|C0495123|C0521170|C0694460|C0694523,Brain white matter_CLEC12A,1473); (100,C0029456|C00294581|C00296941|C0031707|C0042870|C01522561|C0158447|C0264115|C0451868|C0451869|C0451870|C0451871|C0451872|C0451873|C045188 1|C0451882|C0451883|C0451884|C0477673|C0477674|C0494808|C0494958|C0494964|C04949 971|C04949981|C0495002|C04950201|C04951201|C0495123|C05211701|C06944601|C0694523,Macro phage_MEPE,1424); (100,C0029456|C0029458|C0029694|C0031707|C00428701|C0152256|C015 8447|C0264115|C0451868|C0451869|C0451870|C0451871|C0451872|C0451873|C0451881|C04 51882|C0451883|C0451884|C0477673|C0477674|C0494808|C0494958|C0494964|C0494997|C0 494998|C0495002|C0495020|C0495120|C0495123|C0521170|C0694460|C0694523,Endometrial tissue_PODN,1416); (101,C0029882|C0029888|C0152874|C0155415|C0155421|C0155440|C015 5441|C0155460|C0161744|C0271431|C0271446|C0395849|C0477443|C0477444|C0477445|C04 77446|C0477447|C0477448|C0494087|C0694493|C1455742,Whole_blood_AKIRIN2,2911); (10 1,C0029882|C0029888|C0152874|C0155415|C0155421|C0155440|C0155441|C0155460|C01617 44|C0271431|C0271446|C0395849|C0477443|C04774441|C0477445|C0477446|C0477447|C0477 448|C0494087|C0694493|C1455742,Monocyte_PER2,2255); (1001,C0029882|C0029888|C01528 74|C0155415|C0155421|C0155440|C0155441|C0155460|C0161744|C0271431|C0271446|C0395 8491|C0477443|1|C04774441|C0477445|1|C0477446| C0477447|1|C0477448|1|C0494087| C0694493|C145 5742,Colon_RAMP2,2035); (1001,C0029882| C0029888| C01528741|C0155415|1|C0155421|1|C015544 0|C0155441|C0155460|C0161744|C0271431|C0271446|C0395849|C0477443|C0477444|C04774 45|C0477446|C0477447|C0477448|C0494087|C0694493|C1455742,Unknown_CD86,1905); (10 1,C0029882|C0029888|C0152874|C0155415|C0155421|C0155440|C0155441|C0155460|C01617 44|C0271431|C0271446|C0395849|C0477443|C0477444|C0477445|C0477446|C0477447|C0477 448|C0494087|C0694493|C1455742,Monocyte_RILP,1893); (101,C0029882|C0029888|C015287 41|C0155415|1|C0155421|1|C01554401|C0155441|1|C01554601|C01617441|C0271431|1|C0271446| C03958 49|C0477443|C0477444|C0477445|C0477446|C0477447|C0477448|C0494087|C0694493|C1455 742,Monocyte_SNX9,1875); (101,C0029882|C0029888|C0152874|C0155415|C0155421|C01554 40|C0155441|C0155460|C0161744|C0271431|C0271446|C0395849|C0477443|C0477444|C0477 445|C0477446|C04774471|C04774481|C04940871|C0694493|C1455742,Endometrial tissue_TVP23CP2,1868); (101,C0029882|C0029888|C0152874|C0155415|C0155421|C0155440| C0155441|C0155460|C0161744|C0271431|C0271446|C0395849|C0477443|C0477444|C047744 5|1|C0477446| C04774471|C0477448|1|C0494087|1|C0694493|C1455742,Monocyte_EIF3 G,1705); (101,C0029882|C0029888|C0152874|C0155415|C0155421|C0155440|C0155441|C0155460|C016174 4|C0271431|C0271446|C0395849|C0477443|C0477444|C0477445|C0477446|C0477447|C04774 481|C04940871|C0694493|C1455742,Endometrial tissue_SMC2,1699); (101,C0029882|C0029888|C0152874|C0155415|C0155421|C0155440|C015 5441|C0155460|C0161744|C0271431|C0271446|C0395849|C0477443|C0477444|C0477445|C04 77446|C0477447|C0477448|C0494087|C0694493|C1455742,Temporal_cortex_TRIM37,1687); (101,C0029882|C0029888|C0152874|C0155415|C0155421|C0155440|C0155441|C0155460|C016 1744|C0271431|C0271446|C0395849|C0477443|C04774441|C0477445|C0477446|C0477447|C04 774481|C04940871|C0694493|C1455742,Colorectum_VPS37B,1656); (101,C0029882|C0029888|C 0152874|C0155415|C0155421|C0155440|C0155441|C0155460|C0161744|C0271431|C0271446| C0395849|C0477443|C0477444|C0477445|C0477446|C0477447|C0477448|C0494087|C069449 3|C1455742,Brain white matter_STON1-GTF2A1L,1647); (1001,C0029882| C0029888|C01528741|C0155415|C0155421|1|C01554401|C01554 41|C0155460|C0161744|C0271431|C0271446|C0395849|C0477443|C0477444|C0477445|C0477 446|C0477447|C0477448|C0494087|C0694493|C1455742,Endometrial tissue_TMEM159,1635); (101,C0029882|C0029888|C0152874|C0155415|C0155421|C0155440| C0155441|C0155460|C0161744|C0271431|C0271446|C0395849|C0477443|C04774443|C047744 5|C0477446|C0477447|C0477448|C0494087|C0694493|C1455742,Endometrial tissue_GLB1,1550); (101,C0029882|C0029888|C0152874|C0155415|C0155421|C0155440|C015 5441|C0155460|C0161744|C0271431|C0271446|C0395849|C0477443|C0477444|C0477445|C04 77446|C0477447|C0477448|C0494087|C0694493|C1455742,Monocyte PDHX,1494); (101,C002 9882|C0029888|C0152874|C0155415|C0155421|C0155440|C0155441|C0155460|C0161744|C02 714311|C02714461|C0395849|C0477443|C0477444|C0477445|C0477446|C0477447|C0477448|C0 494087|C0694493|C1455742,Bladder_THEM6,1490); (101,C0029882|C0029888|C0152874|C01 55415|C0155421|C0155440|C0155441|C0155460|C0161744|C0271431|C0271446|C0395849|C0 477443|C0477444|C0477445|C0477446|C0477447|C0477448|C0494087|C0694493|C1455742,T emporal_cortex_MYADML2,1475); (101,C0029882|C0029888|C0152874|C0155415|C0155421| C0155440|C0155441|C0155460|C0161744|C0271431|C0271446|C0395849|C0477443|C047744 4|C0477445|C0477446|C0477447|C0477448|C0494087|C0694493|C1455742,Brain white matter_CLEC12A,1473); (101,C0029882|C0029888|C0152874|C0155415|C0155421|C01554401|C0155441|C0155460|C0161744|C0271431|C0271446|C0395849|C0477443|C0477444|C047744 5|1|C0477446| C04774471|C0477448|1|C0494087|1|C0694493|C1455742,Macrophage_MEPE,1424); (1 01,C0029882|C0029888|C0152874|C0155415|C0155421|C0155440|C0155441|C0155460|C0161 744|C027143446|C0395849|C0477443|C0477444|C0477445|C0477446|C0477447|C047 7448|C0494087|C0694493|C1455742,Endometrial tissue_PODN,1416); (103,C0030567|C1828079,Whole_blood_AKIRIN2,2911); (103,C00305671|C1828079,Monocyte PER2,2255); (103,C0030567|C1828079,Colon_RAMP2,2035); (103,C0030 5671|C1828079,Unknown_CD86,1905); (103,C0030567|C1828079,Monocyte_RILP,1893); (103, C0030567|C1828079,Monocyte_SNX9,1875); (103,C0030567|C1828079,Endometrial tissue_TVP23CP2,1868); (103,C0030567|C1828079,Monocyte_EIF3G,1705); (103,C00305671|C1 828079,Endometrial tissue_SMC2,1699); (103,C0030567|C1828079,Temporal_cortex_TRIM37,1687); (103,C003056 71|C1828079,Colorectum_VPS37B,1656); (103,C0030567|C1828079,Brain white matter_STON1-GTF2A1L,1647); (103,C0030567|C1828079,Endometrial tissue_TMEM159,1635); (103,C0030567|C1828079,Endometrial tissue_GLB1,1550); (103,C0030567|C1828079,Monocyte PDHX,1494); (103,C0030567|C18280 79,Bladder_THEM6,1490); (103,C0030567|C1828079,Temporal_cortex_MYADML2,1475); (10 3,C0030567|C1828079,Brain white matter_CLEC12A,1473); (103,C0030567|C1828079,Macrophage_MEPE,1424); (103,C0030567| C1828079,Endometrial tissue_PODN,1416); (104,C0030809,Whole_blood_AKIRIN2,2911); (104,C0030809,Monocyte_PER2,2255); (104,C0030809,Colon_RAMP2,2035); (104,C0030809,Unknown_CD86,1905); (104,C0030809,Monocyte_RILP,1893); (104,C0030809,Monocyte_SNX9,1875); (104,C0030809,End ometrial tissue_TVP23CP2,1868); (104,C0030809,Monocyte_EIF3G,1705); (104,C0030809,Endometrial tissue_SMC2,1699); (104,C0030809,Temporal_cortex_TRIM37,1687); (104,C0030809,Colorectu m_VPS37B,1656); (104,C0030809,Brain white matter_STON1-GTF2A1L,1647); (104,C0030809,Endometrial tissue_TMEM159,1635); (104,C0030809,Endometrial tissue_GLB1,1550); (104,C0030809,Monocyte_PDHX,1494); (104,C0030809,Bladder_THEM6, 1490); (104,C0030809,Temporal_cortex_MYADML2,1475); (104,C0030809,Brain white matter_CLEC12A,1473); (104,C0030809,Macrophage_MEPE,1424); (104,C0030809,Endometria 1 tissue_PODN,1416); (105,C0031090|C1314006,Whole_blood_AKIRIN2,2911); (105,C00310901|C1314006,Monocyte PER2,2255); (105,C0031090|C1314006,Colon_RAMP2,2035); (105,C0031 090|C1314006,Unknown_CD86,1905); (105,C0031090|C1314006,Monocyte_RILP,1893); (105, C0031090|C1314006,Monocyte_SNX9,1875); (105,C0031090|C1314006,Endometrial tissue_TVP23CP2,1868); (105,C0031090|C1314006,Monocyte_EIF3G,1705); (105,C0031090|C1 314006,Endometrial tissue_SMC2,1699); (105,C0031090|C1314006,Temporal_cortex_TRIM37,1687); (105,C003109 0|C1314006,Colorectum_VPS37B,1656); (105,C0031090|C1314006,Brain white matter_STON1-GTF2A1L,1647); (105,C0031090|C1314006,Endometrial tissue_TMEM159,1635); (105,C0031090|C1314006,Endometrial tissue_GLB1,1550); (105,C0031090|C1314006,Monocyte_PDHX,1494); (105,C0031090|C13140 06,Bladder_THEM6,1490); (105,C0031090|C1314006,Temporal_cortex_MYADML2,1475); (10 5,C0031090|C1314006,Brain white matter_CLEC12A,1473); (105,C0031090|C1314006,Macrophage_MEPE,1424); (105,C00310901|C1314006,Endometrial tissue_PODN,1416); (106,C0035435,Whole_blood_AKIRIN2,2911); (106,C0035435,Monocyte_PER2,2255); (106,C0035435,Colon_RAMP2,2035); (106,C0035435,Unknown_CD86,1905); (106,C0035435,Monocyte_RILP,1893); (106,C0035435,Monocyte_SNX9,1875); (106,C0035435,End ometrial tissue_TVP23CP2,1868); (106,C0035435,Monocyte_EIF3G,1705); (106,C0035435,Endometrial tissue_SMC2,1699); (106,C0035435,Temporal_cortex_TRIM37,1687); (106,C0035435,Colorectu m_VPS37B,1656); (106,C0035435,Brain white matter_STON1-GTF2A1L,1647); (106,C0035435,Endometrial tissue_TMEM159,1635); (106,C0035435,Endometrial tissue_GLB1,1550); (106,C0035435,Monocyte_PDHX,1494); (106,C0035435,Bladder_THEM6, 1490); (106,C0035435,Temporal_cortex_MYADML2,1475); (106,C0035435,Brain white matter_CLEC12A,1473); (106,C0035435,Macrophage_MEPE,1424); (106,C0035435,Endometria 1 tissue_PODN,1416); (109,C00358541|C04774901|C0477510,Whole_blood_AKIRIN2,2911); (109, C00358541|C04774901|C0477510,Monocyte_PER2,2255); (109,C00358541|C04774901|C0477510,C olon_RAMP2,2035); (109,C0035854|C0477490|C0477510,Unknown_CD86,1905); (109,C00358 54|C0477490|C0477510,Monocyte_RILP,1893); (109,C0035854|C0477490|C0477510,Monocyte_SNX9,1875); (109,C00358541|C04774901|C0477510,Endometrial tissue_TVP23CP2,1868); (109,C0035854|C0477490|C0477510,Monocyte_EIF3G,1705); (109,C0 035854|C04774901|C0477510,Endometrial tissue_SMC2,1699); (109,C0035854|C0477490|C0477510,Temporal_cortex_TRIM37,1687); (10 9,C00358541|C04774901|C0477510,Colorectum_VPS37B,1656); (109,C00358541|C04774901|C0477 510,Brain white matter_STON1-GTF2A1L,1647); (109,C0035854|C0477490|C0477510,Endometrial tissue_TMEM159,1635); (109,C00358541|C04774901|C0477510,Endometrial tissue_GLB1,1550); (109,C00358541|C0477490|C0477510,Monocyte_PDHX,1494); (109,C00358 541|C04774901|C0477510,Bladder_THEM6,1490); (109,C00358541|C04774901|C0477510,Temporal cortex_MYADML2,1475); (109,C00358541|C04774901|C0477510,Brain white matter_CLEC12A,1473); (109,C0035854|C0477490|C0477510,Macrophage_MEPE,1424); (109, C00358541|C04774901|C0477510,Endometrial tissues_PODN,1416); (114,C0036690|C0152486|C0152946|C0152964|C0152965|C0152966|C015 2967|C0269294|C0269936|C0275685|C0276029|C0276063|C0343453|C0348131|C0348133|C03 48152|C03489701|C0349003|C0349004|C0349005|C0349006|C0349008|C0349009|C0 452115|C0452197|C04521984521199|C0477713|C0477736|C091|C90477920C 0477924|C0494048|C0494082|C0495304|C0495408|C0496128|C0496134|C0600327|C0868755| C0870076|C3665339,Whole_blood_AKIRIN2,2911); (114,C0036690|C0152486|C0152946|C015 2964|C0152965|C0152966|C0152967|C0269294|C0269936|C02756851|C0276029|C0276063|C03 43453|C0348131|C0348133|C0348152|C0348970|C0349003|C0349004|C0349005|C0349006|C0 349008|C0349009|C0392110|C0452115|C0452197|C0452198|C0452199|C0477713|C0477736|C 0477851|C0477919|C0477920|C0477924|C0494048|C0494082|C0495304|C0495408|C0496128| C0496134|C0600327|C0868755|C0870076|C3665339,Monocyte_PER2,2255); (114,C0036690|C 0152486|C0152946|C0152964|C0152965|C0152966|C0152967|C0269294|C0269936|C0275685| C0276029|C0276063|C0343453|C0348131|C0348133|C0348152|C0348970|C0349003|C034900 41|C0349005|C0349006|C0349008|C0349009|C0392110|C0452115|C0452197|C0452198|C04521 99|C0477713|C0477736|C0477851|C0477919|C0477920|C0477924|C0494048|C0494082|C0495 304|C0495408|C0496128|C0496134|C0600327|C0868755|C0870076|C3665339,Colon_RAMP2, 2035); (114,C0036690|C0152486|C0152946|C0152964|C0152965|C0152966|C0152967|C026929 4|C0269936|C0275685|C0276029|C0276063|C0343453|C0348131|C0348133|C0348152|C03489 701|C0349003|C0349004|C0349005|C0349006|C0349008|C0349009|C0392110|C0452115|C0452 197|C0452198|C0452199|C0477713|C0477736|C0477851|C0477919|C0477920|C0477924|C049 4048|C0494082|C0495304|C0495408|C0496128|C0496134|C0600327|C0868755|C0870076|C36 65339,Unknown_CD86,1905); (114,C0036690|C0152486|C0152946|C0152964|C0152965|C015 2966|C01529671|C0269294|C0269936|C0275685|C0276029|C0276063|C0343453|C0348131|C03 48133|C0348152|C0348970|C0349003|C0349004|C0349005|C0349006|C0349008|C0349009|C0 392110|C0452115|C0452197|C0452198|C0452199|C0477713|C0477736|C0477851|C0477919|C 0477920|C0477924|C0494048|C0494082|C0495304|C0495408|C0496128|C0496134|C0600327| C0868755|C0870076|C3665339,Monocyte_RILP,1893); (114,C0036690|C0152486|C0152946|C 0152964|C0152965|C0152966|C0152967|C0269294|C0269936|C0275685|C0276029|C0276063| C0343453|C0348131|C0348133|C0348152|C0348970|C0349003|C0349004|C0349005|C034900 6|C0349008|C0349009|C0392110|C0452115|C0452197|C0452198|C0452199|C0477713|C04777 36|C0477851|C0477919|C0477920|C0477924|C0494048|C0494082|C0495304|C0495408|C0496 128|C0496134|C0600327|C08687551|C0870076|C3665339,Monocyte_SNX9,1875); (114,C00366 90|C0152486|C0152946|C0152964|C0152965|C0152966|C0152967|C0269294|C0269936|C0275 685|C0276029|C0276063|C0343453|C0348131|C0348133|C0348152|C0348970|C0349003|C034 9004|C0349005|C0349006|C0349008|C0349009|C0392110|C0452115|C0452197|C0452198|C04 495304|C0495408|C0496128|C0496134|C0600327|C0868755|C0870076|C3665339,Endometrial tissue_TVP23CP2,1868); (114,C0036690|C0152486|C0152946|C0152964|C0152965|C0152966| C0152967|C0269294|C0269936|C0275685|C0276029|C0276063|C0343453|C0348131|C034813 3|C0348152|C0348970|C0349003|C0349004|C0349005|C0349006|C0349008|C0349009|C03921 10|C0452115|C0452197|C0452198|C0452199|C0477713|C0477736|C0477851|C0477919|C0477 920|C0477924|C0494048|C0494082|C0495304|C0495408|C0496128|C0496134|C0600327|C086 8755|C0870076|C3665339,Monocyte_EIF3G,1705); (114,C0036690|C0152486|C0152946|C0152 964|C0152965|C0152966|C0152967|C0269294|C0269936|C0275685|C0276029|C0276063|C034 3453|C0348131|C0348133|C0348152|C0348970|C0349003|C0349004|C0349005|C0349006|C03 49008|C0349009|C0392110|C0452115|C0452197|C0452198|C0452199|C0477713|C0477736|C0 477851|C0477919|C0477920|C0477924|C0494048|C0494082|C0495304|C0495408|C0496128|C 0496134|C0600327|C0868755|C0870076|C3665339,Endometrial tissue_SMC2,1699); (114,C0036690|C0152486|C0152946|C0152964|C0152965|C0152966|C015 2967|C0269294|C0269936|C0275685|C0276029|C0276063|C0343453|C0348131|C0348133|C03 48152|C0348970|C0349003|C0349004|C0349005|C0349006|C0349008|C0349009|C0392110|C0 452115|C0452197|C04521984521199|C0477713|C0477736|C091|C90477920C 0477924|C0494048|C0494082|C0495304|C0495408|C0496128|C0496134|C0600327|C0868755| C0870076|C3665339,Temporal_cortex_TRIM37,1687); (114,C0036690|C0152486|C0152946|C0 152964|C0152965|C0152966|C0152967|C0269294|C0269936|C0275685|C0276029|C0276063|C 0343453|C0348131|C0348133|C0348152|C0348970|C0349003|C0349004|C0349005|C0349006| C0349008|C0349009|C0392110|C0452115|C0452197|C0452198|C0452199|C0477713|C047773 6|C0477851|C0477919|C0477920|C0477924|C0494048|C0494082|C04953041|C0495408|C04961 281|C04961341|C06003271|C0868755|C0870076|C3665339,Colorectum VPS37B,1656); (114,C003 6690|C0152486|C0152946|C0152964|C0152965|C0152966|C0152967|C0269294|C0269936|C02 75685|C0276029|C0276063|C0343453|C0348131|C0348133|C0348152|C0348970|C0349003|C0 349004|C0349005|C0349006|C0349008|C0349009|C0392110|C0452115|C0452197|C0452198|C 0452199|C0477713|C0477736|C0477851|C0477919|C0477920|C0477924|C0494048|C0494082| C0495304|C0495408|C0496128|C0496134|C0600327|C0868755|C0870076|C3665339,Brain white matter_STON1-GTF2A1L,1647); (114,C0036690|C0152486|C0152946|C0152964|C0152965|C0152966|C01529 671|C0269294|C0269936|C0275685|C0276029|C0276063|C0343453|C0348131|C0348133|C0348 152|C0348970|C0349003|C0349004|C0349005|C0349006|C0349008|C0349009|C0392110|C045 2115|C0452197|C0452198|C0452199|C0477713|C0477736|C0477851|C0477919|C0477920|C04 77924|C0494048|C0494082|C0495304|C0495408|C0496128|C0496134|C0600327|C0868755|C0 870076|C3665339,Endometrial tissue_TMEM159,1635); (114,C0036690|C0152486|C0152946|C0152964|C0152965|C0152966| C0152967|C0269294|C0269936|C0275685|C0276029|C0276063|C0343453|C0348131|C034813 3|C0348152|C0348970|C0349003|C0349004|C0349005|C0349006|C0349008|C0349009|C03921 10|C0452115|C0452197|C0452198|C0452199|C0477713|C0477736|C0477851|C0477919|C0477 920|C0477924|C0494048|C0494082|C0495304|C0495408|C0496128|C0496134|C0600327|C086 8755|C0870076|C3665339,Endometrial tissue_GLB1,1550); (114,C0036690|C0152486|C0152946|C0152964|C0152965|C0152966|C015 2967|C0269294|C0269936|C0275685|C0276029|C0276063|C0343453|C0348131|C0348133|C03 48152|C0348970|C0349003|C0349004|C0349005|C0349006|C0349008|C0349009|C0392110|C0 1|C0452197|C0452198|C0452199|C0477713|C0477736|C0477851|C0477919|C0477920|C 0477924|C0494048|C0494082|C0495304|C0495408|C0496128|C0496134|C0600327|C0868755| C0870076|C3665339,Monocyte_PDHX,1494); (114,C00366901|C0152486|C0152946|C0152964| C0152965|C0152966|C0152967|C0269294|C0269936|C0275685|C0276029|C0276063|C034345 3|C0348131|C0348133|C0348152|C0348970|C0349003|C0349004|C0349005|C0349006|C03490 08|C0349009|C0392110|C0452115|C0452197|C0452198|C0452199|C0477713|C0477736|C0477 851|C0477919|C0477920|C0477924|C0494048|C0494082|C0495304|C0495408|C0496128|C049 6134|C0600327|C0868755|C0870076|C3665339,Bladder_THEM6,1490); (114,C0036690|C0152 486|C0152946|C0152964|C0152965|C0152966|C0152967|C0269294|C0269936|C0275685|C027 6029|C0276063|C0343453|C0348131|C0348133|C0348152|C0348970|C0349003|C0349004|C03 49005|C0349006|C0349008|C0349009|C0392110|C0452115|C0452197|C0452198|C0452199|C0 477713|C0477736|C0477851|C0477919|C0477920|C0477924|C0494048|C0494082|C0495304|C 0495408|C0496128|C0496134|C0600327|C0868755|C0870076|C3665339,Temporal_cortex_MY ADML2,1475); (114,C00036690|C0152486|C0152946|C0152964|C0152965|C0152966|C0152967| C0269294|C0269936|C0275685|C0276029|C0276063|C0343453|C0348131|C0348133|C034815 2|C0348970|C0349003|C0349004|C0349005|C0349006|C0349008|C0349009|C0392110|C04521 15|C0452197|C0452198|C0452199|C0477713|C0477736|C0477851|C0477919|C0477920|C0477 924|C0494048|C0494082|C0495304|C0495408|C0496128|C0496134|C0600327|C0868755|C087 0076|C3665339,Brain white matter_CLEC12A,1473); (114,C0036690|C0152486|C0152946|C0152964|C0152965|C0152966|C0152967|C0269294|C0269936|C0275685|C0276029|C0276063|C0343453|C0348131|C034813 3|C0348152|C0348970|C0349003|C0349004|C0349005|C03490081|C0349009|C03921 10|C0452115|C0452197|C0452198|C0452199|C0477713|C0477736|C0477851|C0477919|C0477 8755|C0870076|C3665339,Macrophage_MEPE,1424); (114,C0036690|C0152486|C0152946|C01 52964|C0152965|C0152966|C0152967|C0269294|C0269936|C0275685|C0276029|C0276063|C0 343453|C0348131|C0348133|C0348152|C0348970|C0349003|C0349004|C0349005|C0349006|C 0349008|C0349009|C0392110|C0452115|C0452197|C0452198|C0452199|C0477713|C0477736| C0477851|C0477919|C0477924|C0494048|C0494082|C0495304|C0495408|C049612 8|C04961341|C0600327|C0868755|C0870076|C3665339,Endometrial tissue_PODN,1416); (115,C0038013|C0348540|C0348715|C0409675|C0477606,Whole_blood_AKIRIN2,2911); (115,C0038013|C0348540|C0348715|C0409675|C0477606,Monocyte_PER2,22 55); (115,C0038013|C0348540|C0348715|C0409675|C0477606,Colon_RAMP2,2035); (115,C003 8013|C0348540|C0348715|C0409675|C0477606,Unknown_CD86,1905); (115,C0038013|C0348 540|C0348715|C0409675|C0477606,Monocyte_RILP,1893); (115,C0038013|C0348540|C034871 5|C0409675|C0477606,Monocyte_SNX9,1875); (115,C0038013|C0348540|C0348715|C0409675| C0477606,Endometrial tissue_TVP23CP2,1868); (115,C0038013|C0348540|C0348715|C0409675|C0477606,Monocyte_EIF3G,1705); (115,C0038013|C0348540|C0348715|C0409675|C0477606,Endometrial tissue_SMC2,1699); (115,C0038013|C0348540|C0348715|C0409675|C0477606,Temporal_corte x_TRIM37,1687); (115,C0038013|C0348540|C0348715|C0409675|C0477606,Colorectum_VPS3 7B,1656); (115,C0038013|C03485401|C0348715|C0409675|C0477606,Brain white matter_STON1-GTF2A1L,1647); (115,C0038013|C0348540|C0348715|C0409675|C0477606,Endometrial tissue_TMEM159,1635); (115,C0038013|C0348540|C0348715|C0409675|C0477606,Endometria 1 tissue_GLB1,1550); (115,C0038013|C0348540|C0348715|C0409675|C0477606,Monocyte_PDH X,1494); (115,C0038013|C0348540|C0348715|C0409675|C0477606,Bladder_THEM6,1490); (11 5,C0038013|C0348540|C0348715|C0409675|C0477606,Temporal_cortex_MYADML2,1475); (1 15,C0038013|C03485401|C0348715|C0409675|C0477606,Brain white matter_CLEC12A,1473); (115,C0038013|C0348540|C0348715|C0409675|C0477606,Macrophag e_MEPE,1424); (115,C0038013|C0348540|C0348715|C0409675|C0477606,Endometrial tissue_PODN,1416); (118,C0040156|C0270222|C0477553|C0494276|C0494505,Whole_blood_AKIRIN2,2911); (118,C0040156|C0270222|C0477553|C0494276|C0494505,Monocyte_PER2,22 55); (118,C0040156|C0270222|C0477553|C0494276|C0494505,Colon_RAMP2,2035); (118,C004 0156|C0270222|C0477553|C0494276|C0494505,Unknown_CD86,1905); (118,C0040156|C0270 222|C0477553|C0494276|C0494505,Monocyte_RILP,1893); (118,C0040156|C0270222|C047755 3|C0494276|C0494505,Monocyte_SNX9,1875); (118,C0040156|C0270222|C0477553|C0494276| C0494505,Endometrial tissue_TVP23CP2,1868); (118,C0040156|C0270222|C0477553|C0494276|C0494505,Monocyte_EIF3G,1705); (118,C0040156|C0270222|C0477553|C0494276|C0494505,Endometrial tissue_SMC2,1699); (118,C0040156|C0270222|C0477553|C0494276|C0494505,Temporal_corte x_TRIM37,1687); (118,C0040156|C0270222|C0477553|C0494276|C0494505,Colorectum_VPS3 7B,1656); (118,C0040156|C0270222|C0477553|C0494276|C0494505,Brain white matter_STON1-GTF2A1L,1647); (118,C0040156|C0270222|C0477553|C0494276|C0494505,Endometrial tissue_TMEM159,1635); (118,C0040156|C0270222|C0477553|C0494276|C0494505,Endometria 1 tissue_GLB1,1550); (118,C0040156|C0270222|C0477553|C0494276|C0494505,Monocyte_PDH X,1494); (118,C0040156|C0270222|C0477553|C0494276|C0494505,Bladder_THEM6,1490); (11 8,C0040156|C0270222|C0477553|C0494276|C0494505,Temporal_cortex_MYADML2,1475); (1 18,C0040156|C0270222|C0477553|C0494276|C0494505,Brain white matter_CLEC12A,1473); (118,C0040156|C0270222|C0477553|C0494276|C0494505,Macrophag e_MEPE,1424); (118,C0040156|C0270222|C0477553|C0494276|C0494505,Endometrial tissue_PODN,1416); (119,C0040592|C01531071|C01531081|C0348303,Whole_blood_AKIRIN2,2 911); (119,C0040592|C0153107|C0153108|C0348303,Monocyte_PER2,2255); (119,C0040592|C 0153107|C0153108|C0348303,Colon_RAMP2,2035); (119,C0040592|C0153107|C0153108|C034 8303,Unknown_CD86,1905); (119,C0040592|C0153107|C0153108|C0348303,Monocyte_RILP, 1893); (119,C0040592|C01531071|C01531081|C0348303,Monocyte_SNX9,1875); (119,C00405921|C01531071|C01531081|C0348303,Endometrial tissue_TVP23CP2,1868); (119,C0040592|C0153107|C0153108|C0348303,Monocyte_EIF3G,170 5); (119,C0040592|C0153107|C0153108|C0348303,Endometrial tissue_SMC2,1699); (119,C0040592|C0153107|C0153108|C0348303,Temporal_cortex_TRIM37 ,1687); (119,C0040592|C01531071|C01531081|C0348303,Colorectum_VPS37B,1656); (119,C0040 592|C01531071|C01531081|C0348303,Brain white matter_STON1-GTF2A1L,1647); (119,C0040592|C0153107|C0153108|C0348303,Endometrial tissue_TMEM159,1635); (119,C0040592|C0153107|C0153108|C0348303,Endometrial tissue_GLB1,1550); (119,C0040592|C0153107|C0153108|C0348303,Monocyte_PDHX,1494); (1 19,C0040592|C0153107|C0153108|C0348303,Bladder_THEM6,1490); (119,C0040592|C015310 7|C0153108|C0348303,Temporal_cortex_MYADML2,1475); (119,C0040592|C0153107|C01531 081|C0348303,Brain white matter_CLEC12A,1473); (119,C0040592|C0153107|C0153108|C0348303,Macrophage_MEPE,1 424); (119,C0040592|C01531071|C01531081|C0348303,Endometrial tissue_PODN,1416); (121,C0041333|C0242172|C0348147|C0348149|C0348905|C0451784|C045 1785|C0452162|C0452163|C0494056,Whole_blood_AKIRIN2,2911); (121,C0041333|C0242172| C0348147|C0348149|C0348905|C0451784|C0451785|C0452162|C0452163|C0494056,Monocyt e_PER2,2255); (121,C0041333|C0242172|C0348147|C0348149|C0348905|C0451784|C0451785| C0452162|C0452163|C0494056,Colon_RAMP2,2035); (121,C0041333|C0242172|C0348147|C0 348149|C0348905|C0451784|C0451785|C0452162|C0452163|C0494056,Unknown_CD86,1905) ; (121,C0041333|C0242172|C0348147|C0348149|C0348905|C0451784|C0451785|C0452162|C04 52163|C0494056,Monocyte_RILP,1893); (121,C0041333|C0242172|C0348147|C0348149|C0348 905|C0451784|C0451785|C0452162|C0452163|C0494056,Monocyte_SNX9,1875); (121,C00413 33|C0242172|C0348147|C0348149|C0348905|C0451784|C0451785|C0452162|C0452163|C0494 056,Endometrial tissue_TVP23CP2,1868); (121,C0041333|C0242172|C0348147|C0348149|C0348905|C0451784| C0451785|C0452162|C0452163|C0494056,Monocyte_EIF3G,1705); (121,C0041333|C0242172| C0348147|C0348149|C0348905|C0451784|C0451785|C0452162|C0452163|C0494056,Endometr ial tissue_SMC2,1699); (121,C0041333|C0242172|C0348147|C0348149|C0348905|C0451784|C045 1785|C0452162|C0452163|C0494056,Temporal_cortex_TRIM37,1687); (121,C0041333|C02421 72|C0348147|C0348149|C0348905|C0451784|C0451785|C0452162|C0452163|C0494056,Colore ctum_VPS37B,1656); (121,C0041333|C0242172|C0348147|C0348149|C0348905|C0451784|C04 51785|C0452162|C0452163|C0494056,Brain white matter_STON1-GTF2A1L,1647); (121,C0041333|C0242172|C0348147|C0348149|C0348905|C0451784|C04517 85|C0452162|C0452163|C0494056,Endometrial tissue_TMEM159,1635); (121,C0041333|C0242172|C0348147|C0348149|C0348905|C0451784| C0451785|C0452162|C0452163|C0494056,Endometrial tissue_GLB1,1550); (121,C0041333|C0242172|C0348147|C0348149|C0348905|C0451784|C045 1785|C0452162|C0452163|C0494056,Monocyte PDHX,1494); (121,C0041333|C0242172|C0348 147|C0348149|C0348905|C0451784|C0451785|C0452162|C0452163|C0494056,Bladder_THEM 6,1490); (121,C0041333|C0242172|C0348147|C0348149|C0348905|C0451784|C0451785|C0452 162|C0452163|C0494056,Temporal_cortex_MYADML2,1475); (121,C0041333|C0242172|C034 8147|C0348149|C0348905|C0451784|C0451785|C0452162|C0452163|C0494056,Brain white matter_CLECe12A,1473); (121,C0041333|C0242172|C0348147|C0348149|C0348905|C0451784|C0451785|C0452162|C0452163|C0494056,Macrophage_MEPE,1424); (121,C0041333|C024217 etrial tissue_PODN,1416); (123,C00429001|C0348510,Whole_blood_AKIRIN2,2911); (123,C00429001|C0348510,Monocyte PER2,2255); (123,C00429001|C0348510,Colon_RAMP2,2035); (123,C0042 900|C0348510,Unknown_CD86,1905); (123,C0042900|C0348510,Monocyte_RILP,1893); (123, C00429001|C0348510,Monocyte_SNX9,1875); (123,C00429001|C0348510,Endometrial tissue_TVP23CP2,1868); (123,C00429001|C0348510,Monocyte_EIF3G,1705); (123,C00042900|C0 348510,Endometrial tissue_SMC2,1699); (123,C00429001|C0348510,Temporal_cortex_TRIM37,1687); (123,C004290 O|C0348510,Colorectum_VPS37B,1656); (123,C000429001|C0348510,Brain white matter_STON1-GTF2A1L,1647); (123,C00429001|C0348510,Endometrial tissue_TMEM159,1635); (123,C00429001|C0348510,Endometrial tissue_GLB1,1550); (123,C00429001|C0348510,Monocyte_PDHX,1494); (123,C00429001|C03485 10,Bladder_THEM6,1490); (123,C00429001|C0348510,Temporal_cortex_MYADML2,1475); (12 3,C000429001|C0348510,Brain white matter_CLEC12A,1473); (123,C00429001|C0348510,Macrophage_MEPE,1424); (123,C00429001|C0348510,Endometrial tissue_PODN,1416); (126,C000852071|C0158915|C0477897|C1455664,Whole_blood_AKIRIN2,2 911); (126,C00085207|C0158915|C0477897|C1455664,Whole_blood_AKIRIN2,2911); (126,C008 5207|C0158915|C0477897|C1455664,Monocyte_PER2,2255); (126,C0085207|C0158915|C0477 897|C1455664,Monocyte_PER2,2255); (126,C00085207|C0158915|C0477897|C1455664,Colon_RAMP2,2035); (126,C00085207|C0158915|C0477897|C1455664,Colon_RAMP2,2035); (126,C00 85207|C0158915|C0477897|C1455664,Unknown_CD86,1905); (126,C0085207|C0158915|C047 7897|C1455664,Unknown_CD86,1905); (126,C00085207|C0158915|C0477897|C1455664,Monoc yte_RILP,1893); (126,C00085207|C0158915|C0477897|C1455664,Monocyte_RILP,1893); (126,C 0085207|C0158915|C0477897|C1455664,Monocyte_SNX9,1875); (126,C0085207|C0158915|C0 477897|C1455664,Monocyte_SNX9,1875); (126,C00085207|C0158915|C0477897|C1455664,End ometrial tissue_TVP23CP2,1868); (126,C00085207|C0158915|C0477897|C1455664,Endometrial tissue_TVP23CP2,1868); (126,C00085207|C0158915|C0477897|C1455664,Monocyte_EIF3G,170 5); (126,C000852071|C0158915|C0477897|C1455664,Monocyte_EIF3G,1705); (126,C0085207|C01 58915|C0477897|C1455664,Endometrial tissue_SMC2,1699); (126,C00085207|C0158915|C0477897|C1455664,Endometrial tissue_SMC2,1699); (126,C00085207|C0158915|C0477897|C1455664,Temporal_cortex_TRIM37 ,1687); (126,C0085207|C0158915|C0477897|C1455664,Temporal_cortex_TRIM37,1687); (132,C 0155709,Whole_blood_AKIRIN2,2911); (132,C0155709,Whole_blood_AKIRIN2,2911); (132,C 0155709,Whole_blood_AKIRIN2,2911); (132,C0155709,Whole_blood_AKIRIN2,2911); (132,C 0155709,Monocyte_PER2,2255); (132,C0155709,Monocyte_PER2,2255); (132,C0155709,Mono cyte_PER2,2255); (132,C0155709,Monocyte_PER2,2255); (132,C0155709,Colon_RAMP2,2035) ; (132,C0155709,Colon_RAMP2,2035); (132,C0155709,Colon_RAMP2,2035); (132,C0155709,C olon_RAMP2,2035); (132,C0155709,Unknown_CD86,1905); (132,C0155709,Unknown_CD86,1 905); (132,C0155709,Unknown_CD86,1905); (132,C0155709,Unknown_CD86,1905); (132,C015 5709,Monocyte_RILP,1893); (132,C0155709,Monocyte_RILP,1893); (132,C0155709,Monocyte_RILP,1893); (132,C0155709,Monocyte_RILP,1893); (133,C0156258|C0178288|C0451641,Whol e_blood_AKIRIN2,2911); (133,C01562581|C01782881|C0451641,Monocyte_PER2,2255); (133,C0 156258|C0178288|C0451641,Colon_RAMP2,2035); (133,C0156258|C0178288|C0451641,Unkn own_CD86,1905); (133,C0156258|C0178288|C0451641,Monocyte_RILP,1893); (133,C01562581|C0178288|C0451641,Monocyte_SNX9,1875); (133,C0156258|C0178288|C0451641,Endometrial tissue_TVP23CP2,1868); (133,C0156258|C0178288|C0451641,Monocyte_EIF3G,1705); (133,C0 1562581|C01782881|C0451641,Endometrial tissue_SMC2,1699); (133,C0156258|C0178288|C0451641,Temporal_cortex_TRIM37,1687); (13 3,C01562581|C01782881|C0451641,Colorectum_VPS37B,1656); (133,C0156258|C0178288|C0451 641,Brain white matter_STON1-GTF2A1L,1647); (133,C0156258|C0178288|C0451641,Endometrial tissue_TMEM159,1635); (133,C0156258|C0178288|C0451641,Endometrial tissue_GLB1,1550); (133,C0156258|C0178288|C0451641,Monocyte_PDHX,1494); (133,C01562 581|C01782881|C0451641,Bladder_THEM6,1490); (133,C0156258|C0178288|C0451641,Temporal_cortex_MYADML2,1475); (133,C0156258|C0178288|C0451641,Brain white matter_CLEC12A,1473); (133,C0156258|C0178288|C0451641,Macrophage_MEPE,1424); (133, C01562581|C01782881|C0451641,Endometrial tissue_PODN,1416); (135,C0178283,Whole_blood_AKIRIN2,1018); (135,C0178283,Monocyte_EIF3G,818); (135,C0178283,Monocyte PER2,817); (135,C0178283,Bladder_TBC1D27,782); (13 5,C0178283,Breast_PARP9,781); (135,C0178283,Whole_blood_AMN1,721); (135,C0178283,Sk in_DPP3,712); (135,C0178283,Whole_blood_CRISP3,700); (135,C0178283,Colorectum_USP2,6 78); (135,C0178283,Skin_GUSBP1,678); (135,C0178283,Whole_blood_ANXA3,673); (135,C017 8283,Whole_blood_CLEC4D,670); (135,C0178283,Monocyte_ANXA11,654); (135,C0178283, Whole_blood_S100A12,654); (135,C0178283,Bladder_MRPL55,650); (135,C0178283,Monocyte PIBF1,650); (135,C0178283,Colorectum_ADCY10,649); (135,C0178283,Adipose tissue_CASS4,649); (135,C0178283,Whole_blood_HNRNPDL,648); (135,C0178283,Colorectum _LINCO1207,639); (137,C0238990, Whole_blood_AKIRIN2,2911); (137,C0238990,Monocyte_P ER2,2255); (137,C0238990,Colon_RAMP2,2035); (137,C0238990,Unknown_CD86,1905); (137, C0238990,Monocyte_RILP,1893); (137,C0238990,Monocyte_SNX9,1875); (137,C0238990,End ometrial tissue_TVP23CP2,1868); (137,C0238990,Monocyte_EIF3G,1705); (137,C0238990,Endometrial tissue_SMC2,1699); (137,C0238990,Temporal_cortex_TRIM37,1687); (137,C0238990,Colorectu m_VPS37B,1656); (137,C0238990,Brain white matter_STON1-GTF2A1L,1647); (137,C0238990,Endometrial tissue_TMEM159,1635); (137,C0238990,Endometrial tissue_GLB1,1550); (137,C0238990,Monocyte_PDHX,1494); (137,C0238990,Bladder_THEM6, 1490); (137,C0238990,Temporal_cortex_MYADML2,1475); (137,C0238990,Brain white matter_CLEC12A,1473); (137,C0238990,Macrophage_MEPE,1424); (137,C0238990,Endometria 1 tissue_PODN,1416); (143,C0276609|C0276610|C0348199|C0400913|C0400918|C0451704|C045 1705|C0494092|C0494093,Whole_blood_AKIRIN2,2911); (143,C0276609|C0276610|C0348199| C0400913|C04009181|C04517041|C0451705|C0494092|C0494093,Monocyte PER2,2255); (143,C 0276609|C0276610|C0348199|C0400913|C0400918|C0451704|C0451705|C0494092|C0494093, Colon_RAMP2,2035); (143,C0276609|C0276610|C0348199|C0400913|C0400918|C0451704|C0 451705|C0494092|C0494093,Unknown_CD86,1905); (143,C0276609|C0276610|C0348199|C04 00913|C0400918|C0451704|C0451705|C0494092|C0494093,Monocyte_RILP,1893); (143,C0276 609|C0276610|C0348199|C0400913|C0400918|C0451704|C0451705|C0494092|C0494093,Mon ocyte_SNX9,1875); (143,C0276609|C0276610|C0348199|C0400913|C0400918|C0451704|C045 1705|C0494092|C0494093,Endometrial tissue_TVP23CP2,1868); (143,C0276609|C0276610|C0348199|C0400913|C0400918|C0451704| C0451705|C0494092|C0494093,Monocyte_EIF3G,1705); (143,C0276609|C0276610|C0348199| C0400913|C0400918|C0451704|C0451705|C0494092|C0494093,Endometrial tissue_SMC2,1699); (143,C0276609|C0276610|C0348199|C0400913|C0400918|C0451704|C045 1705|C0494092|C0494093,Temporal_cortex_TRIM37,1687); (143,C02766091|C02766101|C03481 99|C0400913|C0400918|C0451704|C0451705|C0494092|C0494093,Colorectum_VPS37B,1656) ; (143,C0276609|C0276610|C0348199|C0400913|C0400918|C0451704|C0451705|C0494092|C04 94093,Brain white matter_STON1-GTF2A1L,1647); (143,C0276609|C0276610|C0348199|C0400913|C0400918|C0451704|C04517 05|C0494092|C0494093,Endometrial tissue_TMEM159,1635); (143,C0276609|C0276610|C0348199|C0400913|C0400918|C0451704| C0451705|C0494092|C0494093,Endometrial tissue_GLB1,1550); (143,C0276609|C02766101|C0348199|C0400913|C0400918|C0451704|C045 1705|C0494092|C0494093,Monocyte_PDHX,1494); (143,C0276609|C0276610|C0348199|C0400 913|C0400918|C0451704|C0451705|C0494092|C0494093,Bladder_THEM6,1490); (143,C02766 09|C0276610|C0348199|C0400913|C0400918|C0451704|C0451705|C0494092|C0494093,Temp oral_cortex_MYADML2,1475); (143,C0276609|C02766101|C0348199|C0400913|C0400918|C04 517041|C0451705|C0494092|C0494093,Brain white matter_CLEC12A,1473); (143,C0276609|C02766101|C0348199|C0400913|C0400918|C04517041|C0451705|C0494092|C0494093,Macrophage_MEPE,1424); (143,C0276609|C02766101|C034819 91|C0400913|1|C0400918|1|C04517041|C0451705|1|C0494092| C0494093,Endometrial tissue_PODN,1416); (151,C0348694,Whole_blood_AKIRIN2,2911); (151,C0348694,Monocyte_PER2,2255); (151,C0348694,Colon_RAMP2,2035); (151,C0348694,Unknown_CD86,1905); (151,C0348694,Monocyte_RILP,1893); (151,C0348694,Monocyte_SNX9,1875); (151,C0348694,End ometrial tissue_TVP23CP2,1868); (151,C0348694,Monocyte_EIF3G,1705); (151,C0348694,Endometrial tissue_SMC2,1699); (151,C0348694,Temporal_cortex_TRIM37,1687); (151,C0348694,Colorectu m_VPS37B,1656); (151,C0348694,Brain white matter_STON1-GTF2A1L,1647); (151,C0348694,Endometrial tissue_TMEM159,1635); (151,C0348694,Endometrial tissue_GLB1,1550); (151,C0348694,Monocyte_PDHX,1494); (151,C0348694,Bladder_THEM6, 1490); (151,C0348694,Temporal_cortex_MYADML2,1475); (151,C0348694,Brain white matter_CLEC12A,1473); (151,C0348694,Macrophage_MEPE,1424); (151,C0348694,Endometria 1 tissue_PODN,1416); (153,C0348723|C0451753|C047741 1|C0478155|C0494697|C1527336,Whol e_blood_AKIRIN2,2911); (153,C0348723|C0451753|C0477411|C0478155|C0494697|C1527336, Whole_blood_AKIRIN2,2911); (153,C0348723|C0451753|C0477411|C0478155|C0494697|C152 7336,Monocyte_PER2,2255); (153,C0348723|C0451753|C0477411|C0478155|C0494697|C1527 336,Monocyte_PER2,2255); (153,C0348723|C0451753|C0477411|C0478155|C0494697|C15273 36,Colon_RAMP2,2035); (153,C0348723|C0451753|C0477411|C0478155|C0494697|C1527336, Colon_RAMP2,2035); (153,C0348723|C0451753|C0477411|C0478155|C0494697|C1527336,Un known_CD86,1905); (153,C0348723|C0451753|C0477411|C0478155|C0494697|C1527336,Unk nown_CD86,1905); (153,C0348723|C0451753|C0477411|C0478155|C0494697|C1527336,Mono cyte_RILP,1893); (153,C0348723|C0451753|C0477411|C0478155|C0494697|C1527336,Monocy te_RILP,1893); (153,C0348723|C0451753|C0477411|C0478155|C0494697|C1527336,Monocyte _SNX9,1875); (153,C0348723|C0451753|C047741|C0478155|C0494697|C1527336,Monocyte_SNX9,1875); (153,C0348723|C0451753|C047741|C0478155|C0494697|C1527336,Endometrial tissue_TVP23CP2,1868); (153,C0348723|C0451753|C0477411|C0478155|C0494697|C1527336, Endometrial tissue_TVP23CP2,1868); (153,C0348723|C0451753|C0477411|C0478155|C0494697|C1527336, Monocyte_EIF3G,1705); (153,C0348723|C0451753|C0477411|C0478155|C0494697|C1527336, Monocyte_EIF3G,1705); (153,C0348723|C0451753|C0477411|C0478155|C0494697|C1527336, Endometrial tissue_SMC2,1699); (153,C0348723|C0451753|C0477411|C0478155|C0494697|C1527336,Endo metrial tissue_SMC2,1699); (153,C0348723|C0451753|C0477411|C0478155|C0494697|C1527336,Temp oral_cortex_TRIM37,1687); (153,C0348723|C0451753|C0477411|C0478155|C0494697|C15273 36,Temporal_cortex_TRIM37,1687); (156,C03493491|C0579986,Whole_blood_AKIRIN2,2911); (156,C0349349|C0579986,Whole_blood_AKIRIN2,2911); (156,C0349349|C0579986,Whole_bl ood_AKIRIN2,2911); (156,C03493491|C0579986,Monocyte_PER2,2255); (156,C03493491|C0579 986,Monocyte_PER2,2255); (156,C03493491|C0579986,Monocyte_PER2,2255); (156,C03493491|C0579986,Colon_RAMP2,2035); (156,C03493491|C0579986,Colon_RAMP2,2035); (156,C03493 49|C0579986,Colon_RAMP2,2035); (156,C0349349|C0579986,Unknown_CD86,1905); (156,C0 349349|C0579986,Unknown_CD86,1905); (156,C0349349|C0579986,Unknown_CD86,1905); (1 56,C0349349|C0579986,Monocyte_RILP,1893); (156,C0349349|C0579986,Monocyte_RILP,189 3); (156,C0349349|C0579986,Monocyte_RILP,1893); (156,C0349349|C0579986,Monocyte_SN X9,1875); (156,C0349349|C0579986,Monocyte_SNX9,1875); (156,C0349349|C0579986,Monoc yte_SNX9,1875); (156,C03493491|C0579986,Endometrial tissue_TVP23CP2,1868); (156,C03493491|C0579986,Endometrial tissue_TVP23CP2,1868); (157,C04009141|C0524910,Whole_blood_AKIRIN2,2911); (157,C0400 9141|C0524910,Monocyte_PER2,2255); (157,C04009141|C0524910,Colon_RAMP2,2035); (157,C 0400914|C0524910,Unknown_CD86,1905); (157,C0400914|C0524910,Monocyte_RILP,1893); (157,C0400914|C0524910,Monocyte_SNX9,1875); (157,C0400914|C0524910,Endometrial tissue_TVP23CP2,1868); (157,C0400914|C0524910,Monocyte_EIF3G,1705); (157,C0400914|C0 524910,Endometrial tissue_SMC2,1699); (157,C04009141|C0524910,Temporal_cortex_TRIM37,1687); (157,C040091 41|C0524910,Colorectum_VPS37B,1656); (157,C0400914|C0524910,Brain white matter_STON1-GTF2A1L,1647); (157,C04009141|C0524910,Endometrial tissue_TMEM159,1635); (157,C04009141|C0524910,Endometrial tissue_GLB1,1550); (157,C04009141|C0524910,Monocyte_PDHX,1494); (157,C0400914|C05249 10,Bladder_THEM6,1490); (157,C04009141|C0524910,Temporal_cortex_MYADML2,1475); (15 7,C04009141|C0524910,Brain white matter_CLEC12A,1473); (157,C0400914|C0524910,Macrophage_MEPE,1424); (157,C0400914| C0524910,Endometrial tissue_PODN,1416); (158,C0404572,Whole_blood_AKIRIN2,2911); (158,C0404572,Monocyte_PER2,2255); (158,C0404572,Colon_RAMP2,2035); (158,C0404572,Unknown_CD86,1905); (158,C0404572,Monocyte_RILP,1893); (158,C0404572,Monocyte_SNX9,1875); (158,C0404572,End ometrial tissue_TVP23CP2,1868); (158,C0404572,Monocyte_EIF3G,1705); (158,C0404572,Endometrial tissue_SMC2,1699); (158,C0404572,Temporal_cortex_TRIM37,1687); (158,C0404572,Colorectu m_VPS37B,1656); (158,C0404572,Brain white matter_STON1-GTF2A1L,1647); (158,C0404572,Endometrial tissue_TMEM159,1635); (158,C0404572,Endometrial tissue_GLB1,1550); (158,C0404572,Monocyte_PDHX,1494); (158,C0404572,Bladder_THEM6, 1490); (158,C0404572,Temporal_cortex_MYADML2,1475); (158,C0404572,Brain white matter_CLEC12A,1473); (158,C0404572,Macrophage_MEPE,1424); (158,C0404572,Endometria 1 tissue_PODN,1416); (159,C0433856|C0452047,Whole_blood_AKIRIN2,2911); (159,C04338561|C0452047,Monocyte_PER2,2255); (159,C0433856|C0452047,Colon_RAMP2,2035); (159,C0433 856|C0452047,Unknown_CD86,1905); (159,C0433856|C0452047,Monocyte_RILP,1893); (159, C0433856|C0452047,Monocyte_SNX9,1875); (159,C0433856|C0452047,Endometrial tissue_TVP23CP2,1868); (159,C0433856|C0452047,Monocyte_EIF3G,1705); (159,C0433856|C0 452047,Endometrial tissue_SMC2,1699); (159,C0433856|C0452047,Temporal_cortex_TRIM37,1687); (159,C043385 6|C0452047,Colorectum_VPS37B,1656); (159,C0433856|C0452047,Brain white matter_STON1-GTF2A1L,1647); (159,C0433856|C0452047,Endometrial tissue_TMEM159,1635); (159,C0433856|C0452047,Endometrial tissue_GLB1,1550); (159,C0433856|C0452047,Monocyte_PDHX,1494); (159,C0433856|C04520 47,Bladder_THEM6,1490); (159,C0433856|C0452047,Temporal_cortex_MYADML2,1475); (15 9,C0433856|C0452047,Brain white matter_CLEC12A,1473); (159,C0433856|C0452047,Macrophage_MEPE,1424); (159,C04338561|C0452047,Endometrial tissue_PODN,1416); (160,C04517071|C0728889,Whole_blood_AKIRIN2,2911); (160,C04517071 C0728889,Monocyte_PER2,2255); (160,C04517071|C0728889,Colon_RAMP2,2035); (160,C0451 707|C0728889,Unknown_CD86,1905); (160,C0451707|C0728889,Monocyte_RILP,1893); (160, C0451707|C0728889,Monocyte_SNX9,1875); (160,C0451707|C0728889,Endometrial tissue_TVP23CP2,1868); (160,C0451707|C0728889,Monocyte_EIF3G,1705); (160,C0451707|C0 728889,Endometrial tissue_SMC2,1699); (160,C0451707|C0728889,Temporal_cortex_TRIM37,1687); (160,C045170 71|C0728889,Colorectum_VPS37B,1656); (160,C0451707|C0728889,Brain white matter_STON1-GTF2A1L,1647); (160,C0451707|C0728889,Endometrial tissue_TMEM159,1635); (160,C0451707|C0728889,Endometrial tissue_GLB1,1550); (160,C0451707|C0728889,Monocyte PDHX,1494); (160,C0451707|C07288 89,Bladder_THEM6,1490); (160,C0451707|C0728889,Temporal_cortex_MYADML2,1475); (16 0,C04517071|C0728889,Brain white matter_CLEC12A,1473); (160,C0451707|C0728889,Macrophage_MEPE,1424); (160,C0451707| C0728889,Endometrial tissue_PODN,1416); (161,C0451708|C0451710|C0451711|C0451712|C0494794,Whole_blood_AKIRIN2,2911); (161,C0451708|C0451710|C0451711|C0451712|C0494794,Monocyte_PER2,22 55); (161,C0451708|C0451710|C0451711|C0451712|C0494794,Colon_RAMP2,2035); (161,C045 1708|C0451710|C0451711|C0451712|C0494794,Unknown_CD86,1905); (161,C0451708|C0451 710|C045171 1|C0451712|C0494794,Monocyte_RILP,1893); (161,C0451708|C0451710|C045171 1|C0451712|C0494794,Monocyte_SNX9,1875); (161,C0451708|C0451710|C0451711|C0451712| C0494794,Endometrial tissue_TVP23CP2,1868); (161,C0451708|C0451710|C0451711|C0451712|C0494794,Monocyte_EIF3G,1705); (161,C0451708|C0451710|C0451711|C0451712|C0494794,Endometrial tissue_SMC2,1699); (161,C0451708|C0451710|C0451711|C0451712|C0494794,Temporal_corte x_TRIM37,1687); (161,C0451708|C0451710|C0451711|C0451712|C0494794,Colorectum_VPS3 7B,1656); (161,C0451708|C04517101|C0451711|C0451712|C0494794,Brain white matter_STON1-GTF2A1L,1647); (161,C0451708|C0451710|C045171|C0451712|C0494794,Endometrial tissue_TMEM159,1635); (161,C0451708|C0451710|C0451711|C0451712|C0494794,Endometria 1 tissue_GLB1,1550); (161,C0451708|C0451710|C0451711|C0451712|C0494794,Monocyte_PDH X,1494); (161,C0451708|C0451710|C0451711|C0451712|C0494794,Bladder_THEM6,1490); (16 1,C0451708|C0451710|C0451711|C0451712|C0494794,Temporal_cortex_MYADML2,1475); (1 61,C0451708|C04517101|C0451711|C0451712|C0494794,Brain white matter_CLEC12A,1473); (161,C0451708|C04517101|C0451711|C0451712|C0494794,Macrophag e_MEPE,1424); (161,C0451708|C04517101|C0451711|C0451712|C0494794,Endometrial tissue_PODN,1416); (165,C0477491,Whole_blood_AKIRIN2,2911); (165,C0477491,Whole_blo od_AKIRIN2,2911); (165,C0477491,Monocyte PER2,2255); (165,C0477491,Monocyte_PER2,2 255); (165,C0477491,Colon_RAMP2,2035); (165,C0477491,Colon_RAMP2,2035); (165,C04774 91,Unknown_CD86,1905); (165,C0477491,Unknown_CD86,1905); (165,C0477491,Monocyte_R ILP,1893); (165,C0477491,Monocyte_RILP,1893); (165,C0477491,Monocyte_SNX9,1875); (165, C0477491,Monocyte_SNX9,1875); (165,C0477491,Endometrial tissue_TVP23CP2,1868); (165,C0477491,Endometrial tissue_TVP23CP2,1868); (165,C0477491,Monocyte_EIF3G,1705); (165,C0477491,Monocyte_EI F3G,1705); (165,C0477491,Endometrial tissue_SMC2,1699); (165,C0477491,Endometrial tissue_SMC2,1699); (165,C0477491,Temporal_cortex_TRIM37,1687); (165,C0477491,Temporal_cortex_TRIM37,1687); (166,C0494026,Whole_blood_AKIRIN2,2911); (166,C0494026,Monoc yte_PER2,2255); (166,C0494026,Colon_RAMP2,2035); (166,C0494026,Unknown_CD86,1905); (166,C0494026,Monocyte_RILP,1893); (166,C0494026,Monocyte_SNX9,1875); (166,C0494026,Endometrial tissue_TVP23CP2,1868); (166,C0494026,Monocyte_EIF3G,1705); (166,C0494026,Endometrial tissue_SMC2,1699); (166,C0494026,Temporal_cortex_TRIM37,1687); (166,C0494026,Colorectu m_VPS37B,1656); (166,C0494026,Brain white matter_STON1-GTF2A1L,1647); (166,C0494026,Endometrial tissue_TMEM159,1635); (166,C0494026,Endometrial tissue_GLB1,1550); (166,C0494026,Monocyte_PDHX,1494); (166,C0494026,Bladder_THEM6, 1490); (166,C0494026,Temporal_cortex_MYADML2,1475); (166,C0494026,Brain white matter_CLEC12A,1473); (166,C0494026,Macrophage_MEPE,1424); (166,C0494026,Endometria 1 tissue_PODN,1416); (169,C04946501|C0494660,Whole_blood_AKIRIN2,2911); (169,C04946501|C0494660,Monocyte_PER2,2255); (169,C04946501|C0494660,Colon_RAMP2,2035); (169,C0494 650|C0494660,Unknown_CD86,1905); (169,C0494650|C0494660,Monocyte_RILP,1893); (169, C0494650|C0494660,Monocyte_SNX9,1875); (169,C0494650|C0494660,Endometrial tissue_TVP23CP2,1868); (169,C0494650|C0494660,Monocyte_EIF3G,1705); (169,C0494650|C0 494660,Endometrial tissue_SMC2,1699); (169,C04946501|C0494660,Temporal_cortex_TRIM37,1687); (169,C049465 0|C0494660,Colorectum_VPS37B,1656); (169,C04946501|C0494660,Brain white matter_STON1-GTF2A1L,1647); (169,C04946501|C0494660,Endometrial tissue_TMEM159,1635); (169,C04946501|C0494660,Endometrial tissue_GLB1,1550); (169,C04946501|C0494660,Monocyte PDHX,1494); (169,C04946501|C04946 60,Bladder_THEM6,1490); (169,C04946501|C0494660,Temporal_cortex_MYADML2,1475); (16 9,C04946501|C0494660,Brain white matter_CLEC12A,1473); (169,C0494650|C0494660,Macrophage_MEPE,1424); (169,C04946501|C0494660,Endometrial tissue_PODN,1416); (171,C04946971|C04948871|C0495047|C3495801,Whole_blood_AKIRIN2,2 911); (171,C0494697|C0494887|C0495047|C3495801,Whole_blood_AKIRIN2,2911); (171,C049 4697|C0494887|C0495047|C3495801,Monocyte_PER2,2255); (171,C0494697|C0494887|C0495 0471|C3495801,Monocyte PER2,2255); (171,C0494697|C0494887|C0495047|C3495801,Colon_RAMP2,2035); (171,C0494697|C0494887|C0495047|C3495801,Colon_RAMP2,2035); (171,C04 94697|C0494887|C0495047|C3495801,Unknown_CD86,1905); (171,C0494697|C0494887|C049 5047|C3495801,Unknown_CD86,1905); (171,C0494697|C0494887|C0495047|C3495801,Monoc yte_RILP,1893); (171,C0494697|C0494887|C0495047|C3495801,Monocyte_RILP,1893); (171,C 0494697|C0494887|C0495047|C3495801,Monocyte_SNX9,1875); (171,C0494697|C0494887|C0 495047|C3495801,Monocyte_SNX9,1875); (171,C0494697|C0494887|C0495047|C3495801,End ometrial tissue_TVP23CP2,1868); (171,C0494697|C0494887|C0495047|C3495801,Endometrial tissue_TVP23CP2,1868); (171,C0494697|C0494887|C0495047|C3495801,Monocyte_EIF3G,170 5); (171,C04946971|C04948871|C0495047|C3495801,Monocyte_EIF3G,1705); (171,C0494697|C04 94887|C0495047|C3495801,Endometrial tissue_SMC2,1699); (171,C0494697|C0494887|C0495047|C3495801,Endometrial tissue_SMC2,1699); (171,C0494697|C0494887|C0495047|C3495801,Temporal_cortex_TRIM37 ,1687); (171,C0494697|C0494887|C0495047|C3495801,Temporal_cortex_TRIM37,1687); (172,C 0494783,Whole_blood_AKIRIN2,2911); (172,C0494783,Monocyte_PER2,2255); (172,C049478 3,Colon_RAMP2,2035); (172,C0494783,Unknown_CD86,1905); (172,C0494783,Monocyte_RIL P,1893); (172,C0494783,Monocyte_SNX9,1875); (172,C0494783,Endometrial tissue_TVP23CP2,1868); (172,C0494783,Monocyte_EIF3G,1705); (172,C0494783,Endometrial tissue_SMC2,1699); (172,C0494783,Temporal_cortex_TRIM37,1687); (172,C0494783,Colorectu m_VPS37B,1656); (172,C0494783,Brain white matter_STON1-GTF2A1L,1647); (172,C0494783,Endometrial tissue_TMEM159,1635); (172,C0494783,Endometrial tissue_GLB1,1550); (172,C0494783,Monocyte_PDHX,1494); (172,C0494783,Bladder_THEM6, 1490); (172,C0494783,Temporal_cortex_MYADML2,1475); (172,C0494783,Brain white matter_CLEC12A,1473); (172,C0494783,Macrophage_MEPE,1424); (172,C0494783,Endometria 1 tissue_PODN,1416); (173,C0494831,Whole_blood_AKIRIN2,2911); (173,C0494831,Whole_blo od_AKIRIN2,2911); (173,C0494831,Whole_blood_AKIRIN2,2911); (173,C0494831,Monocyte_PER2,2255); (173,C0494831,Monocyte PER2,2255); (173,C0494831,Monocyte_PER2,2255); (1 73,C0494831,Colon_RAMP2,2035); (173,C0494831,Colon_RAMP2,2035); (173,C0494831,Colo n_RAMP2,2035); (173,C0494831,Unknown_CD86,1905); (173,C0494831,Unknown_CD86,1905); (173,C0494831,Unknown_CD86,1905); (173,C0494831,Monocyte_RILP,1893); (173,C049483 1,Monocyte_RILP,1893); (173,C0494831,Monocyte_RILP,1893); (173,C0494831,Monocyte_SN X9,1875); (173,C0494831,Monocyte_SNX9,1875); (173,C0494831,Monocyte_SNX9,1875); (173,C0494831,Endometrial tissue_TVP23CP2,1868); (173,C0494831,Endometrial tissue_TVP23CP2,1868); (175,C0496594,Whole_blood_AKIRIN2,2911); (175,C0496594,Monoc yte_PER2,2255); (175,C0496594,Colon_RAMP2,2035); (175,C0496594,Unknown_CD86,1905); (175,C0496594,Monocyte_RILP,1893); (175,C0496594,Monocyte_SNX9,1875); (175,C0496594,Endometrial tissue_TVP23CP2,1868); (175,C0496594,Monocyte_EIF3G,1705); (175,C0496594,Endometrial tissue_SMC2,1699); (175,C0496594,Temporal_cortex_TRIM37,1687); (175,C0496594,Colorectu m_VPS37B,1656); (175,C0496594,Brain white matter_STON1-GTF2A1L,1647); (175,C0496594,Endometrial tissue_TMEM159,1635); (175,C0496594,Endometrial tissue_GLB1,1550); (175,C0496594,Monocyte_PDHX,1494); (175,C0496594,Bladder_THEM6, 1490); (175,C0496594,Temporal_cortex_MYADML2,1475); (175,C0496594,Brain white matter_CLEC12A,1473); (175,C0496594,Macrophage_MEPE,1424); (175,C0496594,Endometria 1 tissue_PODN,1416); (176,C0496856,Whole_blood_AKIRIN2,2911); (176,C0496856,Monocyte_PER2,2255); (176,C0496856,Colon_RAMP2,2035); (176,C0496856,Unknown_CD86,1905); (176,C0496856,Monocyte_RILP,1893); (176,C0496856,Monocyte_SNX9,1875); (176,C0496856,End ometrial tissue_TVP23CP2,1868); (176,C0496856,Monocyte_EIF3G,1705); (176,C0496856,Endometrial tissue_SMC2,1699); (176,C0496856,Temporal_cortex_TRIM37,1687); (176,C0496856,Colorectu m_VPS37B,1656); (176,C0496856,Brain white matter_STON1-GTF2A1L,1647); (176,C0496856,Endometrial tissue_TMEM159,1635); (176,C0496856,Endometrial tissue_GLB1,1550); (176,C0496856,Monocyte_PDHX,1494); (176,C0496856,Bladder_THEM6, 1490); (176,C0496856,Temporal_cortex_MYADML2,1475); (176,C0496856,Brain white matter_CLEC12A,1473); (176,C0496856,Macrophage_MEPE,1424); (176,C0496856,Endometria 1 tissue_PODN,1416); (179,C1265999,Whole_blood_AKIRIN2,2911); (179,C1265999,Whole_blo od_AKIRIN2,2911); (179,C1265999,Monocyte PER2,2255); (179,C1265999,Monocyte_PER2,2 255); (179,C1265999,Colon_RAMP2,2035); (179,C1265999,Colon_RAMP2,2035); (179,C12659 99,Unknown_CD86,1905); (179,C1265999,Unknown_CD86,1905); (179,C1265999,Monocyte_R ILP,1893); (179,C1265999,Monocyte_RILP,1893); (179,C1265999,Monocyte_SNX9,1875); (179, C1265999,Monocyte_SNX9,1875); (179,C1265999,Endometrial tissue_TVP23CP2,1868); (179,C1265999,Endometrial tissue_TVP23CP2,1868); (179,C1265999,Monocyte_EIF3G,1705); (179,C1265999,Monocyte_EI F3G,1705); (179,C1265999,Endometrial tissue_SMC2,1699); (179,C1265999,Endometrial tissue_SMC2,1699); (179,C1265999,Temporal_cortex_TRIM37,1687); (179,C1265999,Temporal_cortex_TRIM37,1687); (186,barrett oesophagus,Whole_blood_AKIRIN2,2911); (186,barrett oesophagus,Monocyte_PER2,2255); (186,barrett oesophagus,Colon_RAMP2,2035); (186,barrett oesophagus,Unknown_CD86,1905); (186,barrett oesophagus,Monocyte_RILP,1893); (186,barrett oesophagus,Monocyte_SNX9,1875); (186,barrett oesophagus,Endometrial tissue_TVP23CP2,1868); (186,barrett oesophagus,Monocyte_EIF3G,1705); (186,barrett oesophagus,Endometrial tissue_SMC2,1699); (186,barrett oesophagus,Temporal_cortex_TRIM37,1687); (186,barrett oesophagus,Colorectum_VPS37B,1656); (186,barrett oesophagus,Brain white matter_STON1-GTF2A1L,1647); (186,barrett oesophagus,Endometrial tissue_TMEM159,1635); (186,barrett oesophagus,Endometrial tissue_GLB1,1550); (186,barrett oesophagus,Monocyte_PDHX,1494); (186,barrett oesophagus,Bladder_THEM6,1490); (186,barrett oesophagus,Temporal_cortex_MYADML2,1475); (186,barrett oesophagus,Brain white matter_CLEC12A,1473); (186,barrett oesophagus,Macrophage_MEPE,1424); (186,barrett oesophagus,Endometrial tissue_PODN,1416); (191,dandruff,Whole_blood_AKIRIN2,2911); (191,dandruff,Monocyte_PE R2,2255); (191,dandruff,Colon_RAMP2,2035); (191,dandruff,Unknown_CD86,1905); (191,dandr uff,Monocyte_RILP,1893); (191,dandruff,Monocyte_SNX9,1875); (191,dandruff,Endometrial tissue_TVP23CP2,1868); (191,dandruff,Monocyte_EIF3G,1705); (191,dandruff,Endometrial tissue_SMC2,1699); (191,dandruff,Temporal_cortex_TRIM37,1687); (191,dandruff,Colorectum_VPS37B,1656); (191,dandruff,Brain white matter_STON1-GTF2A1L,1647); (191,dandruff,Endometrial tissue_TMEM159,1635); (191,dandruff,Endometrial tissue_GLB1,1550); (191,dandruff,Monocyte_PDHX,1494); (191,dandruff,Bladder_THEM6,149 0); (191,dandruff,Temporal_cortex_MYADML2,1475); (191,dandruff,Brain white matter_CLEC12A,1473); (191,dandruff,Macrophage_MEPE,1424); (191,dandruff,Endometrial tissue_PODN,1416); (200,juvenile idiopathic arthritis,Whole_blood_AKIRIN2,2911); (200,juvenile idiopathic arthritis,Monocyte_PER2,2255); (200,juvenile idiopathic arthritis,Colon_RAMP2,2035); (200,juvenile idiopathic arthritis,Unknown_CD86,1905); (200,juvenile idiopathic arthritis,Monocyte_RILP,1893); (200,juvenile idiopathic arthritis,Monocyte_SNX9,1875); (200,juvenile idiopathic arthritis,Endometrial tissue_TVP23CP2,1868); (200,juvenile idiopathic arthritis,Monocyte_EIF3G,1705); (200,juvenile idiopathic arthritis,Endometrial tissue_SMC2,1699); (200,juvenile idiopathic arthritis,Temporal_cortex_TRIM37,1687); (200,juvenile idiopathic arthritis,Colorectum_VPS37B,1656); (200,juvenile idiopathic arthritis,Brain white matter_STON1-GTF2A1L,1647); (200,juvenile idiopathic arthritis,Endometrial tissue_TMEM159,1635); (200,juvenile idiopathic arthritis,Endometrial tissue_GLB1,1550); (200,juvenile idiopathic arthritis,Monocyte_PDHX,1494); (200,juvenile idiopathic arthritis,Bladder_THEM6,1490); (200,juvenile idiopathic arthritis,Temporal_cortex_MYADML2,1475); (200,juvenile idiopathic arthritis,Brain white matter_CLEC12A,1473); (200,juvenile idiopathic arthritis,Macrophage_MEPE,1424); (200,juvenile idiopathic arthritis,Endometrial tissue_PODN,1416); (209,prostate cancer,Whole_blood_AKIRIN2,2911); (209,prostate cancer,Monocyte_PER2,2255); (209,prostate cancer,Colon_RAMP2,2035); (209,prostate cancer,Unknown_CD86,1905); (209,prostate cancer,Monocyte_RILP,1893); (209,prostate cancer,Monocyte_SNX9,1875); (209,prostate cancer,Endometrial tissue_TVP23CP2,1868); (209,prostate cancer,Monocyte_EIF3G,1705); (209,prostate cancer,Endometrial tissue_SMC2,1699); (209,prostate cancer,Temporal_cortex_TRIM37,1687); (209,prostate cancer,Colorectum_VPS37B,1656); (209,prostate cancer,Brain white matter_STON1-GTF2A1L,1647); (209,prostate cancer,Endometrial tissue_TMEM159,1635); (209,prostate cancer,Endometrial tissue_GLB1,1550); (209,prostate cancer,Monocyte_PDHX,1494); (209,prostate cancer,Bladder_THEM6,1490); (209,prostate cancer,Temporal_cortex_MYADML2,1475); (209,prostate cancer,Brain white matter_CLEC12A,1473); (209,prostate cancer,Macrophage_MEPE,1424); (209,prostate cancer,Endometrial tissue_PODN,1416); (210,reflux esophagitis,Whole_blood_AKIRIN2,2911); (210,reflux esophagitis,Monocyte_PER2,2255); (210,reflux esophagitis,Colon_RAMP2,2035); (210,reflux esophagitis,Unknown_CD86,1905); (210,reflux esophagitis,Monocyte_RILP,1893); (210,reflux esophagitis,Monocyte_SNX9,1875); (210,reflux esophagitis,Endometrial tissue_TVP23CP2,1868); (210,reflux esophagitis,Monocyte_EIF3G,1705); (210,reflux esophagitis,Endometrial tissue_SMC2,1699); (210,reflux esophagitis,Temporal_cortex_TRIM37,1687); (210,reflux esophagitis,Colorectum_VPS37B,1656); (210,reflux esophagitis,Brain white matter_STON1-GTF2A1L,1647); (210,reflux esophagitis,Endometrial tissue_TMEM159,1635); (210,reflux esophagitis,Endometrial tissue_GLB1,1550); (210,reflux esophagitis,Monocyte_PDHX,1494); (210,reflux esophagitis,Bladder_THEM6,1490); (210,reflux esophagitis,Temporal_cortex_MYADML2,1475); (210,reflux esophagitis,Brain white matter_CLEC12A,1473); (210,reflux esophagitis,Macrophage_MEPE,1424); (210,reflux esophagitis,Endometrial tissue_PODN,1416); (212,sjogren syndrome,Whole_blood_AKIRIN2,2911); (212,sjogren syndrome,Monocyte_PER2,2255); (212,sjogren syndrome,Colon_RAMP2,2035); (212,sjogren syndrome,Unknown_CD86,1905); (212,sjogren syndrome,Monocyte_RILP,1893); (212,sjogren syndrome,Monocyte_SNX9,1875); (212,sjogren syndrome,Endometrial tissue_TVP23CP2,1868); (212,sjogren syndrome,Monocyte_EIF3G,1705); (212,sjogren syndrome,Endometrial tissue_SMC2,1699); (212,sjogren syndrome,Temporal_cortex_TRIM37,1687); (212,sjogren syndrome,Colorectum_VPS37B,1656); (212,sjogren syndrome,Brain white matter_STON1-GTF2A1L,1647); (212,sjogren syndrome,Endometrial tissue_TMEM159,1635); (212,sjogren syndrome,Endometrial tissue_GLB1,1550); (212,sjogren syndrome,Monocyte_PDHX,1494); (212,sjogren syndrome,Bladder_THEM6,1490); (212,sjogren syndrome,Temporal_cortex_MYADML2,1475); (212,sjogren syndrome,Brain white matter_CLEC12A,1473); (212,sjogren syndrome,Macrophage_MEPE,1424); (212,sjogren syndrome,Endometrial tissue PODN,1416); (213,stevens-johnson syndrome,Whole_blood_AKIRIN2,2911); (213,stevens-johnson syndrome,Monocyte_PER2,2255); (213,stevens-johnson syndrome,Colon_RAMP2,2035); (213,stevens-johnson syndrome,Unknown_CD86,1905); (213,stevens-johnson syndrome,Monocyte_RILP,1893); (213,stevens-johnson syndrome,Monocyte_SNX9,1875); (213,stevens-johnson syndrome,Endometrial tissue_TVP23CP2,1868); (213,stevens-johnson syndrome,Monocyte_EIF3G,1705); (213,stevens-johnson syndrome,Endometrial tissue_SMC2,1699); (213,stevens-johnson syndrome,Temporal_cortex_TRIM37,1687); (213,stevens-johnson syndrome,Colorectum_VPS37B,1656); (213,stevens-johnson syndrome,Brain white matter_STON1-GTF2A1L,1647); (213,stevens-johnson syndrome,Endometrial tissue_TMEM159,1635); (213,stevens-johnson syndrome,Endometrial tissue_GLB1,1550); (213,stevens-johnson syndrome,Monocyte_PDHX,1494); (213,stevens-johnson syndrome,Bladder_THEM6,1490); (213,stevens-johnson syndrome,Temporal_cortex_MYADML2,1475); (213,stevens-johnson syndrome,Brain white matter_CLEC12A,1473); (213,stevens-johnson syndrome,Macrophage_MEPE,1424); (213,stevens-johnson syndrome,Endometrial tissue_PODN,1416)

### REFERENCE SIGNS LIST

10: search device
100: control unit
1042: search program

1. A method of searching for novel diagnostic biomarkers and/or therapeutic targets, comprising
performing a topic analysis using a topic model on an omics database derived from two or more biofeatures linked to information indicating a disease name to estimate missing biofeatures for a particular disease, wherein
the estimated missing biofeatures suggest the novel diagnostic biomarkers and/or therapeutic targets.

2. The method according to claim 1, wherein the two or more biofeatures are selected from single nucleotide polymorphisms, microbiota, drug targets, transcriptome, proteome and disease biomarkers.

3. The method according to claim 1, wherein the topic model is based on Latent Dirichlet Allocation model.

4. The method according to claim 1, further comprising verifying the reliability of the extracted missing biofeatures.

5. A device of searching for novel diagnostic biomarkers and/or therapeutic targets provided with a control unit, wherein
the control unit performs a topic analysis using a topic model on an omics database derived from two or more biofeatures linked to information indicating a disease name to estimate missing biofeatures for a particular disease, wherein
the estimated missing biofeatures suggest the novel diagnostic biomarkers and/or therapeutic targets.

6. A program of searching for novel diagnostic biomarkers and/or therapeutic targets that, when executed by a computer, causes the computer to execute a step of performing a topic analysis using a topic model on an omics database derived from two or more biofeatures linked to information indicating a disease name to estimate missing biofeatures for a particular disease, wherein
the estimated missing biofeatures suggest the novel diagnostic biomarkers and/or therapeutic targets.
